(19) United States
(12) Patent Application Publication
(10) Pub. No.: US 2024/0150753 A1
(43) Pub. Date: May 9, 2024

(54) METHODS OF ISOTHERMAL COMPLEMENTARY DNA AND LIBRARY PREPARATION

(71) Applicant: Illumina, Inc., San Diego, CA (US)

(72) Inventors: Allison Yunghans, San Diego, CA (US); Angelica Marie Barr Schalembier, San Diego, CA (US); Kayla Busby, San Diego, CA (US); Stephen M. Gross, San Diego, CA (US); Robert Scott Kuersten, Madison, WI (US); Frederick W. Hyde, Monona, WI (US)

(73) Assignee: Illumina, Inc., San Diego, CA (US)

(21) Appl. No.: 18/475,885

(22) Filed: Sep. 27, 2023

Related U.S. Application Data
(63) Continuation of application No. PCT/US2022/022288, filed on Mar. 29, 2022.
(60) Provisional application No. 63/234,114, filed on Aug. 17, 2021.
(60) Provisional application No. 63/167,909, filed on Mar. 30, 2021.

(51) Int. Cl. C12N 15/10; C12N 9/12; C12N 9/22

(52) U.S. Cl. CPC C12N 15/1096; C12N 9/1252; C12N 9/1276; C12N 9/22

(57) ABSTRACT

Described herein are compositions and methods for preparing double-stranded complementary DNA (cDNA) from RNA. In some embodiments, these methods allow isothermal preparation of cDNA. In some embodiments, these methods allow mesophilic or thermostable preparation of cDNA. Also described herein are compositions and methods for preparing cDNA and a library of double-stranded cDNA fragments in a single reaction vessel.

### CROSS-REFERENCE TO RELATED APPLICATIONS

This application is a bypass continuation of PCT/US2022/022288, filed Mar. 29, 2022, which claims the benefit of priority of U.S. Provisional Application No. 63/167,909, filed Mar. 30, 2021, and Application No. 63/234,114, filed Aug. 17, 2021; each of which is incorporated by reference herein in its entirety for any purpose.

### SEQUENCE LISTING

The present application is filed with a Sequence Listing in electronic format. The Sequence Listing is provided as a file entitled “2024-01-17-01243-0026-00PCT” created on Jan. 17, 2024, which is 28,727 bytes in size. The information in the electronic format of the sequence listing is incorporated herein by reference in its entirety.

### DESCRIPTION

### Field

This disclosure relates to improved compositions and methods for double-stranded complementary DNA (ds-cDNA) preparation. These compositions may allow isothermal methods for preparing cDNA from RNA. This disclosure also relates to compositions and methods for preparing ds-cDNA and a library of ds-cDNA fragments.

### Background

RNA is an important biological molecule as its study facilitates understanding of functional biological processes within a cell (i.e., the study of the transcriptome or transcriptomics) and understanding of regulatory elements (such as long non-coding RNAs (lncRNAs) or microRNAs (miRNAs)). Analysis of RNA can also be useful for detection of infectious agents (such as RNA viruses). A prerequisite for study of RNA is often conversion of RNA into a DNA copy, as DNA has properties that enhance its chemical stability and make it amenable to manipulation using common molecular biology tools and reagents. Moreover, for many types of sequencing library preparations, double-stranded DNA is required for ligases used for adapter ligation or transposomes used for adapter addition through tagmentation. Thus, single-stranded RNA must often undergo a conversion into double-stranded complementary DNA (ds-cDNA) prior to library preparation, which adds significantly to turnaround time and hands-on time in RNA workflows.

Further, conversion of RNA into ds-cDNA requires coordinated temperature regulation on a programmable thermal cycler, as shown in FIG. 1. In conventional methods, conversion of RNA into DNA is done through a process of first strand cDNA synthesis by reverse transcription, where the RNA molecule is directly copied by reverse transcriptase. A second strand cDNA is formed by direct replacement of the originating RNA molecules. In most embodiments, this is accomplished through procedures similar to those developed by Gubler and Hoffman Gene 25: 263-269 (1983). Many library preparation protocols (such as Illumina RNA-Seq library preparations) have used methods similar to Gubler and Hoffman to produce blunt-end double-stranded cDNA amenable to adapter addition.

The Gubler and Hoffman procedure was designed for efficient full-transcript-length double-stranded cDNA with blunt ends for easy ligation into cloning vectors to enable further study using methods available in the 1980s. The goals of many next generation sequencing (NGS) library preparations (such as Illumina RNA-Seq library preparation) are different, however, and a fragmented representation of RNA molecules (rather than a single long cDNA) is required to enable efficient sequencing. Moreover, newer library construction methods such as tagmentation (such as Illumina DNA Flex PCR-Free (research use only, RUO) (Illumina) technology, previously known as Illumina's Nextera technology, and related products using Tn5 transposomes) do not require end-repaired molecules for adapter addition. As such, new ds-cDNA synthesis procedures that save on time and simplify workflows would be valuable for RNA library preparation protocols.

Faster conversion of RNA into a form compatible with library preparation is thus of high interest. A recent publication by Di et al., Proc. Natl. Acad. Sci. U.S.A. 117: 2886-2893 (2020) suggests RNA:DNA hybrids (the product of a ˜40-minute first strand cDNA synthesis) can be tagmented by Tn5 transposomes and rapidly converted into RNA sequencing libraries, but use of RNA:DNA hybrids may result in lower library yield.

The method described herein expedites conversion of single-stranded RNA samples into double-stranded cDNA. This method may be performed with a composition comprising a mixture of enzymes, including (1) a reverse transcriptase to prepare a first strand of cDNA and generate a DNA:RNA duplex, as well as (2) an RNA nickase (such as RNAse H) that can “nick” the RNA strand of this DNA:RNA duplex to allow for the RNA fragment to act as a primer to initiate synthesis of the second strand of cDNA.

These present methods may eliminate the need for a computer controlled programmable thermal cycler to reduce hands-on steps, improve total turnaround time, and simplify automation of library preparation from RNA samples. Further, these simplified methods may allow isothermal cDNA preparation, and mesophilic and thermostable compositions comprising enzymes are described. Some methods described herein allow cDNA preparation in a single 10-minute reaction performed at a single temperature (i.e., by an isothermal reaction). In addition, methods described herein allow library preparation from RNA samples with shorter incubation times and simpler workflows.

In some cases, the present methods of cDNA preparation use random primers (i.e., randomers) and omit steps of PCR-like amplification to avoid introduction of sequence-specific bias that may be seen with other methods, such as EP1929045. Further, certain RNA nickases, such as RNAse H, are known to randomly nick the RNA strand in a DNA:RNA duplex, and thus the step of nicking the RNA will also not introduce sequence-specific bias.

Applications for the present ds-cDNA and library preparation methods include disease surveillance and other assays for rapid quantitative identification of RNA molecules, where simplified workflows and easily automated procedures are highly desired. For example, workflows for applications such as enriching metagenomic RNA for viruses or pathogens of interest can be simplified by this procedure, making pathogen surveillance more amenable.

### SUMMARY

In accordance with the description, compositions and methods for preparing double-stranded complementary DNA (cDNA) are described herein. In some embodiments, these compositions and methods can allow for isothermal preparation of cDNA from RNA comprised in a sample. In some embodiments, compositions and methods allow for preparing a library of double-stranded DNA fragments from RNA comprised in the sample.

Embodiment 1. A composition for preparing double-stranded cDNA from RNA by an isothermal reaction comprising:
a. a reverse transcriptase;
b. an RNA nickase;
c. a DNA polymerase with strand displacement activity or 5′-3′ exonuclease activity; and
d. dNTPs.

Embodiment 2. The composition of embodiment 1, wherein the activity of the reverse transcriptase is greater than the activity of the RNA nickase.

Embodiment 3. The composition of embodiment 1 or embodiment 2, wherein the reverse transcriptase and the RNA nickase are comprised in a single enzyme.

Embodiment 4. The composition of any one of embodiments 1-3, wherein the reverse transcriptase and the DNA polymerase are comprised in a single enzyme with both RNA-dependent and DNA-dependent polymerase activity.

Embodiment 5. The composition of embodiment 4, wherein the single enzyme reduces competition between the reverse transcriptase and the DNA polymerase.

Embodiment 6. The composition of any one of embodiments 1-5, wherein the DNA polymerase has strand displacement activity.

Embodiment 7. The composition of any one of embodiments 1-6, wherein the DNA polymerase has 5′-3′ exonuclease activity.

Embodiment 8. The composition of any one of embodiments 1-7, wherein the reverse transcriptase is a polymerase with RNA-dependent DNA polymerase activity, optionally wherein the reverse transcriptase is Moloney Murine Leukemia Virus (MMLV) reverse transcriptase, a reverse transcriptase derived from a retrotransposon, or a Group II intron reverse transcriptase.

Embodiment 9. The composition of any one of embodiments 1-8, wherein the RNA nickase is RNAse H.

Embodiment 10. The composition of any one of embodiments 1-9, wherein the RNAse H is from Thermus thermophilus.

Embodiment 11. The composition of any one of embodiments 1-10, wherein the DNA polymerase is E. coli DNA polymerase I or Bst DNA polymerase.

Embodiment 12. The composition of any one of embodiments 1-11, wherein the reverse transcriptase, the RNA nickase, and/or the DNA polymerase are mesophilic enzymes.

Embodiment 13. The composition of embodiment 12, wherein the mesophilic enzymes have activity at 37° C.-49° C.

Embodiment 14. The composition of embodiment 13, wherein the mesophilic enzymes have activity at 37° C.

Embodiment 15. The composition of any one of embodiments 12-14, wherein the mesophilic reverse transcriptase is MMLV reverse transcriptase.

Embodiment 16. The composition of any one of embodiments 12-15, wherein the mesophilic RNA nickase is E. coli RNAse H.

Embodiment 17. The composition of any one of embodiments 12-16, wherein the mesophilic polymerase is E. coli DNA polymerase I.

Embodiment 18. The composition of any one of embodiments 1-11, wherein the reverse transcriptase, the RNA nickase, and/or the DNA polymerase are thermostable enzymes.

Embodiment 19. The composition of embodiment 18, wherein the thermostable enzymes have activity at 50° C.-72° C.

Embodiment 20. The composition of embodiment 19, wherein the thermostable enzymes have activity at 50° C.

Embodiment 21. The composition of any one of embodiments 18-20, wherein the thermostable reverse transcriptase is a thermostable variant of MMLV reverse transcriptase or a thermostable reverse transcriptase derived from a retrotransposon or a Group II intron reverse transcriptase.

Embodiment 22. The composition of any one of embodiments 18-21, wherein the thermostable RNA nickase is RNAse H from Thermus thermophilus.

Embodiment 23. The composition of any one of embodiments 18-22, wherein the thermostable DNA polymerase is Bst DNA polymerase.

Embodiment 24. The composition of any one of embodiments 1-23, wherein the RNA is bound to primers before preparing the double-stranded cDNA.

Embodiment 25. The composition of any one of embodiment 1-24, wherein the composition further comprises one or more additives chosen from DTT, BSA, Tris pH 7.5, KCl, and/or MgCl₂.

Embodiment 26. The composition of any one of embodiments 1-25, wherein the composition has a lower units/μl of the RNA nickase as compared to the units/μl of the reverse transcriptase and/or DNA polymerase.

Embodiment 27. The composition of any one of embodiments 1-26, wherein the composition further comprises an RNA nickase inhibitor.

Embodiment 28. The composition of embodiment 27, wherein the RNA nickase inhibitor lowers the activity of the RNA nickase.

Embodiment 29. The composition of any one of embodiments 1-28, wherein the units/μl of the RNA nickase and the DNA polymerase in the composition overlap.

Embodiment 30. The composition of any one of embodiments 1-29, wherein the activity of the DNA polymerase in the composition is 2-fold to 100-fold higher than the activity of the RNA nickase in the composition.

Embodiment 31. The composition of any one of embodiments 1-30, wherein the activity of the of the reverse transcriptase in the composition is 10-fold to 1,000-fold higher than the activity of the RNA nickase in the composition.

Embodiment 32. The composition of any one of embodiments 1-31, wherein the reverse transcriptase activity in the composition is 0.32 U/μl to 4.8 U/μl.

Embodiment 33. The composition of any one of embodiments 1-32, wherein the DNA polymerase activity in the composition is 0.04 U/μl to 0.37 U/μl.

Embodiment 34. The composition of any one of embodiments 1-33, wherein the RNA nickase activity in the composition is 0.004 U/μl to 0.04 U/μl.

Embodiment 35. The composition of any one of embodiments 1-33, wherein the RNA nickase activity in the composition is greater than 0.04 U/μl.

Embodiment 36. The composition of embodiment 35, wherein the RNA nickase activity in the composition is 0.05 U/μl to 0.3 U/μl.

Embodiment 37. A method of preparing double-stranded cDNA comprising:
a. combining primers with a sample comprising RNA and allowing binding of the primers to an RNA; and
b. combining the sample with the composition of any one of embodiments 1-36 and preparing double-stranded cDNA by an isothermal reaction.

Embodiment 38. The method of embodiment 37, wherein the primers comprise randomer primers.

Embodiment 39. The method of embodiment 37 or 38, wherein the primers comprise primers that bind specifically to a sequence comprised in the RNA.

Embodiment 40. The method of any one of embodiments 37-39, wherein the primers comprise hexamer primers.

Embodiment 41. The method of any one of embodiments 37-40, wherein the primers comprise primers comprising chemically modified nucleotides.

Embodiment 42. The method of embodiment 41, wherein the primers comprising chemically modified nucleotides render the RNA bound by the primers resistant to cleavage by the RNA nickase.

Embodiment 43. The method of embodiment 42, wherein the RNA nickase is RNAse H, and the RNA bound by the primers is resistant to cleavage by RNAse H.

Embodiment 44. The method of any one of embodiments 41-43, wherein the chemically modified nucleotides comprise methylphosphonate residues.

Embodiment 45. The method of embodiment 37-44, wherein the reverse transcriptase produces a first strand of cDNA.

Embodiment 46. The method of embodiment 45, wherein the reverse transcriptase produces a DNA:RNA duplex comprising the first strand of cDNA and a strand of RNA.

Embodiment 47. The method of embodiment 46, wherein the RNAse H nicks the RNA strand in the DNA:RNA duplex to produce RNA fragments.

Embodiment 48. The method of embodiment 47, wherein the DNA polymerase extends a second strand of DNA by priming from the RNA fragments.

Embodiment 49. The method of embodiment 47 or embodiment 48, wherein the RNA nickase and/or the 5′-3′ activity of the DNA polymerase removes the RNA fragments and 3′ RNA overhangs.

Embodiment 50. The method of any one of embodiments 37-49, wherein the DNA polymerase has 5′-3′ exonuclease activity and/or 3′-5′ exonuclease activity, wherein this activity produces blunt-ended double-stranded cDNA.

Embodiment 51. The method of any one of embodiments 37-50, wherein the dNTPs are used by both the reverse transcriptase and the DNA polymerase.

Embodiment 52. The method of any one of embodiments 37-51, wherein the isothermal reaction is at a temperature of from 30° C.-49° C.

Embodiment 53. The method of embodiment 52, wherein the isothermal reaction is at a temperature of 37° C.

Embodiment 54. The method of any one of embodiments 37-51, wherein the isothermal reaction is at a temperature of from 50° C.-72° C.

Embodiment 55. The method of embodiment 54, wherein the isothermal reaction is at a temperature of 50° C.

Embodiment 56. The method of embodiment 54 or embodiment 55, wherein the RNA exhibits a secondary structure that normally inhibits first strand synthesis at temperature below 50° C.

Embodiment 57. The method of any one of embodiments 37-56, wherein the rate of producing the first strand of cDNA by the reverse transcriptase is greater than the rate of nicking of the RNA by the RNA nickase.

Embodiment 58. The method of embodiment 57, wherein the activity of the reverse transcriptase exceeds the activity of the RNA nickase.

Embodiment 59. The method of any one of embodiments 37-58, wherein the isothermal reaction is incubated for 60 minutes or less, 45 minutes or less, 30 minutes or less, 20 minutes or less, 15 minutes of less, or 10 minutes or less.

Embodiment 60. The method of embodiment 59, wherein the isothermal reaction is incubated for 15 minutes or less.

Embodiment 61. The method of any one of embodiments 37-60, wherein incubations of at least 10 minutes, at least 20 minutes, at least 30 minutes, at least 45 minutes, or at least 60 minutes yield double-stranded cDNA for library preparation.

Embodiment 62. The method of any one of embodiments 37-61, further comprising performing off-target RNA depletion or mRNA enrichment with the sample comprising RNA before combining primers with the sample comprising RNA.

Embodiment 63. The method of embodiment 62, wherein the off-target RNA is ribosomal RNA.

Embodiment 64. The method of embodiment 62 or embodiment 63, wherein the mRNA enrichment comprises amplification with a poly-T primer or binding of mRNA to capture beads.

Embodiment 65. The method of embodiment 64, wherein the capture beads comprise a surface with capture oligonucleotides comprising poly-T sequences.

Embodiment 66. A composition for preparing a library of double-stranded cDNA fragments from RNA comprising:
a. a reverse transcriptase;
b. an RNA nickase;
c. a DNA polymerase with strand displacement activity or 5′-3′ exonuclease activity;
d. dNTPs; and
e. a transposome complex, wherein the transposome complex comprises:
   i. a transposase;
   ii. a first transposon comprising a transposon end sequence; and
   iii. a second transposon comprising a sequence fully or partially complementary to the transposon end sequence.

Embodiment 67. The composition of embodiment 66, wherein the composition further comprises Mg²⁺.

Embodiment 68. The composition of embodiment 67, wherein the Mg²⁺ concentration is 1 mM to 50 mM, optionally wherein the Mg²⁺ concentration is 5 mM to 20 mM, further optionally wherein the Mg2+ concentration is 8 mM.

Embodiment 69. The composition of any one of embodiments 66-68, wherein the library is prepared by an isothermal reaction.

Embodiment 70. The composition of any one of embodiments 66-69, wherein the RNA is bound to primers before preparing the library.

Embodiment 71. The composition of any one of embodiments 66-70, wherein the transposome complex is immobilized to a solid support.

Embodiment 72. The composition of embodiment 71, wherein the solid support is a bead.

Embodiment 73. The composition of embodiment 71 or embodiment 72, wherein the first transposon comprises an affinity element.

Embodiment 74. The composition of embodiment 73, wherein the affinity element is attached to the 5′ end of the first transposon.

Embodiment 75. The composition of embodiment 71 or 72, wherein the first transposon comprises a linker.

Embodiment 76. The composition of embodiment 75, wherein the linker has a first end attached to the 5′ end of the first transposon and a second end attached to an affinity element.

Embodiment 77. The composition of embodiment 71 or 72, wherein the second transposon comprises an affinity element.

Embodiment 78. The composition of embodiment 77, wherein the affinity element is attached to the 3′ end of the second transposon.

Embodiment 79. The composition of embodiment 71 or 72, wherein the second transposon comprises a linker.

Embodiment 80. The composition of embodiment 79, wherein the linker has a first end attached to the 3′ end of the second transposon and a second end attached to an affinity element.

Embodiment 81. The composition of any one of embodiments 73-74, 76-78, or 80, wherein the affinity element is biotin or dual biotin.

Embodiment 82. The composition of any one of embodiments 66-81, wherein the transposome complexes are present on the solid support at a density of at least 10³, 10⁴, 10⁵, or 10⁶ complexes per mm².

Embodiment 83. The composition of embodiment 66-82, wherein the first transposon further comprises one or more adapter sequences.

Embodiment 84. The composition of embodiment 83, wherein the first transposon comprises a 3′ transposon end sequence and a 5′ adapter sequence.

Embodiment 85. The composition of any one of embodiments 66-84, wherein the transposase is a Tn5 transposase.

Embodiment 86. The composition of embodiment 85, wherein the Tn5 transposase is hyperactive Tn5 transposase.

Embodiment 87. The composition of any one of embodiments 66-86, wherein the activity of the reverse transcriptase is greater than the activity of the RNA nickase.

Embodiment 88. The composition of any one of embodiments 66-87, wherein the reverse transcriptase and the RNA nickase are comprised in a single enzyme.

Embodiment 89. The composition of any one of embodiments 66-88, wherein the reverse transcriptase and the DNA polymerase are comprised in a single enzyme with both RNA-dependent and DNA-dependent polymerase activity.

Embodiment 90. The composition of embodiment 89, wherein the single enzyme reduces competition between the reverse transcriptase and the DNA polymerase.

Embodiment 91. The composition of any one of embodiments 66-90, wherein the DNA polymerase has strand displacement activity.

Embodiment 92. The composition of any one of embodiments 66-91, wherein the DNA polymerase has 5′-3′ exonuclease activity.

Embodiment 93. The composition of any one of embodiments 66-92, wherein the reverse transcriptase is a polymerase with RNA-dependent DNA polymerase activity, optionally wherein the reverse transcriptase is MMLV reverse transcriptase, a reverse transcriptase derived from a retrotransposon, or a Group II intron reverse transcriptase.

Embodiment 94. The composition of any one of embodiments 66-93, wherein the RNA nickase is RNAse H.

Embodiment 95. The composition of any one of embodiments 66-94, wherein the DNA polymerase is E. coli DNA polymerase I.

Embodiment 96. The composition of any one of embodiments 66-95, wherein the reverse transcriptase, the RNA nickase, and/or the DNA polymerase are mesophilic enzymes.

Embodiment 97. The composition of embodiment 96, wherein the mesophilic enzymes have activity at 37° C.-49° C.

Embodiment 98. The composition of embodiment 97, wherein the mesophilic enzymes have activity at 37° C.

Embodiment 99. The composition of any one of embodiments 96-98, wherein the mesophilic reverse transcriptase is MMLV reverse transcriptase.

Embodiment 100. The composition of any one of embodiments 96-99, wherein the mesophilic RNA nickase is E. coli RNAse H.

Embodiment 101. The composition of any one of embodiments 96-100, wherein the mesophilic polymerase is E. coli DNA polymerase I.

Embodiment 102. The composition of any one of embodiments 66-95, wherein the reverse transcriptase, the RNA nickase, and/or the DNA polymerase are thermostable enzymes.

Embodiment 103. The composition of embodiment 102, wherein the thermostable enzymes have activity at 50° C.-72° C.

Embodiment 104. The composition of embodiment 103, wherein the thermostable enzymes have activity at 50° C.

Embodiment 105. The composition of any one of embodiments 102-104, wherein the thermostable reverse transcriptase is a thermostable variant of MMLV reverse transcriptase or a thermostable reverse transcriptase derived from a retrotransposon or a Group II intron reverse transcriptase.

Embodiment 106. The composition of any one of embodiments 102-105, wherein the thermostable RNA nickase is RNAse H from Thermus thermophilus.

Embodiment 107. The composition of any one of embodiments 102-106, wherein the thermostable DNA polymerase is Bst DNA polymerase.

Embodiment 108. The composition of any one of embodiments 102-107, wherein (1) the reverse transcriptase, the RNA nickase, and/or the DNA polymerase are thermostable enzymes and (2) the Mg²⁺ concentration is 1 mM to 50 mM, optionally wherein the Mg²⁺ concentration is 5 mM to 20 mM, further optionally wherein the Mg²⁺ concentration is 8 mM.

Embodiment 109. A method of preparing a library of double-stranded cDNA fragments comprising:
a. combining primers with a sample comprising RNA and allowing binding of the primers to an RNA; and
b. combining the sample with the composition of any one of embodiments 66-108 and (i) preparing double-stranded cDNA by an isothermal reaction and (ii) preparing double-stranded cDNA fragments.

Embodiment 110. The method of embodiment 109, wherein solid-phase reversible immobilization purification is not performed between preparing double-stranded cDNA by an isothermal reaction and preparing double-stranded cDNA fragments.

Embodiment 111. The method of embodiment 109 or 110, wherein the combining primers with a sample and the combining the sample with the composition of any one of embodiments 66-108 are performed in the same step.

Embodiment 112. The method of any one of embodiments 109-111, wherein (i) preparing double-stranded cDNA and (ii) preparing double-stranded cDNA fragments are both performed by a single isothermal reaction.

Embodiment 113. The method of embodiment 109-111, wherein (i) preparing double-stranded cDNA and (ii) preparing double-stranded cDNA fragments are performed at different temperatures.

Embodiment 114. The method of any one of embodiments 109-113, wherein the (i) preparing double-stranded cDNA and (ii) preparing double-stranded cDNA fragments are performed in a single reaction vessel.

Embodiment 115. The method of any one of embodiments 109-114, wherein the combining primers with a sample comprising RNA comprises mixing the sample comprising RNA with an elution, primer, and fragmentation mix.

Embodiment 116. The method of any one of embodiments 109-115, wherein the combining primers with a sample comprising RNA is performed at 55° C. or higher.

Embodiment 117. The method of embodiment 109-116, wherein the combining primers with a sample comprising RNA is performed at 65° C.

Embodiment 118. The method of any one of embodiments 109-117, wherein the primers comprise randomer primers.

Embodiment 119. The method of any one of embodiments 109-118, wherein the primers comprise primers that bind specifically to a sequence comprised in the RNA.

Embodiment 120. The method of any one of embodiments 109-119, wherein the primers comprise hexamer primers.

Embodiment 121. The method of any one of embodiments 109-120, wherein the primers comprise primers comprising chemically modified nucleotides.

Embodiment 122. The method of embodiment 121, wherein the primers comprising chemically modified nucleotides render the RNA bound by the primers resistant to cleavage by the RNA nickase.

Embodiment 123. The method of embodiment 122, wherein the RNA nickase is RNAse H, and the RNA bound by the primers is resistant to cleavage by RNAse H.

Embodiment 124. The method of any one of embodiments 121-123, wherein the chemically modified nucleotides comprise methylphosphonate residues.

Embodiment 125. The method of embodiment 109-124, wherein the reverse transcriptase produces a first strand of cDNA.

Embodiment 126. The method of embodiment 125, wherein the reverse transcriptase produces a DNA:RNA duplex comprising the first strand of cDNA and a strand of RNA.

Embodiment 127. The method of embodiment 126, wherein the RNAse H nicks the RNA strand in the DNA:RNA duplex to produce RNA fragments.

Embodiment 128. The method of embodiment 127, wherein the DNA polymerase extends a second strand of DNA by priming from the RNA fragments.

Embodiment 129. The method of embodiment 127 or embodiment 128, wherein the RNA nickase and/or the 5′-3′ activity of the DNA polymerase removes the RNA fragments and 3′ RNA overhangs.

Embodiment 130. The method of any one of embodiments 109-129, wherein the DNA polymerase has 5′-3′ and/or 3′-5′ exonuclease activity, wherein this activity produces blunt-ended double-stranded cDNA.

Embodiment 131. The method of any one of embodiments 109-130, wherein the dNTPs are used by both the reverse transcriptase and the DNA polymerase.

Embodiment 132. The method of any one of embodiments 109-131, wherein the isothermal reaction for preparing double-stranded cDNA is at a temperature of from 30° C.-49° C.

Embodiment 133. The method of embodiment 132, wherein the isothermal reaction for preparing double-stranded cDNA is at a temperature of 37° C. or above.

Embodiment 134. The method of embodiment 133, wherein the isothermal reaction for preparing double-stranded cDNA is at a temperature of 37° C.

Embodiment 135. The method of embodiment 133, wherein the isothermal reaction for preparing double-stranded cDNA is at a temperature of 55° C.

Embodiment 136. The method of embodiment 134, wherein (i) preparing double-stranded cDNA and (ii) preparing double-stranded cDNA fragments are both performed by a single isothermal reaction at 37° C.

Embodiment 137. The method of embodiment 133, wherein preparing double-stranded cDNA and/or preparing double-stranded cDNA fragments are performed above

Embodiment 138. The method of embodiment 137, wherein preparing double-stranded cDNA fragments is performed at 55° C.

Embodiment 139. The method of embodiment 138, wherein the preparing double-stranded cDNA fragments is performed for 30 minutes or less or 15 minutes or less.

Embodiment 140. The method of embodiment 138 or 139, wherein preparing double-stranded cDNA is performed at 37° C. and preparing double-stranded cDNA fragments is performed at 55° C.

Embodiment 141. The method of any one of embodiments 109-140, wherein the Mg²⁺ concentration of the composition used for the method is 1 mM to 50 mM, optionally wherein the Mg²⁺ concentration is 5 mM to 20 mM, further optionally wherein the Mg²⁺ concentration is 8 mM.

Embodiment 142. The method of any one of embodiments 109-141, wherein the rate of producing the first strand of cDNA by the reverse transcriptase is greater than the rate of nicking of the RNA by the RNA nickase.

Embodiment 143. The method of any one of embodiments 109-142, wherein the activity of the reverse transcriptase exceeds the activity of the RNA nickase.

Embodiment 144. The method of any one of embodiments 109-143, wherein (i) preparing double-stranded cDNA by an isothermal reaction and (ii) preparing double-stranded cDNA fragments are performed with a total incubation of 60 minutes or less or 30 minutes or less.

Embodiment 145. The method of any one of embodiments 109-144, further comprising performing off-target RNA depletion or mRNA enrichment with the sample comprising RNA before combining primers with the sample comprising RNA.

Embodiment 146. The method of embodiment 145, wherein the off-target RNA is ribosomal RNA.

Embodiment 147. The method of embodiment 145 or 146, wherein the mRNA enrichment comprises amplification with a poly-T primer or binding of mRNA to capture beads.

Embodiment 148. The method of embodiment 147, wherein the capture beads comprise a surface with capture oligonucleotides comprising poly-T sequences.

Embodiment 149. The method of any one of embodiments 109-148, wherein the preparing double-stranded cDNA fragments is performed with enrichment.

Embodiment 150. The method of embodiment 149, wherein the enrichment is performed with hybrid capture.

Embodiment 151. The method of embodiment 150, wherein the hybrid capture is performed with target-specific biotinylated probes.

Embodiment 152. The method of embodiment 151, wherein the target-specific biotinylated probes bind to sequences from one or more infectious diseases.

Embodiment 153. The method of embodiment 152, wherein the one or more infectious diseases comprises one or more respiratory viruses.

Embodiment 154. The method of any one of embodiments 109-153, wherein the method further comprises amplifying the double-stranded cDNA fragments to prepare amplicons.

Embodiment 155. The method of embodiment 154, wherein the amplifying is performed with target-specific primers.

Embodiment 156. The method of embodiment 155, wherein the target-specific primers bind sequences from one or more infectious diseases.

Embodiment 157. The method of embodiment 156, wherein the one or more infectious diseases comprises one or more respiratory viruses.

Embodiment 158. The method of any one of embodiments 154-157, wherein the amplicons are subjected to solid-phase reversible immobilization purification.

Embodiment 159. The method of embodiment 158, wherein the total reaction time from combining primers with a sample comprising RNA until purification of amplicons is 2 hours or less, 2.5 hours or less, or 3 hours or less.

Embodiment 160. The method of any one of embodiments 109-159, wherein the first transposon comprises a modified transposon end sequence comprising a mosaic end sequence, wherein the mosaic end sequence comprises one or more mutations as compared to a wild-type mosaic end sequence, wherein the mutation comprises a substitution with
a. a uracil;
b. an inosine;
c. a ribose;
d. an 8-oxoguanine;
e. a thymine glycol;
f. a modified purine; or
g. a modified pyrimidine.

Embodiment 161. The method of embodiment 160, wherein the wild-type mosaic end sequence comprises SEQ ID No: 1, and further wherein the one or more mutations comprise a substitution at A16, C17, A18, and/or G19.

Embodiment 162. The method of embodiment 161, wherein:
a. the substitution at A16 is A16T, A16C, A16G, A16U, A16Inosine, A16Ribose, A16-8-oxoguanine, A16Thymine glycol, A16Modified purine, or A16Modified pyrimidine;
b. the substitution at C17 is C17T, C17A, C17G, C17U, C17Inosine, C17Ribose, C17-8-oxoguanine, C17Thymine glycol, C17Modified purine, or C17Modified pyrimidine;
c. the substitution at A18 is A18G, A18T, A18C, A18U, A18Inosine, A18Ribose, A18-8-oxoguanine, A18Thymine glycol, A18Modified purine, or A18Modified pyrimidine; and/or
d. the substitution at G19 is G19T, G19C, G19A, G19U, G19Inosine, G19Ribose, G19-8-oxoguanine, G19Thymine glycol, G19Modified purine, or G19Modified pyrimidine.

Embodiment 163. The method of any one of embodiments 160-162, further comprising:
a. combining the double-stranded cDNA fragments with (1) an endonuclease or (2) a combination of a DNA glycosylase and heat, basic conditions, or an endonuclease/lyase that recognizes abasic sites and cleaving the first transposon end at the uracil, inosine, ribose, 8-oxoguanine, thymine glycol, modified purine, and/or modified pyrimidine within the mosaic end sequence to remove all or part of the first transposon end from the fragments; and
b. ligating an adapter onto the 5′ and/or 3′ ends of the fragments.

Embodiment 164. The method of embodiment 163, wherein the modified purine is 3-methyladenine or 7-methylguanine.

Embodiment 165. The method of embodiment 163, wherein the modified pyrimidine is 5-methylcytosine, 5-formylcytosine, or 5-carboxycytosine.

Embodiment 166. The method of any one of embodiments 163-165, wherein the all or part of the first transposon end that is cleaved is partitioned away from the rest of the sample.

Embodiment 167. The method of any one of embodiments 163-166, further comprising filling in the 3′ ends of the fragments and phosphorylating the 3′ ends of fragments with a kinase before ligating.

Embodiment 168. The method of embodiment 167, wherein the filling in is performed with T4 DNA polymerase.

Embodiment 169. The method of embodiment 168, further comprising adding a single A overhang to the 3′ end of the fragments.

Embodiment 170. The method of embodiment 169, wherein a polymerase adds the single A overhang.

Embodiment 171. The method of embodiment 170, wherein the polymerase is (i) Taq or (ii) Klenow fragment, exo-.

Embodiment 172. The method of any one of embodiments 163-171, wherein the fragments comprise 0-3 bases of the mosaic end sequence.

Embodiment 173. The method of any one of embodiments 163-172, further comprising sequencing the fragments after ligating the adapter.

Embodiment 174. The method of embodiment 173, wherein the method does not require amplification of fragments before sequencing.

Embodiment 175. The method of embodiment 174, wherein fragments are amplified before sequencing.

Embodiment 176. The method of any one of embodiments 163-175, wherein the modified transposon end sequence comprises a uracil and the combination of a DNA glycosylase and an endonuclease/lyase that recognizes abasic sites is a uracil-specific excision reagent (USER).

Embodiment 177. The method of embodiment 176, wherein the USER is a mixture of uracil DNA glycosylase and endonuclease VIII or endonuclease III.

Embodiment 178. The method of any one of embodiments 163-175, wherein the modified transposon end sequence comprises an inosine and the endonuclease is endonuclease V.

Embodiment 179. The method of any one of embodiments 163-175, wherein the modified transposon end sequence comprises a ribose and the endonuclease is RNAse HII.

Embodiment 180. The method of any one of embodiments 163-175, wherein the modified transposon end sequence comprises a 8-oxoguanine and the endonuclease is formamidopyrimidine-DNA glycosylase (FPG) or oxoguanine glycosylase (OGG).

Embodiment 181. The method of any one of embodiments 163-175, wherein the modified transposon end sequence comprises a thymine glycol and the DNA glycosylase is endonuclease EndoIII (Nth) or Endo VIII.

Embodiment 182. The method of any one of embodiments 163-175, wherein the modified transposon end sequence comprises a modified purine and the DNA glycosylase is human 3-alkyladenine DNA glycosylase and the endonuclease is endonuclease III or VIII.

Embodiment 183. The method of embodiment 182, wherein the modified purine is 3-methyladenine or 7-methylguanine.

Embodiment 184. The method of any one of embodiments 163-175, wherein the modified transposon end sequence comprises a modified pyrimidine and:
a. the DNA glycosylase is thymine-DNA glycosylase (TDG) or mammalian DNA glycosylase-methyl-CpG binding domain protein 4 (MBD4) and the endonuclease/lyase that recognizes abasic sites is the endonuclease is endonuclease III or VIII; or
b. the endonuclease is DNA glycosylase/lyase ROS1 (ROS1).

Embodiment 185. The method of embodiment 184, wherein the modified pyrimidine is 5-methylcytosine, 5-formylcytosine, or 5-carboxycytosine.

Embodiment 186. The method of any one of embodiments 163-175, wherein the first transposon comprises a modified transposon end sequence comprising more than one mutation chosen from a uracil, an inosine, a ribose, 8-oxoguanine, a thymine glycol, a modified purine, or a modified pyrimidine and the (1) an endonuclease or (2) a combination of a DNA glycosylase and heat, basic conditions, or an endonuclease/lyase that recognizes abasic sites is an enzyme mixture.

Embodiment 187. The method of embodiment 186, wherein the modified purine is 3-methyladenine or 7-methylguanine.

Embodiment 188. The method of embodiment 186, wherein the modified pyrimidine is 5-methylcytosine, 5-formylcytosine, or 5-carboxycytosine.

Embodiment 189. The method of any one of embodiments 172-188, wherein cleaving the first transposon end generates a sticky end for ligating the adapter.

Embodiment 190. The method of embodiment 189, wherein the sticky end is longer than one base.

Embodiment 191. The method of any one of embodiments 163-190, wherein the adapter comprises a double-stranded adapter.

Embodiment 192. The method of any one of embodiments 163-191, wherein adapters are added to the 5′ and 3′ end of fragments.

Embodiment 193. The method of embodiment 192, wherein the adapters added to the 5′ and 3′ end of the fragments are different.

Embodiment 194. The method of any one of embodiments 163-193, wherein the adapter comprises a unique molecular identifier (UMI), primer sequence, anchor sequence, universal sequence, spacer region, index sequence, capture sequence, barcode sequence, cleavage sequence, sequencing-related sequence, and combinations thereof.

Embodiment 195. The method of any one of embodiments 163-194, wherein the adapter comprises a UMI.

Embodiment 196. The method of embodiment 195, wherein an adapter comprising a UMI is ligated to both the 3′ and 5′ end of fragments.

Embodiment 197. The method of any one of embodiments 163-196, wherein the adapter is a forked adapter.

Embodiment 198. The method of any one of embodiments 163-197, wherein the ligating is performed with a DNA ligase.

Embodiment 199. The method of any one of embodiments 109-198, wherein a stop tagmentation buffer is added after preparing double-stranded cDNA fragments.

Embodiment 200. The method of any one of embodiment 109-199, wherein the prepared double-stranded cDNA fragments are purified.

Embodiment 201. The method of any one of embodiments 109-200, wherein the double-stranded cDNA fragments are sequenced.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 provides the time and steps of cDNA synthesis in conventional protocols (such as, for example, Illumina RNA Preparation with Enrichment as described RNA Prep with Enrichment (L) Tagmentation Reference Guide, Document #1000000124435v02, Illumina, 2020 (“Document 1000000124435”)) as compared to the present method. X-axis reflects time scale, manual user intervention for addition of reagents required at start of each step, depicted by horizontal bars. Temperature profile shown as small horizontal lines below each method.

FIG. 2 shows a comparator cDNA synthesis protocol similar to the method of Gubler and Huffman, 1983, wherein first strand cDNA synthesis is temporally separated from second strand cDNA synthesis to generate ds-cDNA copies of the originating RNA.

FIG. 3 shows an overview of the present method of near-simultaneous isothermal generation of double-stranded cDNA from RNA and proposed mechanisms of action.

FIG. 4 shows the percentage of duplicate reads performance of a single-step protocol (1-step, labeled as “Present Method”) and standard procedure (in this example, Illumina RNA Prep with Enrichment, as described in Illumina document 470-2020-001-A (2020)). Black dots represent replicates of 10-, 20-, 30-, 45-, and 60-minute incubations of the single-step method.

FIG. 5 shows insert size performance of the single-step isothermal protocol (1-step, labeled as “Present Method”) and standard Illumina RNA Prep with Enrichment procedure. Black dots represent replicates of 10-, 20-, 30-, 45-, or 60-minute incubations with the single-step method.

FIG. 6 shows median coefficient of variance (CV) of coverage performance of the single-step isothermal protocol (1-step, labeled as “Present method”) and standard Illumina RNA Prep with Enrichment procedure. Black dots represent replicates of 10-, 20-, 30-, 45-, and 60-minute incubations of the single-step method.

FIGS. 7A and 7B shows fragments per kilobase per million mapped reads (FPKM) scatterplots. (A) Technical replicate correlation for the present ‘single-step’ method with 20 minutes incubation. Both libraries were generated with 12 ng of a mixture of Universal Human Reference RNA (UHR, Agilent PN 740000) and genomic RNA derived from bacteriophage MS2 (MS2, Roche PN 10165948001, GenBank accession NC 001417.2) at 80% UHR/20% MS2. (B) Comparison of a library prepared from 10 ng UHR by the Illumina RNA Prep with Enrichment method to a library prepared from 12 ng of a mixture of 80% UHR/20% MS2 by the single-step method.

FIG. 8 shows boxplots of FPKM R² values. FPKM comparison includes only genes targeted by the TruSight® RNA Pan-Cancer Panel kit panel. Left panel: single-step method comparisons between different incubation times. Middle panel: comparison between Illumina RNA Prep with Enrichment libraries and single-step method libraries. Right panel: replicate-to-replicate comparison of Illumina RNA Prep with Enrichment libraries performed during DVT. Key denotes RNA input, x-axis denotes incubation time of RNA method (in minutes), if applicable. NA=standard cDNA procedures and incubation times used in Project Illumina RNA Prep with Enrichment.

FIG. 9 shows Integrated Genomics Viewer (IGV) visualization of normalized read coverage across MS2 for the standard method released in Project Illumina RNA Prep with Enrichment (top track), a 20-minute single-step procedure (middle track), and a 10-minute single-step procedure (bottom track). Tracks in the lower frame represent percentage of G and C (% GC) in 24 bp windows (black=high % GC, white=low % GC), a well-characterized short hairpin sequence in MS2, and the protein-coding regions of MS2 (MS2 genome).

FIG. 10 shows density plots of the read-depth normalized per-base coverage of the MS2 in the single-step (top) and Illumina RNA Prep with Enrichment (bottom) cDNA preparation protocols. Note that the single-step protocol has a wider distribution and more extreme values, consistent with the larger CV of coverage presented in Table 2.

FIG. 11 shows primary alignment and performance metrics for 6 replicates of the thermostable formulation of present method.

FIGS. 12A and 12B show thermostable formulation performance. cDNA was prepared using the thermostable formulation, tagmented with enrichment bead-linked transposons or BLTs (eBLT), enriched with the TruSight® RNA Fusion Panel (as described in TruSight RNA® Fusion Panel Protocol Guide, Illumina Document #1000000009155 v00(2016)), and evaluated for reproducibility by an FPKM analysis. (A) Replicate correlation. (B) Comparison to the standard protocol released with Illumina RNA Prep with Enrichment. R² value is shown.

FIG. 13 shows a summary of a 1-pot method for combined cDNA synthesis and tagmentation, wherein a library of double-stranded DNA fragments is prepared in a single reaction vessel from a sample comprising RNA. The summary shows that the reaction temperature may optionally be increased to 55° C. at the end of an incubation to improve tagmentation efficiency.

FIGS. 14A-14C shows summaries of exemplary double-stranded cDNA preparations (using the present method) and 1-pot tagmentation preparations (combined cDNA and library fragment preparation). (A) Representative mesophilic and thermostable preparations. (B) Summaries of method of double-stranded cDNA synthesis with either 2-step or present method. (C) Comparison of different methods including tagmentation to prepare library fragments. EPH3=elution, primer, fragmentation mix; FSA=first strand synthesis actinomycin D mix; FSM=first strand synthesis mix; SMM=second strand mix; ST2=stop tagmentation buffer 2; SPRI=solid-phase reversible immobilization purification.

FIG. 15 shows library results with 2-step cDNA preparation followed by tagmentation with either BLTs (such as those comprised in Illumina® DNA Prep, (S) Tagmentation kit or comprised in Illumina® RNA Prep, (L) Tagmentation kit).

FIGS. 16A-16D show library results using different types of cDNA preparation and tagmentation reactions using a sample with 100 ng RNA. (A) Results with a 2-step cDNA preparation (standard method) followed by separate tagmentation reaction. (B) Results with a 1-step cDNA preparation (present method) followed by separate tagmentation reaction. (C) Results with 1-pot combined cDNA and tagmentation preparation. (D) Overlay of results under different conditions. All conditions resulted in preparation of a library of fragments.

FIGS. 17A-17C show results with a 1-pot combined cDNA preparation and tagmentation reaction. (A) Results of reaction in presence of reverse transcriptase (RT). (B) Results of no template control reaction (NTC, no RNA comprised in starting sample). (C) Results of reaction in absence of RT. As expected, fragments were only produced under conditions of (A), wherein the reaction comprised RNA template and RT.

FIGS. 18A-18B shows comparison of results with BLTs with a 1-pot combined cDNA preparation and tagmentation reaction for either 100 ng (A) or 10 ng (B) starting RNA.

FIGS. 19A-19B show results using ribosomal RNA (rRNA) depleted UHR libraries with different cDNA preparation and BLT tagmentation protocols. (A) Results with 1:10 diluted samples prepared with 2-step cDNA preparation followed by separate tagmentation reaction or 1-step cDNA preparation (present method) followed by separate tagmentation reaction. (B) Results with undiluted samples using a 1-pot combined cDNA and tagmentation preparation and control with no reverse transcriptase (No RVT).

FIGS. 20A-20C shown alignment and general performance metrics of different preparations. (A) Percentage aligned are all approximately 94-95%. (B) Median CV increased for combined reactions. (C) Percentage duplicates are higher for combined reactions. 2st=2-step cDNA preparation followed by separate tagmentation reaction; 1stC=1-step cDNA preparation (present method) followed by separate tagmentation reaction; 1potLib=1-pot combined cDNA and tagmentation preparation.

FIGS. 21A-21B show insert length (A) and alignment distribution (B) after library preparation under different conditions, using the same sample naming as in FIGS. 20A-20C.

FIGS. 22A-22C gene expression correlations between different reaction conditions. (A) Correlation between 2-step cDNA preparation (2 step) versus 1-pot cDNA preparation (1-pot) for 100 ng starting RNA. (B) Correlation between 2-step and 1-pot for 10 ng starting RNA. (C) Correlation between 1-step cDNA preparation followed by separate tagmentation reaction (1-step cDNA) versus 1-pot combined cDNA and tagmentation library preparation for 100 ng starting RNA.

FIG. 23 shows 10× gene coverage for 2st, 1stC, and 1potLib preparations with either 100 ng or 10 ng starting RNA. Sample naming is the same as in FIGS. 20A-20C.

FIG. 24 shows 5′ to 3′ read distribution for 2st, 1stC, and 1potLib preparations with 10 ng starting RNA. Sample naming is the same as in FIGS. 20A-20C.

FIG. 25 shows a comparison of the time and steps for 2-step cDNA followed by tagmentation (standard library preparation (std LP)), 1-step cDNA preparation followed by separate tagmentation reaction (1-step cDNA+tagmentation), and 1-pot combined cDNA and tagmentation library preparation. The 1-pot combined protocol can reduce library prep (LP) time by about 50%, to approximately 2 hours. The 1-pot combined protocol also may only have a single “clean up” step (such as using SPRI beads.)

FIG. 26 shows results from a variety of different library preparation (LP) protocols with no (NoTC), 1k (1kTC), 10k (10kTC), or 100k (100kTC) Twist control (TC, Twist Control Synthetic SARS-CoV-2 RNA Control 2 (#102024 Control 2*MN908947.3 Wuhan-Hu-1) from Twist Bioscience). “A” and “B” samples for each group refer to two separate samples. Enrichment was performed using the Respiratory Virus Oligos Panel Version 1 (Illumina) under conditions as summarized below:
Standard LP with enrichment (i.e., 2-step cDNA preparation followed by tagmentation as control, StdLP group)
1-Pot LP: 37° C. for 1 hr (4 mM Mg²⁺)—conditions outlined in Example 5 (1Pot group)
1-Pot LP: 37° C. for 45 minutes, followed by 55° C. for 15 min (55C group)
1-Pot LP: 37° C. for 1 hour, increase Mg²⁺ to 8 mM final (Mg group)
1-Pot LP: 37° C. for 1 hour, skipping washes and addition of ST2 stop tagmentation solution comprising SDS (NoST2 group).

FIG. 27 shows median coverage of Twist control at 1 million (1 M) reads for the different protocols outlined in FIG. 26. Results was performed using enrichment with Respiratory Virus Oligos Panel Version 1 (Illumina). Arrows show that the best performance was seen with the control (standard library prep) and with 1-pot LP with increased Mg²⁺ to 8 mM final (Mg group).

FIG. 28 shows results from a variety of conditions of library preparation (LP). The tested groups included some combined protocols that had incubations at 55° C. and a 8 mM Mg²⁺ concentration, as well as a 1-Pot LP at 37° C. with 8 mM Mg²⁺. These 1-Pot protocols were compared to a standard library preparation (Std LP) with 2-step cDNA preparation followed by tagmentation.

FIG. 29 show results of coverage for Twist controls with different incubation times using enrichment with Respiratory Virus Oligos Panel Version 2 (Illumina). 37-1 hr=37° for 1 hour; 45 min+55C=37° for 45 minutes and 55° for 15 minutes; 30 min+55C=37° for 30 minutes and 55° for 15 minutes; 14 min+55C=37° for 15 minutes and 55° for 15 minutes.

FIGS. 30A and 30B show results on experiments with rRNA depleted samples. (A) Percentage of duplicates for 2st, CTL, 1stC, and 1potLib samples either with 4 mM Mg²⁺ for 1 hour at 37° C. (left groups) or with 8 mM Mg²⁺ for 1 hour at 37° C. and at 55° C. for 15 minutes (3715) or for 30 minutes (3730). Conditions for the 10ngCTL and 2st groups were the same with a separate first and second strand reaction to prepare cDNA, clean-up, and then tagmentation with a BLT. (B) Number of genes detected with different protocols.

### DESCRIPTION OF THE SEQUENCES

The table below provides a listing of certain sequences referenced herein.

Description of the Sequences

| | | SEQ ID |
|---|---|---|
| Description | Sequences | NO |
| Mosaic end (ME) | AGATGTGTATAAGAGACAG | 1 |
| sequence (transferred | | |
| strand) | | |
| Outside end (OE) | CTGACTCTTATACACAAGT | 2 |
| Inside end (IE) | CTGTCTCTTGATCAGATCT | 3 |
| Mosaic end (ME) | CTGTCTCTTATACACATCT | 4 |
| (non-transferred | | |
| strand) | | |
| U16 transferred strand | AGATGTGTATAAGAGUCAG | 5 |
| (TS), Modified ME | | |
| with A16U substitution | | |
| (transferred strand, | | |
| substitution in bold) | | |
| Modified ME (non- | TCTACACATATTCTCAGTC | 6 |
| transferred strand) | | |
| presented in 3′-5′ | | |
| orientation) with T16A | | |
| substitution (in bold) | | |
| U17 TS, Modified ME | AGATGTGTATAAGAGAUAG | 7 |
| with C17U substitution | | |
| (transferred strand, | | |
| substitution in bold) | | |
| Modified ME′ (non- | TCTACACATATTCTCTATC | 8 |
| transferred strand, | | |
| presented in 3′-5′ | | |
| orientation) with G17A | | |
| substitution (in bold) | | |
| U18 TS, Modified ME | AGATGTGTATAAGAGACUG | 9 |
| with A18U substitution | | |
| (transferred strand, | | |
| substitution in bold) | | |
| Modified ME′ (non- | TCTACACATATTCTCTGAC | 10 |
| transferred strand, | | |
| presented in 3′-5′ | | |
| orientation) with T18A | | |
| substitution (in bold) | | |
| A14 sequence | TCGTCGGCAGCGTC | 11 |
| B15 sequence | GTCTCGTGGGCTCGG | 12 |
| P5 | AATGATACGGCGACCACCGAGAUCTACAC | 13 |
| P7 | CAAGCAGAAGACGGCATACGAGAT | 14 |
| Biotinylated ME′ | /5Phos/CTGTCTCTTATACACATCT/3BiotinN/ | 15 |
| (non-transferred | | |
| strand) | | |
| I19 TS, Modified ME | AGATGTGTATAAGAGACAI | 16 |
| with G19I substitution | | |
| (transferred strand, | | |
| substitution in bold) | | |
| U19 TS Modified ME | AGATGTGTATAAGAGACAU | 17 |
| with G19U substitution | | |
| (transferred strand, | | |
| substitution in bold) | | |
| O16 TS, Modified ME | AGATGTGTATAAGAG/i8oxodG/CAG | 18 |
| with A16O substitution | | |
| (transferred strand, | | |
| substitution in bold) | | |
| O17 TS, Modified ME | AGATGTGTATAAGAGA/i8oxodG/AG | 19 |
| with C17O substitution | | |
| (transferred strand, | | |
| substitution in bold) | | |
| O18 TS, Modified ME | AGATGTGTATAAGAGAC/i8oxodG/G | 20 |
| with A18O substitution | | |
| (transferred strand, | | |
| substitution in bold) | | |
| O19 TS Modified ME | AGATGTGTATAAGAGACA/38oxodG/ | 21 |
| with G19O substitution | | |
| (transferred strand, | | |
| substitution in bold) | | |
| I16 TS, Modified ME | AGATGTGTATAAGAGICAG | 22 |
| with A16I substitution | | |
| (transferred strand, | | |
| substitution in bold) | | |
| I17 TS, Modified ME | AGATGTGTATAAGAGAIAG | 23 |
| with C17I substitution | | |
| (transferred strand, | | |
| substitution in bold) | | |
| I18 TS, Modified ME | AGATGTGTATAAGAGACIG | 24 |
| with A18I substitution | | |
| (transferred strand, | | |
| substitution in bold) | | |

### DESCRIPTION OF THE EMBODIMENTS

I. Compositions for Preparing Double-Stranded cDNA from RNA by an Isothermal Reaction

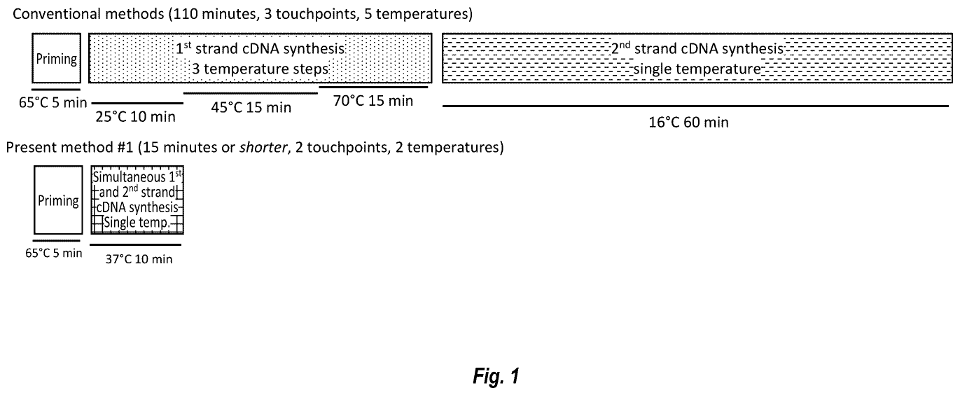

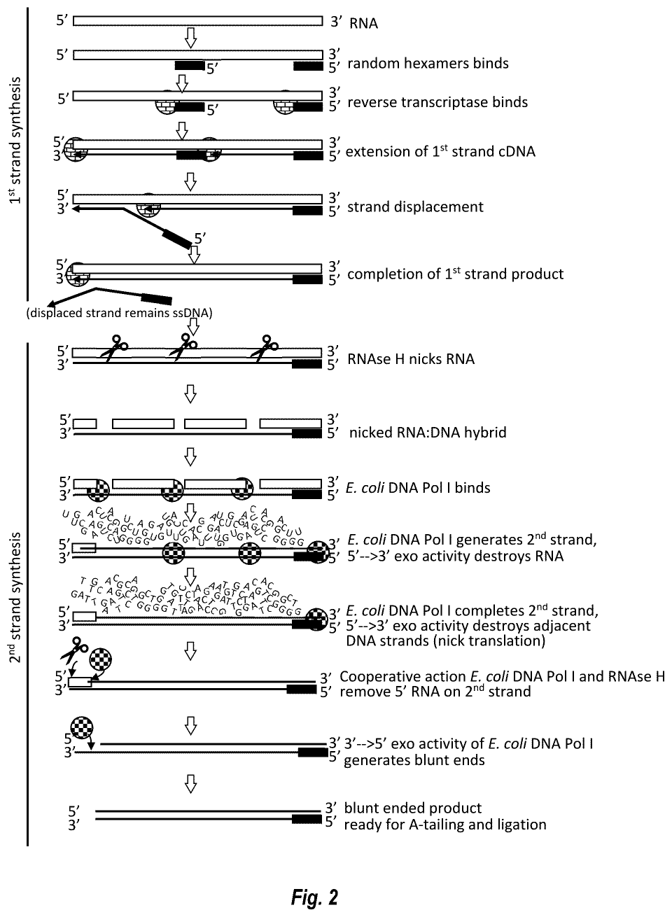

As described herein, a specialized mix of enzymes can achieve efficient ds-cDNA conversion that is suitable for Illumina library preparation technologies, such as tagmentation by Tn5 (e.g. Illumina DNA Flex PCR-Free technology and bead-linked transposomes). This method can be performed in a single step as short as 10 minutes at a single temperature. Such a preparation of cDNA in a single step may be referred to as a 1-step cDNA method (also described herein as the “present method” of cDNA preparation). Formulations with mesophilic (˜37° C.) and thermostable (˜50° C.) enzymes are possible. The advantages in shorter time versus conventional methods is shown in FIG. 1 (where the process of converting RNA into ds-cDNA is reduced from 110 minutes with conventional models to 15 minutes with the present method). Potential applications of the present methods are fast infectious disease surveillance and fast RNA and DNA co-assays for genotyping.

In some embodiments, the method of cDNA preparation does not amplify the nucleic acid content and instead converts RNA to double-stranded cDNA. In some embodiments, the method does not comprise a step of PCR amplification or a PCR-like process to prepare multiple amplicons from a given sequence comprised in the RNA.

In some embodiments, RNA is bound to primers before preparing double-stranded cDNA.

In some embodiments, the composition is for preparation of ds-cDNA by an isothermal reaction. As used herein, an “isothermal reaction” refers to a reaction conducted at substantially constant temperature, i.e., without varying the reaction temperature in which the enzyme reaction occurs by more than 15% from a baseline temperature. As such, an isothermal reaction is conducted at a constant temperature or with changes from a baseline from temperature of 15% or less. In some further embodiments, the reaction has even less temperature variation and may be conducted at a temperature with changes from baseline of 10% or less, or 5% of less. In some embodiments, the reaction is conducted without a temperature change. In some embodiments, a reaction can be performed isothermally without need for computer-controlled temperature modulation in a thermal cycler. In some instances of a reaction that can be performed isothermally without the need for computer-controlled modulation in a thermal cycler the temperature may be 37° C. or 50° C. In some embodiments, a primer has been bound to RNA before a composition is added to a sample comprising RNA. In some embodiments, the reaction is a mesophilic reaction or a thermostable reaction.

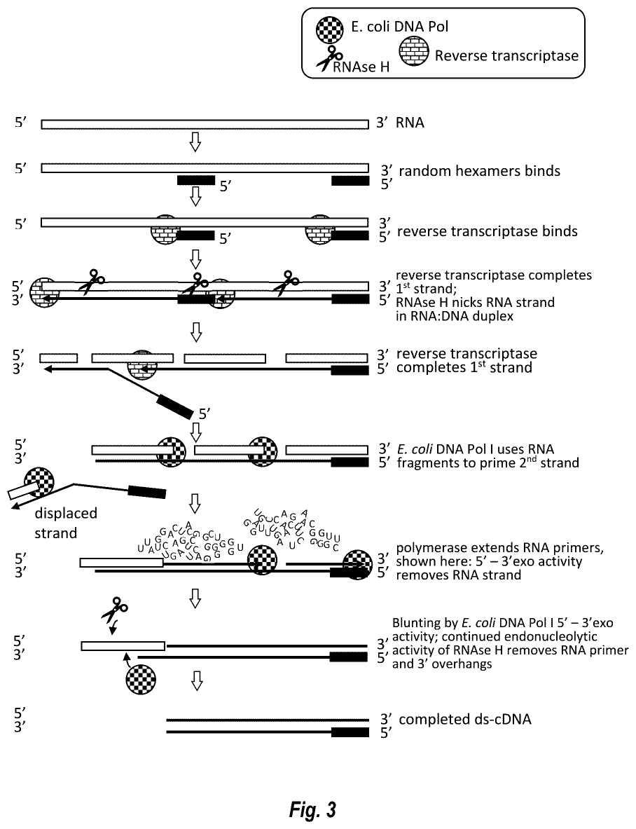

In some embodiments, a composition for preparing double-stranded cDNA from RNA by an isothermal reaction comprises (i) a reverse transcriptase; (ii) an RNA nickase; (iii) a DNA polymerase with strand displacement activity or 5′-3′ exonuclease activity; and (iv) dNTPs. Such a composition may be used in methods of ds-cDNA as described below and in FIG. 3. In some embodiments, the formulation of enzymes in the composition has a coordinated action to generate ds-cDNA amenable to tagmentation (for example, by bead-linked transposomes (BLTs) and Illumina DNA Flex PCR-Free (research use only, RUO) technology products).

In some embodiments, a composition further comprises an RNA nickase inhibitor. In some embodiments, the RNA nickase inhibitor is an RNAse inhibitor.

In some embodiments, the reverse transcriptase can bind to primers that are bound to RNA prior to addition of the composition to a sample comprising RNA. In some embodiments, the reverse transcriptase can generate a first strand of cDNA from RNA.

In some embodiments, the RNA nickase can nick RNA, which may be termed an “RNA nickase.” In some embodiments, the RNA nickase is a ribonuclease. In some embodiments, the ribonuclease is RNAse H. Accordingly, an RNA nickase may be referred to as “an enzyme with RNAse-H-like activity,” since RNAse is a representative RNA nickase.

In some embodiments, the RNA nickase is comprised in a reverse transcriptase that has RNAse H activity. While many commercially available reverse transcriptases have been engineered to lack RNAse H activity (as this may improve cDNA synthesis yields), many nonengineered reverse transcriptases have RNAse H activity.

In some embodiments, the RNA nickase can nick RNA comprised in an RNA:DNA hybrid. In some embodiments, the RNA nickase can nick a strand of RNA hybridized to a first strand of cDNA. For example, RNase H is reported to digest RNA from a DNA:RNA hybrid approximately every 7-21 bases (Schultz et al., J. Biol. Chem. 2006, 281:1943-1955; Champoux and Schultz, FEBS 1 2009, 276:1506-1516). In some embodiments, RNA fragments generated by the RNA nickase can be used to prime a second strand of cDNA off a first strand of cDNA.

In some embodiments, RNAse H can nick the RNA strand in an RNA:DNA hybrid, and a DNA polymerase can then use the 3′ OH end of the nicked RNA fragment as a primer to initiate synthesis of a second cDNA strand. Such a process cannot be performed with degraded or fragmented RNA prepared in other ways, as the RNA fragments would have a 3′ phosphate, and a polymerase cannot use such ends to prime synthesis of a second cDNA strand.

In some embodiments, an RNA nickase can also remove RNA primers and 3′ overhangs after generation of a second strand of cDNA.

In some embodiments, the activity of the reverse transcriptase is greater than the activity of the RNA nickase. In some embodiments, the generating of a first strand of cDNA by the reverse transcriptase is faster than the rate of nicking of the RNA by the RNA nickase. In this way, a first strand of cDNA can be generated before the RNA template is nicked. In some embodiments, the reverse transcriptase and the RNA nickase are comprised in a single enzyme. In some embodiments, a single enzyme comprising a reverse transcriptase and an RNA nickase is Avian Myeloblastosis Virus Reverse Transcriptase (AMV RT), Moloney Murine Leukemia Virus (MMLV Reverse Transcriptase), or a Group II intron reverse transcriptase. Alternatively, MMLV without RNA nickase activity, or with severely reduced RNA nickase activity, may be used in some embodiments.

In some embodiments, the reverse transcriptase and the DNA polymerase are comprised in a single enzyme with both RNA-dependent and DNA-dependent polymerase activity. In some embodiments, this single enzyme reduces competition between the reverse transcriptase and the DNA polymerase. In some embodiments, a single enzyme comprising both RNA-dependent and DNA-dependent polymerase activity eliminates steric inhibition of DNA polymerase binding by the reverse transcriptase.

In some embodiments, the DNA polymerase mediates second strand cDNA synthesis. In some embodiments, the DNA polymerase destroys the RNA strand.

In some embodiments, the DNA polymerase has strand displacement activity. In some embodiments, the DNA polymerase can displace a first strand of cDNA that has been generated.

In some embodiments, the DNA polymerase has 5′ to 3′ exonuclease activity. In some embodiments, the DNA polymerase can remove RNA by its 5′ to 3′ exonuclease activity. In some embodiments, the DNA polymerase can produce blunt-ended ds-cDNA by its 5′ to 3′ exonuclease activity and/or 3′ to 5′ exonuclease activity.

In some embodiments, the reverse transcriptase is a polymerase with RNA-dependent DNA polymerase activity. Any such reverse transcriptase that is a polymerase with RNA-dependent DNA polymerase activity may be used, and one skilled in the art would be aware of a wide variety of such enzymes. In some embodiments, the reverse transcriptase is MMLV reverse transcriptase or a reverse transcriptase derived from a retrotransposon or a Group II intron reverse transcriptase. In some embodiments, the reverse transcriptase is Protoscript II (New England Biolabs (NEB)), a recombinant MMLV reverse transcriptase with limited RNAse H activity and increased thermostability.

In some embodiments, the RNA nickase is RNAse H. In some embodiments, the RNAse H is from Thermus thermophilus. In some embodiments, the DNA polymerase is E. coli DNA polymerase I or Bst DNA polymerase.

In some embodiments, a composition comprises one or more additives besides enzymes and dNTPs. In some embodiments, a composition comprises one or more additives chosen from dithiothreitol (DTT), bovine serum albumin (BSA), Tris pH 7.5, KCl, and/or MgCl₂. In some embodiments, a composition comprises DTT and BSA. One skilled in the art would be well-aware that such additives may improve function of various enzyme compositions and analysis of different additives is comprised in regular assay development. Accordingly, this list of representative additives only serves as an example and one skilled in the art could exclude or substitute for such additives.

In some embodiments, the composition is for an isothermal mesophilic reaction or an isothermal thermostable reaction.

A. Compositions for Mesophilic Ds-cDNA Preparation

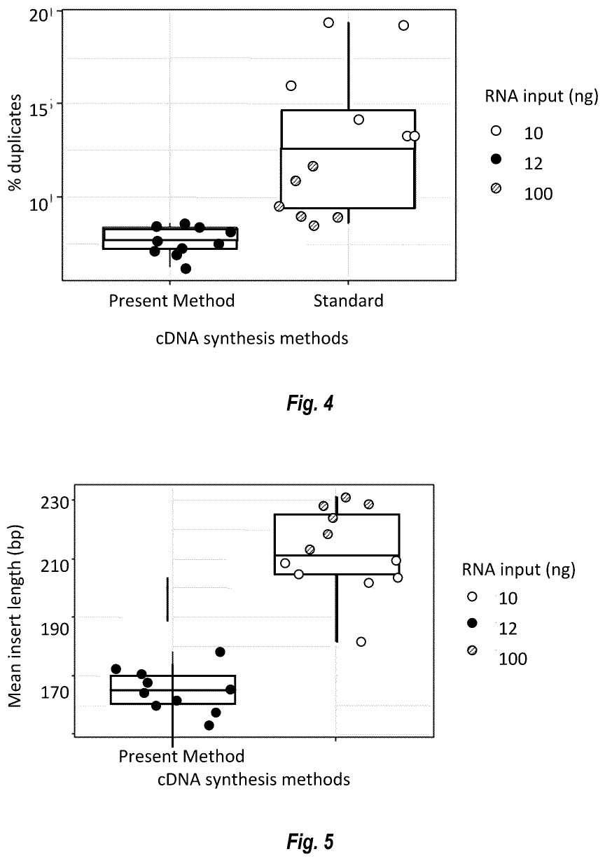

In some embodiments, the composition may be for preparing ds-cDNA by an isothermal mesophilic reaction. As used herein, a mesophilic reaction refers to a reaction performed at 37° C.-49° C. In some embodiments, a mesophilic reaction can be performed isothermally 37° C. without need for computer-controlled temperature modulation in a thermal cycler. Data in FIGS. 4-10 show results using compositions for mesophilic ds-DNA preparation in a 1-step process (i.e., present method).

In some embodiments, the reverse transcriptase, the RNA nickase, and/or the DNA polymerase are mesophilic enzymes. In some embodiments, the mesophilic enzymes have activity at 37° C.-49° C. In some embodiments, the mesophilic enzymes have activity at 37° C.

In some embodiments, the mesophilic reverse transcriptase is MMLV reverse transcriptase. In some embodiments, the mesophilic RNA nickase is E. coli RNAse H. In some embodiments, the mesophilic polymerase is E. coli DNA polymerase I.
Table 1 provides representative enzymes comprised in a composition for mesophilic ds-cDNA preparation.

TABLE 1
Representative enzymes comprised in a composition
for mesophilic ds-cDNA preparation

| | | Temperature |
|---|---|---|
| Enzyme | Role | optima |
| Reverse transcriptase | Synthesis of 1^{st} strand cDNA | 42° C.-48° C. |
| (MMLV) | | |
| E. coli RNAse H | Nicks RNA:DNA hybrid | ~37° C. |
| E. coli DNA | 2^{nd} strand cDNA synthesis, | ~37° C. |
| Polymerase I | destruction of RNA strand | |

B. Compositions for Thermostable Ds-cDNA Preparation

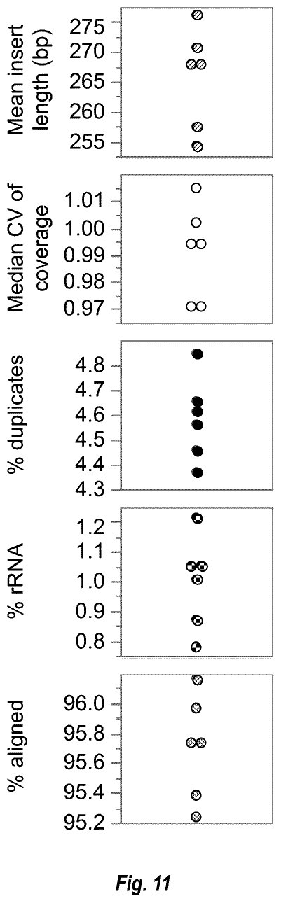

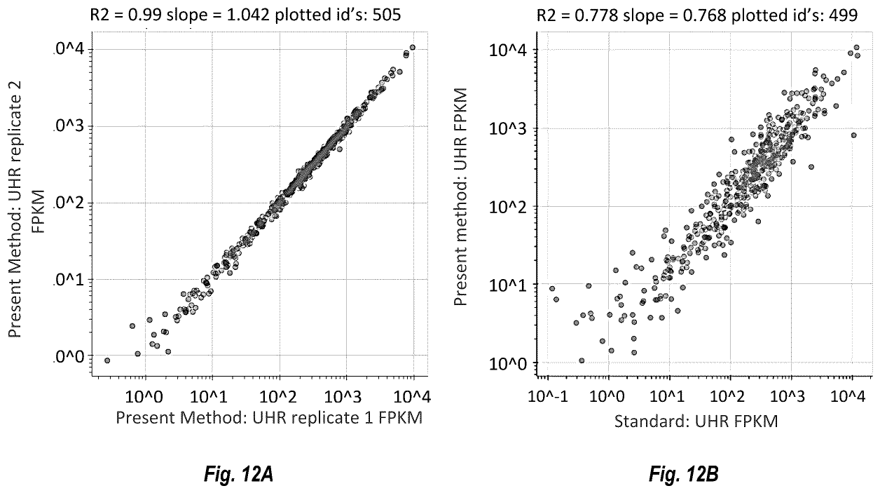

In some embodiments, the composition may be for preparing ds-cDNA by an isothermal thermostable reaction. As used herein, a thermostable reaction refers to a reaction performed at 50° C. or above. In some embodiments, a thermostable reaction can be performed isothermally at 50° C. or above without need for computer-controlled temperature modulation in a thermal cycler. Data in FIGS. 11-12B show results using compositions for thermostable ds-DNA preparation in a 1-step process (i.e., present method).

In some embodiments, the reverse transcriptase, the RNA nickase, and/or the DNA polymerase are thermostable enzymes. In some embodiments, the thermostable enzymes have activity at 50° C.-72° C. In some embodiments, the thermostable enzymes have activity at 50° C. In some embodiments, the thermostable reverse transcriptase is a thermostable variant of MMLV reverse transcriptase or a thermostable reverse transcriptase derived from a retrotransposon or a Group II intron reverse transcriptase. In some embodiments, the thermostable variant of MMLV reverse transcriptase is ProtoScript II (New England Biolabs). In some embodiments, the thermostable RNA nickase is RNAse H from Thermus thermophilus.

In some embodiments, the thermostable DNA polymerase is Bst DNA polymerase. In some embodiments, a DNA polymerase from a mesophilic composition is substituted with Bst DNA polymerase. In some embodiments, the Bst DNA polymerase is Bst 3.0 DNA polymerase (New England Biolabs).

Table 2 provides representative enzymes comprised in a composition for thermostable ds-cDNA preparation.

TABLE 2
Representative enzymes comprised in a composition
for thermostable ds-cDNA preparation

| | | | Mesophilic |
|---|---|---|---|
| Thermostable | Temperature | | counterpart to be |
| enzyme | optima | Role | replaced |
| Thermostable | 65° C.-95° C. | Nicking of | E. coli RNAse H |
| RNAse H | | RNA | |
| from Thermus | | | |
| thermophilus | | | |
| Thermostable | 42° C.-48° C. | 1^{st} strand | Use enzyme with a |
| MMLV reverse | | synthesis | higher temperature |
| transcriptase | | | tolerance |

C. Balance of Enzyme Units in a Composition

The present compositions improve double-stranded cDNA preparation in a single isothermal reaction using a balance of enzyme activity. For example, if the activity of RNase is relatively high compared to other enzymes in the composition, the RNA template (from the original sample) will be degraded before a sufficient amount of a first strand of cDNA is prepared. In such a scenario, little cDNA would be produced.

A composition with a proper balance of enzyme activity for a 1-step cDNA preparation (i.e., the present method) may be termed a “master mix.”

Accordingly, compositions with appropriate balances of enzyme activity improve yield of a double-stranded cDNA preparation.

Unit ranges of commercially available enzymes would be well-known to those skilled in the art and could be easily reviewed from supplier websites or technical documents provided with commercial enzymes. Table 3 provides information on certain enzymes and their characteristics from New England Biolabs (NEB), along with unit definitions. The present methods are not limited to these specific enzymes, but this table serves to provide information on the well-known characteristics of commercially available representative enzymes (such as that described for enzymes commercially available from NEB). One skilled in the art would be aware of a wide range of different enzymes that may be used in the present compositions and could select enzymes based on the desired conditions for performing methods described herein. In Table 3, RNAse H represents an RNA nickase.

TABLE 3
Characteristics of Certain Representative Enzymes

| Enzyme | Unit definition |
|---|---|
| RNAse H: | NEB RNase H: (5 U/μl) One unit is defined as the amount of enzyme that |
| | will hydrolyze 1 nmol of ribonucleotides from 20 pmol of a fluorescently |
| | labeled 50 base pair RNA-DNA hybrid in a total reaction volume of 50 μl in |
| | 20 minutes at 37° C. |
| DNA Pol I: | NEB E. coli DNA Polymerase I: (10 U/μl) One unit is defined as the amount |
| | of enzyme that will incorporate 10 nmol of dNTP into acid insoluble material |
| | in 30 minutes at 37° C. |
| RNAse | NEB: (40 U/μl) One unit is defined as the amount of RNase Inhibitor, |
| Inhibitor: | Murine required to inhibit the activity of 5 ng of RNase A by 50%. Activity is |
| | measured by the inhibition of hydrolysis of cytidine 2,3′-cyclic |
| | monophosphate by RNase A. |
| Reverse | NEB Protoscript II: (200 U/μl). One unit is defined as the amount of enzyme |
| Transcriptase | that will incorporate 1 nmol of dTTP into acid-insoluble material in a total |
| | reaction volume of 50 μl in 10 minutes at 37° C. using poly(rA)•oligo(dT)18 as |
| | template. |

In some embodiments, the correct balance of reverse transcriptase, RNA nickase, and DNA polymerase allows preparation of double-stranded cDNA from RNA using a composition described herein in a single isothermal reaction.

In some embodiments, the composition has a lower units/μl of the RNA nickase (such as RNAse H) as compared to the units/μl of the reverse transcriptase and/or DNA polymerase. In some embodiments, a composition comprises an RNA nickase inhibitor that serves to lower the activity of the RNA nickase. In some embodiments, an RNA nickase inhibitor is an RNAse inhibitor.

In some embodiments, a composition may comprise an RNAse inhibitor that functions to limit contamination by exogenous RNAse. In some embodiments, an RNAse inhibitor serves to inhibit RNAse A, which is a common laboratory contamination that does not function as an RNA nickase.

In some embodiments, the units/μl of the RNA nickase and the DNA polymerase in the composition overlap DNA polymerase. In some embodiments, the DNA polymerase has 2-fold to 100-fold higher activity than that of the RNA nickase.

In some embodiments, units/μl of the reverse transcriptase is 10-fold to 1,000-fold higher than that of the RNA nickase.

In some embodiments, the units/μl of the reverse transcriptase and the DNA polymerase in the composition overlap. In some embodiments, the units/μl of the reverse transcriptase in the composition is higher than that of the DNA polymerase.

In some embodiments, the reverse transcriptase activity in the composition is 0.32 U/μl to 4.8 U/μl. In some embodiments, the DNA polymerase activity in the composition is 0.04 U/μl to 0.37 U/μl. In some embodiments, the RNA nickase activity in the composition is 0.004 U/μl to 0.04 U/μl. In some embodiments, this RNA nickase is RNAse H.

In some embodiments, the RNA nickase activity in a thermostable composition is relatively higher than that of a mesophilic composition. In some embodiments, the activity of the RNA nickase in a thermostable composition is greater than 0.04 U/μl. In some embodiments, the activity of the RNA nickase in a thermostable composition is 0.05 U/μl-0.3 U/μl.

II. Compositions for Preparing a Library from RNA

As described herein, a specialized mix of enzymes can be used to prepare a library of double-stranded cDNA fragments from RNA in a sample. In some embodiments, a composition allows for preparation of a library of cDNA fragments in a single reaction vessel. In some embodiments, these fragments can be used for sequencing.

In some embodiments, a composition for a 1-pot library preparation (for combined cDNA and library preparation) may be a cDNA preparation “master mix” together with BLTs.

In some embodiments, a library is prepared by an isothermal reaction. In some embodiments, a single temperature is used for the reactions to prepare double-stranded cDNA and to prepare a library of cDNA fragments. Accordingly, any compositions used for preparing cDNA described herein may also be used for preparing a library of cDNA fragments, as long as a transposome complex is included in the composition.

In some embodiments, the RNA is bound to primers before preparing the library.

In some embodiments, a composition for preparing a library of double-stranded cDNA fragments from RNA comprises a reverse transcriptase; an RNA nickase; a DNA polymerase with strand displacement activity or 5′-3′ exonuclease activity; dNTPs; and a transposome complex. In some embodiments, the transposome complex comprises a transposase; a first transposon comprising a transposon end sequence; and a second transposon comprising a sequence fully or partially complementary to the transposon end sequence.

In some embodiments, the composition allows first and second stranded cDNA preparation, followed by fragmentation of this double-stranded DNA. In some embodiments, the composition comprises enzymes for mesophilic cDNA synthesis. In some embodiments, the composition comprises enzymes for thermostable cDNA synthesis.

In some embodiments, a composition for preparing a library comprises components described for a composition for preparing double-stranded cDNA and further comprises a transposome complex. In some embodiments, the transposome complex comprises a transposase; a first transposon comprising a transposon end sequence; and a second transposon comprising a sequence fully or partially complementary to the transposon end sequence. In some embodiments, the transposome complex allows generation of fragments of the double-stranded cDNA generated by preparing double-stranded cDNA from RNA.

In some embodiments, the cDNA may be fragmented by transposome complexes as the cDNA is prepared from RNA, without requiring a change in composition components or the reaction vessel. In some embodiments, the cDNA does not need to be purified before fragments of the cDNA are prepared using the present composition.

In some embodiments, a composition for preparing a library of double-stranded cDNA from RNA comprises magnesium. Magnesium is known to promote transposase activity (See Picelli et al., Genome Research 24:2033-2040 (2014)). In some embodiments, the Mg²⁺ concentration of a composition is 1 mM to 50 mM. In some embodiments, the Mg²⁺ concentration of a composition is 5 mM to 20 mM. In some embodiments, the Mg²⁺ concentration of a composition is 8 mM.

A. Transposome Complexes

Transposon based technology can be utilized for fragmenting DNA, wherein target nucleic acids, such as genomic DNA, are treated with transposome complexes that simultaneously fragment and tag (“tagmentation”) the target, thereby creating a population of fragmented nucleic acid molecules tagged with unique adaptor sequences at the ends of the fragments. Tagmentation includes the modification of DNA by a transposome complex comprising transposase enzyme complexed with one or more tags (such as adaptor sequences) comprising transposon end sequences (referred to herein as transposons). Tagmentation thus can result in the simultaneous fragmentation of the DNA and ligation of the adaptors to the 5′ ends of both strands of duplex fragments.

A transposition reaction is a reaction wherein one or more transposons are inserted into target nucleic acids at random sites or almost random sites. Components in a transposition reaction may include a transposase (or other enzyme capable of fragmenting and tagging a nucleic acid as described herein, such as an integrase) and a transposon element that includes a double-stranded transposon end sequence that binds to the enzyme, and an adaptor sequence attached to one of the two transposon end sequences. One strand of the double-stranded transposon end sequence is transferred to one strand of the target nucleic acid and the complementary transposon end sequence strand is not (i.e., a non-transferred transposon sequence). The adaptor sequence can comprise one or more functional sequences (e.g., primer sequences) as needed or desired.

A “transposome complex” is comprised of at least one transposase (or other enzyme as described herein) and a transposon recognition sequence. In some such systems, the transposase binds to a transposon recognition sequence to form a functional complex that is capable of catalyzing a transposition reaction. In some respects, the transposon recognition sequence is a double-stranded transposon end sequence. The transposase binds to a transposase recognition site in a target nucleic acid and inserts the transposon recognition sequence into a target nucleic acid. In some such insertion events, one strand of the transposon recognition sequence (or end sequence) is transferred into the target nucleic acid, resulting in a cleavage event. Exemplary transposition procedures and systems that can be readily adapted for use with the transposases.

A “transposase” means an enzyme that is capable of forming a functional complex with a transposon end-containing composition (e.g., transposons, transposon ends, transposon end compositions) and catalyzing insertion or transposition of the transposon end-containing composition into a double-stranded target nucleic acid. A transposase as presented herein can also include integrases from retrotransposons and retroviruses.

Exemplary transposases that can be used with certain embodiments provided herein include (or are encoded by): Tn5 transposase, Sleeping Beauty (SB) transposase, Vibrio harveyi, MuA transposase and a Mu transposase recognition site comprising R1 and R2 end sequences, Staphylococcus aureus Tn552, Ty1, Tn7 transposase, Tn/O and IS10, Mariner transposase, Tc1, P Element, Tn3, bacterial insertion sequences, retroviruses, and retrotransposon of yeast. More examples include IS5, Tn10, Tn903, IS911, and engineered versions of transposase family enzymes. The methods described herein could also include combinations of transposases, and not just a single transposase.

In some embodiments, the transposase is a Tn5, Tn7, MuA, or Vibrio harveyi transposase, or an active mutant thereof. In other embodiments, the transposase is a Tn5 transposase or a mutant thereof. In other embodiments, the transposase is a Tn5 transposase or a mutant thereof. In other embodiments, the transposase is a Tn5 transposase or an active mutant thereof. In some embodiments, the Tn5 transposase is a hyperactive Tn5 transposase, or an active mutant thereof. In some aspects, the Tn5 transposase is a Tn5 transposase as described in PCT Publ. No. WO2015/160895, which is incorporated herein by reference. In some aspects, the Tn5 transposase is a hyperactive Tn5 with mutations at positions 54, 56, 372, 212, 214, 251, and 338 relative to wild-type Tn5 transposase. In some aspects, the Tn5 transposase is a hyperactive Tn5 with the following mutations relative to wild-type Tn5 transposase: E54K, M56A, L372P, K212R, P214R, G251R, and A338V. In some embodiments, the Tn5 transposase is a fusion protein. In some embodiments, the Tn5 transposase fusion protein comprises a fused elongation factor Ts (Tsf) tag. In some embodiments, the Tn5 transposase is a hyperactive Tn5 transposase comprising mutations at amino acids 54, 56, and 372 relative to the wild type sequence. In some embodiments, the hyperactive Tn5 transposase is a fusion protein, optionally wherein the fused protein is elongation factor Ts (Tsf). In some embodiments, the recognition site is a Tn5-type transposase recognition site (Goryshin and Reznikoff, J. Biol. Chem., 273:7367, 1998). In one embodiment, a transposase recognition site that forms a complex with a hyperactive Tn5 transposase is used (e.g., EZ-Tn5TM Transposase, Epicentre Biotechnologies, Madison, Wis.). In some embodiments, the Tn5 transposase is a wild-type Tn5 transposase.

In some embodiments, the transposome complex comprises a dimer of two molecules of a transposase. In some embodiments, the transposome complex is a homodimer, wherein two molecules of a transposase are each bound to first and second transposons of the same type (e.g., the sequences of the two transposons bound to each monomer are the same, forming a “homodimer”). In some embodiments, the compositions and methods described herein employ two populations of transposome complexes. In some embodiments, the transposases in each population are the same. In some embodiments, the transposome complexes in each population are homodimers, wherein the first population has a first adaptor sequence in each monomer and the second population has a different adaptor sequence in each monomer.

The term “transposon end” refers to a double-stranded nucleic acid DNA that exhibits only the nucleotide sequences (the “transposon end sequences”) that are necessary to form the complex with the transposase or integrase enzyme that is functional in an in vitro transposition reaction. In some embodiments, a transposon end is capable of forming a functional complex with the transposase in a transposition reaction. As non-limiting examples, transposon ends can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US 2010/0120098, the content of which is incorporated herein by reference in its entirety. Transposon ends can comprise any nucleic acid or nucleic acid analogue suitable for forming a functional complex with the transposase or integrase enzyme in an in vitro transposition reaction. For example, the transposon end can comprise DNA, RNA, modified bases, non-natural bases, modified backbone, and can comprise nicks in one or both strands. Although the term “DNA” is used throughout the present disclosure in connection with the composition of transposon ends, it should be understood that any suitable nucleic acid or nucleic acid analogue can be utilized in a transposon end.

The term “transferred strand” refers to the transferred portion of both transposon ends. Similarly, the term “non-transferred strand” refers to the non-transferred portion of both “transposon ends.” The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction.

In some embodiments, the transferred strand and non-transferred strand are covalently joined. For example, in some embodiments, the transferred and non-transferred strand sequences are provided on a single oligonucleotide, e.g., in a hairpin configuration. As such, although the free end of the non-transferred strand is not joined to the target DNA directly by the transposition reaction, the non-transferred strand becomes attached to the DNA fragment indirectly, because the non-transferred strand is linked to the transferred strand by the loop of the hairpin structure. Additional examples of transposome structure and methods of preparing and using transposomes can be found in the disclosure of US 2010/0120098, the content of which is incorporated herein by reference in its entirety.

In some embodiments, transposome complexes are designed to incorporate unique molecular identifiers (UMIs) or index sequences. In some embodiments, transposome complexes comprise modified mosaic end sequences.

In some embodiments, a composition for library preparation is optimized for increasing library yield. In some embodiments, the composition increases yield by comprising thermostable enzymes such that an incubation above 37° C. can be performed to increase tagmentation yield, such as an incubation at 55° C. In some embodiments, the composition comprises 8 mM or more Mg²⁺ to increase tagmentation yield. In some embodiments, (1) the reverse transcriptase, the RNA nickase, and/or the DNA polymerase are thermostable enzymes and (2) the Mg²⁺ concentration is 1 mM to 50 mM, optionally wherein the Mg²⁺ concentration is 5 mM to 20 mM, further optionally wherein the Mg²⁺ concentration is 8 mM. In some embodiments, a composition comprises thermostable enzymes and 8 mM or more Mg²⁺.

B. Transposomes for Incorporating UMIs or Index Sequences

In some embodiments, the first transposon further comprises UMI or index sequences that are incorporated into fragments when preparing the double-stranded cDNA fragments.

Unique molecular identifiers (UMIs) are sequences of nucleotides applied to or identified in nucleic acid molecules that may be used to distinguish individual nucleic acid molecules from one another. UMIs may be sequenced along with the nucleic acid molecules with which they are associated to determine whether the read sequences are those of one source nucleic acid molecule or another. The term “UMI” may be used herein to refer to both the sequence information of a polynucleotide and the physical polynucleotide per se. UMIs are similar to barcodes, which are commonly used to distinguish reads of one sample from reads of other samples, but UMIs are instead used to distinguish nucleic acid template fragments from another when many fragments from an individual sample are sequenced together. UMIs may be defined in many ways, such as described in WO 2019/108972 and WO 2018/136248, which are incorporated herein by reference.

Unique molecular identifiers (UMIs) are sequences of nucleotides applied to or identified in nucleic acid molecules that may be used to distinguish individual nucleic acid molecules from one another. UMIs may be sequenced along with the nucleic acid molecules with which they are associated to determine whether the read sequences are those of one source nucleic acid molecule or another. The term “UMI” may be used herein to refer to both the sequence information of a polynucleotide and the physical polynucleotide per se. UMIs are similar to bar codes, which are commonly used to distinguish reads of one sample from reads of other samples, but UMIs are instead used to distinguish nucleic acid template fragments from another when many fragments from an individual sample are sequenced together. UMIs may be defined in many ways, such as described in WO 2019/108972 and WO 2018/136248, which are incorporated herein by reference.

Unique molecular identifiers (UMIs) are sequences of nucleotides applied to or identified in nucleic acid molecules that may be used to distinguish individual nucleic acid molecules from one another. UMIs may be sequenced along with the nucleic acid molecules with which they are associated to determine whether the read sequences are those of one source nucleic acid molecule or another. The term “UMI” may be used herein to refer to both the sequence information of a polynucleotide and the physical polynucleotide per se. UMIs are similar to bar codes, which are commonly used to distinguish reads of one sample from reads of other samples, but UMIs are instead used to distinguish nucleic acid template fragments from another when many fragments from an individual sample are sequenced together. UMIs may be defined in many ways, such as described in WO 2019/108972 and WO 2018/136248, which are incorporated herein by reference.

In some embodiments, the library of UMIs comprises nonrandom sequences. In some embodiments, nonrandom UMIs (nrUMIs) are predefined for a particular experiment or application. In certain embodiments, rules are used to generate sequences for a set or select a sample from the set to obtain a nrUMI. For instance, the sequences of a set may be generated such that the sequences have a particular pattern or patterns. In some implementations, each sequence differs from every other sequence in the set by a particular number of (e.g., 2, 3, or 4) nucleotides. That is, no nrUMI sequence can be converted to any other available nrUMI sequence by replacing fewer than the particular number of nucleotides. In some implementations, a set of UMIs used in a sequencing process includes fewer than all possible UMIs given a particular sequence length. For instance, a set of nrUMIs having 6 nucleotides may include a total of 96 different sequences, instead of a total of 4^{A}6=4096 possible different sequences. In some embodiments, the library of UMIs comprises 120 nonrandom sequences.

In some implementations where nrUMIs are selected from a set with fewer than all possible different sequences, the number of nrUMIs is fewer, sometimes significantly so, than the number of source DNA molecules. In such implementations, nrUMI information may be combined with other information, such as virtual UMIs, read locations on a reference sequence, and/or sequence information of reads, to identify sequence reads deriving from a same source DNA molecule.

In some embodiments, the library of UMIs may comprise random UMIs (rUMIs) that are selected as a random sample, with or without replacement, from a set of UMIs consisting of all possible different oligonucleotide sequences given one or more sequence lengths. For instance, if each UMI in the set of UMIs has n nucleotides, then the set includes 4ⁿ UMIs having sequences that are different from each other. A random sample selected from the 4^{A}n UMIs constitutes a rUMI.

In some embodiments, the library of UMIs is pseudo-random or partially random, which may comprise a mixture of nrUMIs and rUMIs.

In some embodiments, adapter sequences or other nucleotide sequences may be present between the UMI and the insert DNA.

In some embodiments, adapter sequences or other nucleotide sequences may be present between each UMI and the insert DNA.

In some embodiments, the UMI is located 3′ of the insert DNA. In some embodiments, a sequence of nucleic acids representing one or more adapter sequences may be located between the UMI and the insert DNA.

In some embodiments, UMIs are added to target double stranded nucleic acids using oligonucleotides or polynucleotides during or after tagmentation of said nucleic acids. In many embodiments, UMIs are added to target double stranded nucleic acids before a library amplification step.

In some embodiments, UMI reagents from the TruSight® Oncology workflow (Illumina Catalog #20024586) may be utilized in accordance with the present disclosure.

In some embodiments, the double stranded nucleic acid molecules in a UMI library each comprises one unique UMI sequence, or single UMI. In some embodiments, the UMI may be located on either side of the insert DNA. In some embodiments, adapter sequences or other nucleotide sequences may be present between the UMI and the insert DNA.

In some embodiments, the UMI library comprises duplex UMI, which may lower the limit of error detection as compared to the use of a single UMI. Duplex UMIs enable a skilled artisan to pair a plus strand with its minus strand despite errors that may arise in a sequencing reaction. Such sequencing mismatches are identified during sequencing, and the sequence of a nucleic acid fragment can still be correctly reconstituted despite having mismatches. In some embodiments, a method of producing a UMI library comprising duplex UMI comprises forked adapters. In some embodiments, the forked adapters are BLT fork adapters.

In some embodiments, each double-stranded nucleic acid fragment in the UMI library comprises two, three or four UMI sequences. The UMI sequences may have complementary sequences with each other or may each have a different sequence.

In some embodiments, adapter sequences or other nucleotide sequences may be present between each UMI and the insert DNA.

In some embodiments, the UMI is located 5′ of the insert DNA. In some embodiments, the UMI is located 3′ of the insert DNA. In some embodiments, a sequence of nucleic acids representing one or more adapter sequences may be located between the UMI and the insert DNA. In some embodiments, the UMI is located between an adapter sequence and a transposon end sequence

In some embodiments, the UMI can be on the first strand, second strand, or both strands of the double-stranded target nucleic acid fragments. In some embodiments, the UMI is on the first strand. In some embodiments, a first copy of the UMI is on the first strand and a second copy of the UMI is on the second strand of the double-stranded target nucleic acid fragments. In some embodiments, a first UMI is on a first strand and a second UMI is on a second strand.

1. In-Line UMIs and Index Sequences

A UMI may be located anywhere on a double stranded nucleic acid molecule. In many embodiments, the location of a UMI on a double stranded nucleic acid molecule will vary. In some embodiments, the UMI is located directly adjacent to the insert DNA, i.e., the UMI is an “in-line UMI.” In some embodiments, the in-line UMI is adjacent to the 3′ end of the insert DNA. In some embodiments, the in-line UMI is adjacent to the 5′ end of the insert DNA.

While UMIs are useful for removing PCR duplicates in double-stranded nucleic acids and for detection of low-frequency variants, UDIs are useful for mitigating sample misassignment due to index hopping in library sequencing and demultiplexing. UDIs are unique i5 and i7 index sequences that are added to the ends of target nucleic acids so that both ends contain a UDI. UDIs are used with patterned flow cells, such as Illumina's NovaSeq 6000 system (See, e.g., WO 2018/204423, WO 2018/208699, WO 201/9055715, and WO 2016/176091; which are incorporated by reference herein in their entireties). One skilled in the art would appreciate that in-line UMIs allow for the compatibility of UMI libraries with standard, downstream library preparations that utilize UDIs, such as sample multiplexing PCR and sequencing chemistry recipes in Illumina's TruSeq™ and AmpliSeg™ workflows. In some embodiments, the sequencing methods used with in-line UMIs do not require custom primers or custom reads.

In some embodiments, a standard sequencing method is used to sequence a UMI library with in-line UMIs. In these embodiments, the UMI is adjacent to the 3′ end of the insert nucleic acids. As such, each UMI and insert nucleic acid sequence is captured using a standard sequencing primer without having to sequence additional sequence in between them.

In some embodiments, the “in-line UMI” is located between the insert DNA and an adapter sequence. In some embodiments, the adapter sequence is a second adapter sequence.

C. Transposomes for Incorporating Modified Transposon Ends with Mutations in the Mosaic End Sequence

Described herein are modified transposon end sequences comprising a mosaic end sequences, including those disclosed in US Application Nos. 63/224,201, 63/167,150, and PCT/US22/22167, each of which is incorporated herein in its entirety. In some embodiments, these modified transposon end sequences comprise a mosaic end sequence that allows for cleavage and removal of the mosaic end sequence after transposition. A critical requirement for transposition is the “mosaic end” (ME) which is specifically recognized by Tn5 and required for its transposition activity. Tn5 natively recognizes the “outside end” (OE) and “inside end” (IE) sequences (as shown in Table 4), which have been shown to be highly intolerant to mutations, with most mutations leading to decreased activity (See J. C. Makris et al. PNAS 85(7):2224-28 (1988)). Later work demonstrated that a chimeric sequence derived from IE and OE, termed the “mosaic end” (Table 4), along with a mutant Tn5 enzyme, increased the transposition activity approximately 100-fold relative to the native system (See Maggie Zhou et al., Journal of Molecular Biology 276(5): 913-25 (1998)). This hyperactive system is used in Illumina's Illumina DNA Flex PCR-Free (RUO) products. Crystal structures of Tn5 in complex with DNA substrates indicate that 13 of the 19 basepairs have nucleobase-specific crystal contacts (See Douglas R. Davies et al., Science 289 5476:77-85 (2000)), while other bases have been shown to play a role in catalysis (See Mindy Steiniger-White et al., Journal of Molecular Biology 322(5): 971-82 (2002)). Typically, activity of Tn5 has been assessed by in vivo reporter systems (papillation assays, described in Zhou et al. J. Mol. Biol. 276:913-925 (1998)).

TABLE 4
Known DNA substrates of Tn5 transposase

| Substrate | Sequence | SEQ ID NO |
|---|---|---|
| Outside End (OE) | CTG [symbol] CTCTT [symbol] CA [symbol] T | 2 |
| Inside End (IE) | CTGTCTCTTGATCAGATC | 3 |
| | T | |
| Mosaic End (ME) | CTGTCTCTT [symbol] CA [symbol] TCT | 4 |

In Table 4, sequences in normal font indicate shared sequences, sequences in italics with double-underline are derived from the native OE substrate, and sequences in bold italics are derived from the native IE substrate.

A representative wild-type mosaic end sequence (transferred strand) is SEQ ID NO: 1. A variety of mutant Tn5 and transposon ends are described in WO 2015160895 and U.S. Pat. No. 9,080,211, each of which are incorporated by reference in their entirety herein, and may be appropriate for use in the methods described herein.

Several DNA enzymes or enzyme combinations can mediate the selective removal of modified bases such as uracil, inosine, ribose bases, 8-oxo G, thymine glycol, modified purines, and modified pyrimidines among others (See Table 5 and Properties of DNA Repair Enzymes and Structure-specific Endonucleases, New England Biolabs, downloaded Jan. 20, 2022, from www.international.neb.com/tools-and-resources/selection-charts and Jacobs and Schär Chromosoma 121:1-20 (2012)). Such enzymes include modification-specific endonucleases or modification-specific glycosylases. Modified purines for use with modification-specific glycosylases include 3-methyladenine (3 mA) and 7-methylguanine (7mG). Modified pyrimidines for use with modification specific-glycosylases may include 5-methylcytosine (5mC), 5-formylcytosine (5fC), and 5-carboxycytosine (5caC). Selective removal of uracil and 8-oxoG using DNA repair enzymes are already used in certain sequencing platforms.

Because only one strand of the mosaic end, called the “transferred strand” is covalently appended to the library insert during transposition, incorporation of such a modified base, specifically into the mosaic end transferred strand, could enable selective cleavage and removal of the mosaic end transferred strand. However, this type of mosaic end cleavage and removal would require mutation of the mosaic end sequence from its canonical sequence (SEQ ID NO: 1).

TABLE 5
Examples of base modifications and enzymatic strategies for fBLT

| | Possible | Possible |
|---|---|---|
| Base | modification-specific | modification-specific |
| modification | N-glycosylases* | endonucleases |
| Uracil | UNG/UDG | |
| Inosine | | Endo V |
| Ribose base | | RNAse HII |
| 8-oxoguanine | | Fpg, OGG |
| Thymine glycol | EndoIII (Nth), Endo VIII | |
| Modified | hAAG | |
| purines (e.g., | | |
| 3 mA and 7 mG) | | |
| Modified | TDG, MBD4 | ROS1 |
| pyrimidines | | |
| (e.g., mC, fC, | | |
| caC) | | |

*N-glycosylases can be paired with an AP lyase/endonuclease (e.g., EndoIII or EndoVIII). As an alternative, abasic sites are chemically labile and may be cleaved with heat and/or basic conditions.

In Table 5, Endo=endonuclease, FPG=formamidopyrimidine-DNA glycosylase, OGG=oxoguanine glycosylase (OGG), hAAG=Human 3-alkyladenine DNA glycosylase, UNG=uracil-N-glycosylase, Nth=cloned nth gene, TDG=thymine-DNA glycosylase, MBD4=mammalian DNA glycosylase-methyl-CpG binding domain protein 4, and ROS1=endonuclease ROS1 (with bifunctional DNA glycosylase/lyase activity).

Disclosed herein is a modified transposon end sequence comprising a mosaic end sequence, wherein the mosaic end sequence comprises one or more mutation as compared to a wild-type mosaic end sequence, wherein the mutation comprises a substitution with a uracil; an inosine; a ribose; an 8-oxoguanine; a thymine glycol; a modified purine (such as 3 mA or 7 mG); or a modified pyrimidine. In some embodiments, these substitutions are used in methods to cleave the transposon end after transposition, as described below.

In some embodiments, the mosaic end sequence may be a mosaic end sequence for use with a Tn5 transposase. In some embodiments, a modified transposon end sequence has mutations in a mosaic end sequence as compared to SEQ ID NO: 1.

In some embodiments, a modified transposon end sequence comprises a mosaic end sequence comprising one or more mutation as compared to SEQ ID No: 1, wherein the one or more mutations comprise a substitution at A16, C17, A18, and/or G19. In some embodiments, a modified transposon end sequence comprises a mosaic end sequence comprising a substitution at A16. In some embodiments, a modified transposon end sequence comprises a mosaic end sequence comprising a substitution at C17. In some embodiments, a modified transposon end sequence comprises a mosaic end sequence comprising a substitution at A18. In some embodiments, a modified transposon end sequence comprises a mosaic end sequence comprising a substitution at G19. In some embodiments, the modified transposon end sequence comprises SEQ ID NOs: 5, 7, 9, or 16-24.

In some embodiments, the mosaic end sequence comprises more than one mutation. In some embodiments, the mosaic end sequence comprises no more than 8 mutations as compared to the wild-type sequence (in some embodiment SEQ ID NO: 1).

Additional mutations may also be present in a mosaic end sequence, in addition to the one or more mutations at A16, C17, A18, and/or G19. In some embodiments, the mosaic end sequence comprises one or more mutations as compared to SEQ ID NO: 1 in addition to the one or more mutations at A16, C17, A18, and/or G19. In some embodiments, the mosaic end sequence comprises from one to four substitution mutations as compared to SEQ ID NO: 1 in addition to the one or more mutations at A16, C17, A18, and/or G19.

In some embodiments, the mosaic end sequence has one substitution mutation as compared to SEQ ID NO: 1 in addition to the one or more mutations at A16, C17, A18, and/or G19. In some embodiments, the mosaic end sequence has two substitution mutations as compared to SEQ ID NO: 1 in addition to the one or more mutations at A16, C17, A18, and/or G19. In some embodiments, the mosaic end sequence has three substitution mutations as compared to SEQ ID NO: 1 in addition to the one or more mutations at A16, C17, A18, and/or G19. In some embodiments, the mosaic end sequence has four substitution mutations as compared to SEQ ID NO: 1 in addition to the one or more mutations at A16, C17, A18, and/or G19.

In some embodiments, the substitution at A16 is A16T, A16C, A16G, A16U, A16Inosine, A16Ribose, A16-8-oxoguanine, A16Thymine glycol, A16Modified purine, or A16Modified pyrimidine; the substitution at C17 is C17T, C17A, C17G, C17U, C17Inosine, C17Ribose, C17-8-oxoguanine, C17Thymine glycol, C17Modified purine, or C17Modified pyrimidine; the substitution at A18 is A18G, A18T, A18C, A18U, A18Inosine, A18Ribose, A18-8-oxoguanine, A18Thymine glycol, A18Modified purine, or A18Modified pyrimidine; and/or the substitution at G19 is G19T, G19C, G19A, G19U, G19Inosine, G19Ribose, G19-8-oxoguanine, G19Thymine glycol, G19Modified purine, or G19Modified pyrimidine. In some embodiments, the modified purine is 3 mA or 7 mG. In some embodiments, the modified pyrimidine is 5-methylcytosine, 5-formylcytosine, or 5-carboxycytosine.

In some embodiments, the mutation comprises a substitution with a uracil; an inosine; a ribose; an 8-oxoguanine; a thymine glycol; a modified purine; and/or a modified pyrimidine. In some embodiments, these mutations allow for methods to cleave the mosaic end sequence after transposition.

In some embodiments, the modified transposon end sequence comprises a mutation at A16, C17, A18, or G19.

In some embodiments, the modified transposon end sequence comprises two mutations chosen from mutations at A16, C17, A18, or G19. In some embodiments, the modified transposon end sequence comprises three mutations chosen from mutations at A16, C17, A18, or G19. In some embodiments, the modified transposon end sequence comprises four mutations at A16, C17, A18, and G19.

In some embodiments, the modified transposon end sequence has from one to four substitution mutations as compared to SEQ ID NO: 1 at A16, C17, A18, and/or G19. In some embodiments, the modified transposon end sequence has one substitution mutation as compared to the wild-type sequence (in some embodiments SEQ ID NO: 1). In some embodiments, the modified transposon end sequence has two substitution mutations as compared to the wild-type sequence (in some embodiments SEQ ID NO: 1). In some embodiments, the modified transposon end sequence has three substitution mutations as compared to the wild-type sequence (in some embodiments SEQ ID NO: 1). In some embodiments, the modified transposon end sequence has four substitution mutations as compared to the wild-type sequence (in some embodiments SEQ ID NO: 1).

D. Immobilized Transposomes

In some methods and compositions presented herein, transposome complexes are immobilized to the solid support. In some embodiments, the transposome complexes are immobilized to the support via one or more polynucleotides, such as a polynucleotide comprising a transposon end sequence. In some embodiments, the transposome complex may be immobilized via a linker molecule coupling the transposase enzyme to the solid support. In some embodiments, both the transposase enzyme and the polynucleotide are immobilized to the solid support. When referring to immobilization of molecules (e.g. nucleic acids) to a solid support, the terms “immobilized” and “attached” are used interchangeably herein and both terms are intended to encompass direct or indirect, covalent or non-covalent attachment, unless indicated otherwise, either explicitly or by context. In some embodiments, covalent attachment may be used, but generally all that is required is that the molecules (e.g. nucleic acids) remain immobilized or attached to the support under the conditions in which it is intended to use the support, for example in applications requiring nucleic acid amplification and/or sequencing.

In some embodiments, the transposome complex composition comprises or consists of at least one transposon with one or more other nucleotide sequences in addition to the transposon sequences. Such nucleotide sequences may be referred to as polynucleotides.

In some embodiments, the transposome complexes comprise a transposase bound to a first polynucleotide comprising a 3′ portion comprising a transposon end sequence and a first tag.

In some embodiments, the transposome complexes comprise a transposase bound to a first polynucleotide comprising a 3′ portion comprising a transposon end sequence and a second tag.

Thus, in some embodiments, the transposon composition comprises a transferred strand with one or more other nucleotide sequences 5′ of the transferred transposon sequence, e.g., a tag sequence or an adapter sequence. In some embodiments, in addition to the transferred transposon sequence, the transposon comprises one or more other tag portions or tag domains. In some embodiments, in addition to the transferred transposon sequence, the transposon comprises one or more adapters.

In some embodiments, the transposome complex is immobilized to the solid support via the first polynucleotide.

In some embodiments, the transposome complexes comprise a second polynucleotide comprising a region complementary to the transposon end sequence. In some embodiments, the transposome complex is immobilized to the solid support via the second polynucleotide.

In some embodiments, the lengths of the double-stranded fragments in the immobilized library are adjusted by increasing or decreasing the density of transposome complexes on the solid support.

Means of immobilizing transposome complexes have been described in U.S. Ser. No. 10/920,219, which is incorporated herein in its entirety. In some embodiments, the first transposon comprises an affinity element. In some embodiments, the affinity element is attached to the 5′ end of the first transposon. In some embodiments, the first transposon comprises a linker. In some embodiments, the linker has a first end attached to the 5′ end of the first transposon and a second end attached to an affinity element.

In some embodiments, the transposome complex comprises a second transposon complementary to at least a portion of the first transposon end sequence. In some embodiments, the second transposon comprises an affinity element. In some embodiments, the affinity element is attached to the 3′ end of the second transposon. In some embodiments, the second transposon comprises a linker. In some embodiments, the linker has a first end attached to the 3′ end of the second transposon and a second end attached to an affinity element.

In some embodiments, the affinity element is biotin or dual biotin. In some embodiments, a solid support is coated with streptavidin.

In some embodiments, the solid support is a bead, and the methods use DNA BLTs (bead-linked transposomes). Transposomes bound to a surface (e.g., BLTs) can tagment long molecules of double-stranded DNA and make template libraries on beads or other surfaces (U.S. Pat. No. 9,683,230). Anchoring transposomes to beads gives novel properties such as controllable insert size and yield. This is the basis of the Illumina DNA Flex PCR-Free technology, previously known as Illumina's Nextera technology.

As transposon ends have affinity for DNA, DNA BLTs do not require a capture oligonucleotide for immobilization on beads, and instead DNA can be immobilized using polynucleotides comprising a transposon end sequence. Alternatively, a capture oligonucleotide may be used to capture DNA molecules.

Representative products employing immobilized transposomes (i.e., bead-linked transposomes) include Illumina® DNA Prep, (S) Tagmentation and Illumina® RNA Prep, (L) Tagmentation or Illumina® RNA Prep, (L) Tagmentation.

1. Solid Supports for Immobilized Transposomes

Certain embodiments may make use of solid supports comprised of an inert substrate or matrix (e.g. glass slides, polymer beads, etc.) which has been functionalized, for example by application of a layer or coating of an intermediate material comprising reactive groups which permit covalent attachment to biomolecules, such as polynucleotides. Examples of such supports include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such embodiments, the biomolecules (e.g. polynucleotides) may be directly covalently attached to the intermediate material (e.g. the hydrogel) but the intermediate material may itself be non-covalently attached to the substrate or matrix (e.g. the glass substrate). The term “covalent attachment to a solid support” is to be interpreted accordingly as encompassing this type of arrangement.

The terms “solid surface,” “solid support” and other grammatical equivalents herein refer to any material that is appropriate for or can be modified to be appropriate for the attachment of the transposome complexes. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. Particularly useful solid supports and solid surfaces for some embodiments are located within a flow cell apparatus. Exemplary flow cells are set forth in further detail below.

In some embodiments, the solid support comprises a patterned surface suitable for immobilization of transposome complexes in an ordered pattern. A “patterned surface” refers to an arrangement of different regions in or on an exposed layer of a solid support. For example, one or more of the regions can be features where one or more transposome complexes are present. The features can be separated by interstitial regions where transposome complexes are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. In some embodiments, the transposome complexes are randomly distributed upon the solid support. In some embodiments, the transposome complexes are distributed on a patterned surface. Exemplary patterned surfaces that can be used in the methods and compositions set forth herein are described in U.S. application Ser. No. 13/661,524 or US Pat. App. Publ. No. 2012/0316086, each of which is incorporated herein by reference.

In some embodiments, the solid support comprises an array of wells or depressions in a surface. This may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The composition and geometry of the solid support can vary with its use. In some embodiments, the solid support is a planar structure such as a slide, chip, microchip and/or array. As such, the surface of a substrate can be in the form of a planar layer. In some embodiments, the solid support comprises one or more surfaces of a flow cell. The term “flow cell” as used herein refers to a chamber comprising a solid surface across which one or more fluid reagents can be flowed. Examples of flow cells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

In some embodiments, the solid support or its surface is non-planar, such as the inner or outer surface of a tube or vessel. In some embodiments, the solid support comprises microspheres or beads. By “microspheres” or “beads” or “particles” or grammatical equivalents herein is meant small discrete particles. Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and teflon, as well as any other materials outlined herein for solid supports may all be used. “Microsphere Selection Guide” from Bangs Laboratories, Fishers Ind. is a helpful guide. In certain embodiments, the microspheres are magnetic microspheres or beads.

The beads need not be spherical; irregular particles may be used. Alternatively or additionally, the beads may be porous. The bead sizes range from nanometers, i.e. 100 nm, to millimeters, i.e. 1 mm, with beads from 0.2 micron to 200 microns, or from 0.5 to 5 microns, although in some embodiments smaller or larger beads may be used.

The density of these surface bound transposomes can be modulated by varying the density of the first polynucleotide or by the amount of transposase added to the solid support. For example, in some embodiments, the transposome complexes are present on the solid support at a density of at least 10³, 10⁴, 10⁵, or 10⁶ complexes per mm².

When double stranded DNA is synthesized for tagmenting on transposomes on a solid support (such as a BLT), the transposome complexes will tagment generate ds fragments coupled at both ends to the surface. In some embodiments, the length of bridged fragments can be varied by changing the density of the transposome complexes on the surface. In certain embodiments, the length of the resulting bridged fragments is less than 100 bp, 200 bp, 300 bp, 400 bp, 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, 2400 bp, 2500 bp, 2600 bp, 2700 bp, 2800 bp, 2900 bp, 3000 bp, 3100 bp, 3200 bp, 3300 bp, 3400 bp, 3500 bp, 3600 bp, 3700 bp, 3800 bp, 3900 bp, 4000 bp, 4100 bp, 4200 bp, 4300 bp, 4400 bp, 4500 bp, 4600 bp, 4700 bp, 4800 bp, 4900 bp, 5000 bp, 10000 bp, 30000 bp or less than 100,000 bp. In such embodiments, the bridged fragments can then be amplified into clusters using standard cluster chemistry, as exemplified by the disclosure of U.S. Pat. Nos. 7,985,565 and 7,115,400, the contents of each of which is incorporated herein by reference in its entirety.

Attachment of a nucleic acid to a support, whether rigid or semi-rigid, can occur via covalent or non-covalent linkage(s). Exemplary linkages are set forth in U.S. Pat. Nos. 6,737,236; 7,259,258; 7,375,234 and 7,427,678; and US Pat. Pub. No. 2011/0059865 A1, each of which is incorporated herein by reference. In some embodiments, a nucleic acid or other reaction component can be attached to a gel or other semisolid support that is in turn attached or adhered to a solid-phase support. In such embodiments, the nucleic acid or other reaction component will be understood to be solid-phase.

In some embodiments, the solid support comprises microparticles, beads, a planar support, a patterned surface, or wells. In some embodiments, the planar support is an inner or outer surface of a tube.

In some embodiments, this solid support is for tagmenting DNA and is termed a DNA bead-linked transposome (DNA BLT).

In some embodiments, the solid support further comprises a transposase bound to the first polynucleotide to form a transposome complex.

In some embodiments, solid supports comprise a library of tagged fragments immobilized thereon prepared according to any of the methods described herein.

In some embodiments, a kit comprises a solid support as described herein. In some embodiments, a kit further comprises a transposase. In some embodiments, a kit further comprises a composition as described herein.

In some embodiments, the transposome complexes may be solution-phase transposome complexes, such as those described in U.S. Pat. No. 9,683,230, which is incorporated by reference herein in its entirety. In some embodiments, solution-phase transposome complexes are used to generate tagged fragments in solution. In some embodiments, a method further comprises contacting solution-phase transposome complexes with immobilized DNA fragments under conditions whereby the DNA fragments are further fragmented by the solution-phase transposome complexes; thereby obtaining immobilized nucleic acid fragments having one end in solution.

2. Exemplary BLTs

As used herein, a BLT may refer to any type of bead with transposome complexes immobilized on its surface. A range of BLTs are known in the art.

An exemplary BLT is Illumina RNA Prep with Enrichment (L) product (See RNA Prep with Enrichment (L) Tagmentation Reference Guide, Document #1000000124435 v02, Illumina, 2020 (“Document 1000000124435”)). Due to their ability to produce fragments with larger inserts, such BLTs are often currently preferred for methods of preparation of RNA libraries (i.e., libraries of cDNA fragments generated from RNA samples).

Another exemplary BLT is Illumina® DNA Prep, (S) Tagmentation. In some embodiments, such BLTs produce fragments with smaller inserts based on the higher density of transposomes on the beads, as compared to other BLTs.

In some embodiments, BLTs are used in 1-pot library preparations (with combined cDNA and library preparation). In some embodiments, BLTs with higher transposome activity improve library yield in 1-pot library preparations (such as with a higher density of transposomes on the bead). One skilled in the art could use standard experimentation to determine the best conditions for using a given BLT in the present methods.

E. Adapters and Tags

In some embodiments, the first transposon comprises one or more adapter sequences. In some embodiments, a first transposon comprises a 3′ transposon end sequence and a 5′ adaptor sequence. In some embodiments, the 5′ adaptor sequence is a tag sequence. Fragmentation mediated by transposome complexes comprising a first transposon comprising a 3′ transposon end sequence and a 5′ tag can be used in methods to generate a library of tagged fragments.

In some embodiments, the tag is an adapter sequence. In some embodiments, the adaptor sequence comprises a primer sequence, an index tag sequence, a capture sequence, a barcode sequence, a cleavage sequence, or a sequencing-related sequence, or a combination thereof. As used herein, a sequencing-related sequence may be any sequence related to a later sequencing step. A sequencing-related sequence may work to simplify downstream sequencing steps. For example, a sequencing-related sequence may be a sequence that would otherwise be incorporated via a step of ligating an adaptor to nucleic acid fragments. In some embodiments, the adaptor sequence comprises a P5 or P7 sequence (or their complement) to facilitate binding to a flow cell in certain sequencing methods. This disclosure is not limited to the type of adaptor sequences which could be used and a skilled artisan will recognize additional sequences which may be of use for library preparation and next generation sequencing.

The terms “tag” as used herein refers to a portion or domain of a polynucleotide that exhibits a sequence for a desired intended purpose or application. Tag domains can comprise any sequence provided for any desired purpose. For example, in some embodiments, a tag domain comprises one or more restriction endonuclease recognition sites. In some embodiments, a tag domain comprises one or more regions suitable for hybridization with a primer for a cluster amplification reaction. In some embodiments, a tag domain comprises one or more regions suitable for hybridization with a primer for a sequencing reaction. It will be appreciated that any other suitable feature can be incorporated into a tag domain. In some embodiments, the tag domain comprises a sequence having a length from 5 bp to 200 bp. In some embodiments, the tag domain comprises a sequence having a length from 10 bp to 100 bp. In some embodiments, the tag domain comprises a sequence having a length from 20 bp to 50 bp. In some embodiments, the tag domain comprises a sequence having a length of 5, 6, 7, 8, 9, 10, 20, 30, 40, 50, 60, 70, 80, 90, 100, 150 or 200 bp.

The tag can include one or more functional sequences or components (e.g., primer sequences, anchor sequences, universal sequences, spacer regions, or index tag sequences) as needed or desired.

In some embodiments, the tag comprises a region for cluster amplification. In some embodiments, the tag comprises a region for priming a sequencing reaction.

In some embodiments, the method further comprises amplifying the fragments on the solid support by reacting a polymerase and an amplification primer corresponding to a portion of the first transposon. In some embodiments, a portion of the first transposon comprises an amplification primer. In some embodiments, the tag of the first transposon comprises an amplification primer.

In some embodiments a tag comprises an A14 primer sequence (SEQ ID NO: 11). In some embodiments, a tag comprises a B15 primer sequence (SEQ ID NO: 12).

In some embodiments, transposomes on an individual bead carry a unique index, and if a multitude of such indexed beads are employed, phased transcripts will result.

III. Methods of cDNA Preparation

A difficulty in RNA sequencing (such as RNA-Seq from Illumina) analysis is the need to convert RNA into DNA prior to library preparation, adding significant time and complexity to the procedure. However, RNA is an important molecule, providing quantitative functional information about transcriptomes and metatranscriptomes. Importantly, in many cases for disease surveillance, RNA is of paramount interest as the most pathogenic families of viruses have RNA genomes (See “Select Agents and Toxins List,” CDC/USDA (2020) or www.selectagents.gov). Easier methods for RNA library preparation presented herein may be preferred by users, especially those who may be less familiar with NGS protocols. Further, the present methods may allow simple, cost-effective automation to process multiple samples. FIGS. 4-10 show data from present mesophilic methods of cDNA preparation in a 1-step process (i.e., present method). FIGS. 11-12B show data from present thermostable methods of cDNA preparation in a 1-step process.

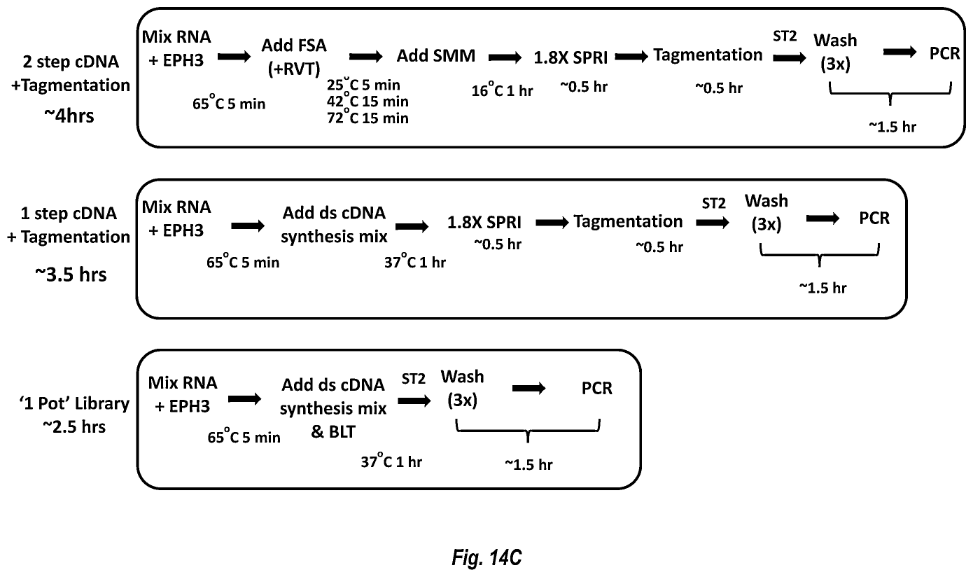

Methods described herein can convert RNA from a sample into double-stranded cDNA in as little as 15 minutes. This is a significant reduction compared to 110 minutes in conventional protocols (such as Illumina Stranded Total RNA Prep or Illumina Stranded mRNA Prep, as shown in FIG. 1). Additionally, the number of touchpoints (i.e., actions by the user) are reduced, making the protocol easier for end users. A more detailed overview of the different in timeline for a standard 2-step method of cDNA preparation versus the present 1-step method is shown in FIG. 14B.

Described herein is a method of preparing double-stranded cDNA comprising (i) combining primers with a sample comprising RNA and allowing binding of the primers to an RNA and (ii) combining the sample with a composition for ds-cDNA preparation described herein and preparing double-stranded cDNA by an isothermal reaction.

FIG. 3 presents a model of the method. In some embodiments, a composition with a balance of the individual enzymatic components aids the preparation of ds-cDNA. In some embodiments, the generation of the first strand cDNA outpaces the rate at which RNA is nicked by RNAse H. In some embodiments, the activity of reverse transcriptase exceeds the activity of RNAse H.

In some embodiments, the sample comprises 10 ng or more of RNA. In some embodiments, the sample comprises less than 10 ng of RNA.

In some embodiments, the reverse transcriptase produces a first strand of cDNA. In some embodiments, the reverse transcriptase produces a DNA:RNA duplex comprising the first strand of cDNA and a strand of RNA. In some embodiments, the RNAse H nicks the RNA strand in the DNA:RNA duplex to produce RNA fragments. In some embodiments, the DNA polymerase extends a second strand of DNA by priming from the RNA fragments. In some embodiments, the RNA nickase and/or the 5′-3′ and 3′ to 5′ activity of the DNA polymerase removes the RNA fragments and RNA overhangs.

In some embodiments, the rate of producing the first strand of cDNA by the reverse transcriptase is greater than the rate of nicking of the RNA by the RNA nickase. In some embodiments, the activity of the reverse transcriptase exceeds the activity of the RNA nickase. In this way, a first strand of cDNA is produced before the RNA is degraded by the RNA nickase.

In some embodiments, the DNA polymerase has 5′-3′ exonuclease activity and/or 3′-5′ exonuclease activity, wherein this activity produces blunt-ended double-stranded cDNA. In some embodiments, a DNA polymerase has strand displacement activity.

In some embodiments, the dNTPs are used by both the reverse transcriptase and the DNA polymerase.

In some embodiments, depletion of unwanted RNA is performed before preparing cDNA. In some embodiments, this unwanted RNA is ribosomal RNA (rRNA). In some embodiments, the method comprises performing off-target RNA depletion with the sample comprising RNA before combining primers with the sample comprising RNA.

In some embodiments, enrichment of desired RNA is performed before preparing cDNA. In some embodiments, the desired RNA is mRNA.

A. Primers

A variety of different primers may be used in this method.

In some embodiments, the primers comprise random primers (also known as randomer primers). In some embodiments, the randomer primers do not target to specific sequences (i.e., the primers are non-targeted). In some embodiments, the randomer primers allow unbiased production of ds-cDNA from the RNA, as the user is not selecting specific sequences for the randomer primers to bind. In some embodiments, a method with randomer primers avoids biased or non-uniform preparation of cDNA, as the method does not comprise primers that are designed to bind to specific sequences within the RNA. In some embodiments, no gene-specific or transcript-specific primer is required for the method. Instead, random primers can be used to prime the synthesis of the first strand of cDNA to start the coordinated process of transforming RNA into double-stranded cDNA, as described herein. In some embodiments, random primers avoid the need for specialized primers, such as AT-rich primers, that may be needed to promote binding of targeted primers.

In some embodiments, the primers bind specifically to one or more sequences comprised in the RNA. Such primers that specifically bind to one or more sequences comprised in the RNA may be termed “targeted primers.” In some embodiments, targeted primers allow production of ds-cDNA from specific regions of the RNA. This targeted production of ds-cDNA is based on the fact that a first strand of cDNA will only be generated based in the region where the targeted primers bind.

In some embodiments, the primers comprise hexamer primers. In some embodiments, the primers comprise random hexamer primers.

In some embodiments, the primers comprise a mixture comprising randomer primers and targeted primers.

In some embodiments, the primers comprise primers comprising chemically modified nucleotides. In some embodiments, the primers comprising chemically modified nucleotides render the RNA bound by the primers resistant to cleavage by the RNA nickase. In some embodiments, the RNA nickase is RNAse H, and the RNA bound by the primers comprising chemically modified nucleotides is resistant to cleavage by RNAse H.

In some embodiments, the chemically modified nucleotides comprise methylphosphonate residues.

B. Isothermal Reaction

An advantage of the present method, as compared to methods known in the art, is the ability to produce ds-cDNA from RNA without requiring temperature changes. As shown in FIG. 1, prior art methods require up to 5 temperature changes. These temperature changes require more sophisticated equipment (such as a programmable thermocycler) and user input, as compared to the present isothermal method.

In some embodiments, an isothermal reaction to prepare ds-cDNA may be performed with mesophilic or thermostable compositions, as described above.

In some embodiments, the methods do not require temperature changes. In some embodiments, the method does not require computer-controlled temperature modulation in a thermal cycler. In some embodiments, the reaction temperature does not need to be changed after a composition described herein is added. In some embodiments, primer binding may be performed at a different temperature, and the reaction temperature is changed when the composition is added, but the reaction does not require a temperature change when preparing ds-cDNA from the RNA bound by the primers.

In some embodiments, the isothermal reaction is at a temperature of from 30° C.-49° C. In some embodiments, the isothermal reaction is at a temperature of 37° C. In some embodiments, an isothermal reaction at a temperature of from 30° C.-49° C. is performed using a composition for mesophilic ds-cDNA preparation, as described herein.

In some embodiments, the isothermal reaction is at a temperature of from 50° C.-72° C. In some embodiments, the isothermal reaction is at a temperature of 50° C. In some embodiments, an isothermal reaction at a temperature of from 50° C.-72° C. is performed using a composition for thermostable ds-cDNA preparation, as described herein.

In some embodiments, the RNA exhibits a secondary structure that normally inhibits first strand synthesis at temperature below 50° C. In some embodiments, an isothermal reaction performed at 50° C.-72° C. shows improved ds-cDNA yield or coverage compared to an isothermal reaction performed at a temperature below 50° C. Secondary structures of RNA, such as hairpins, would be well-known to those skilled in the art.

C. Incubation Time

An advantage of the present method may be a reduced reaction time as compared to methods known in the art. As shown in FIG. 1, prior art methods require up to 110 minutes, while the present method can be performed in 15 minutes or less.

In some embodiments, the isothermal reaction is incubated for 60 minutes or less, 45 minutes or less, 30 minutes or less, 20 minutes or less, 15 minutes of less, or 10 minutes or less. In some embodiments, the isothermal reaction is incubated for 15 minutes or less. In some embodiments, the isothermal reaction is incubated for 10 minutes or less.

In some embodiments, incubations of at least 10 minutes, at least 20 minutes, at least 30 minutes, at least 45 minutes, or at least 60 minutes yield ds-cDNA for library preparation.

D. rRNA Depletion or mRNA Enrichment

In some embodiments, desired RNA is enriched or unwanted RNA is depleted before beginning cDNA preparation (e.g., before binding primers to RNA). In this way, cDNA is only produced from desired RNA. Such methods can avoid reagent waste and unnecessary analysis of data.

In some embodiments, a method comprises removing unwanted RNA before preparing cDNA. In this way, cDNA is not made from the unwanted RNA. In some embodiments, the unwanted RNA is abundant RNA that would otherwise lead to generation of significant amounts of cDNA related to the unwanted RNA.

In some embodiments, the removing of unwanted RNA is by enzymatic depletion. In some embodiments, the remaining RNA after depletion of unwanted RNA is then converted into cDNA by the methods described herein.

In some embodiments, the unwanted RNA comprises ribosomal RNA or beta globin transcripts. A number of different types of rRNA depletion methods are known, including those disclosed in U.S. Pat. No. 9,745,570 and WO 2020132304, each of which is incorporated by reference in its entirety herein.

In some embodiments, the rRNA is cytoplasmic or mitochondrial. In some embodiments, the rRNA is human, mouse, rat, gram (−) bacterial, or gram (+) bacterial rRNA. In some embodiments, the unwanted RNA comprises beta globin transcripts. In some embodiments, the unwanted RNA is human beta globin transcripts.

In some embodiments, rRNA depletion can be performed with an Illumina® Ribo-Zero Plus rRNA Depletion kit or other similar kit or method.

In some embodiments, a method comprises enriching desired RNA before preparing cDNA. In some embodiments, the desired RNA is mRNA.

In some embodiments, mRNA enrichment comprises amplification with a poly-T primer or binding of mRNA to capture beads. In some embodiments, capture beads comprise a surface with capture oligonucleotides comprising poly-T sequences.

### IV. Methods of Library Preparation

In some embodiments, methods allow for preparation of a library of double-stranded DNA fragments in a single reaction vessel from a sample comprising RNA. In some embodiments, the double-stranded DNA fragments comprise double-stranded cDNA prepared as described above. In some embodiments, a method of library preparation includes a method of preparing double-stranded cDNA from RNA, as described herein. In some embodiments, combined cDNA and library preparation can be performed in a single reaction vessel, which may be referred to herein as a “1-pot” method. In some embodiments, a 1-pot method of cDNA and library preparation comprises a 1-step method of cDNA preparation together with preparation of library fragments in the same reaction vessel. In some embodiments, preparation of library fragments in a 1-pot method is by tagmentation using BLTs.

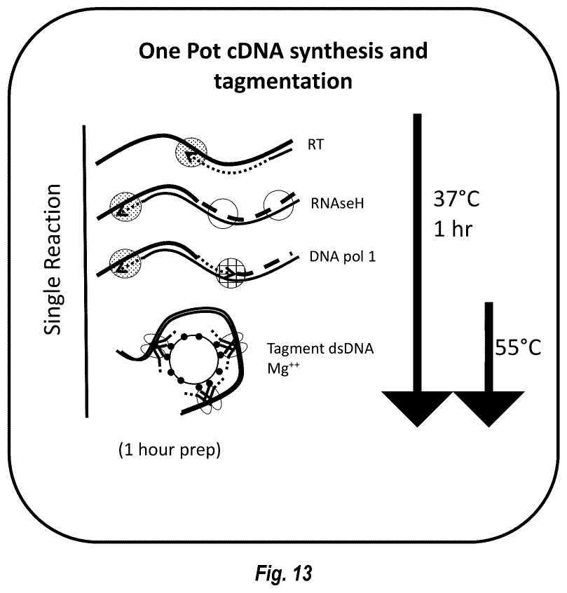

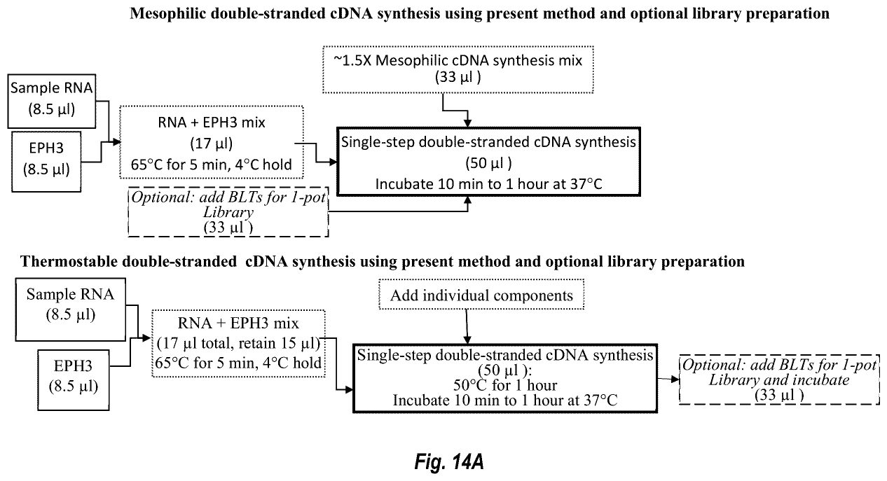

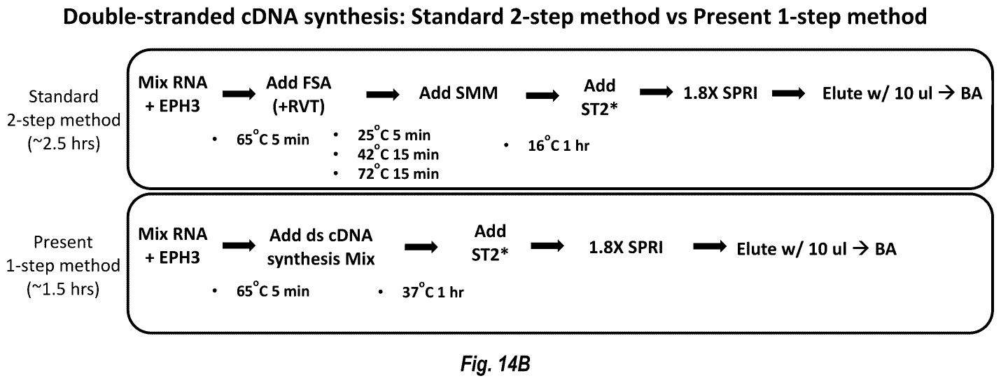

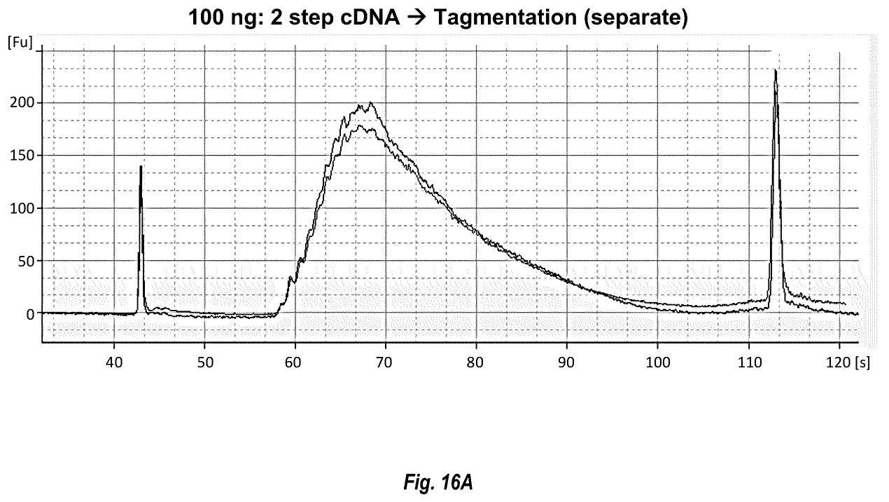

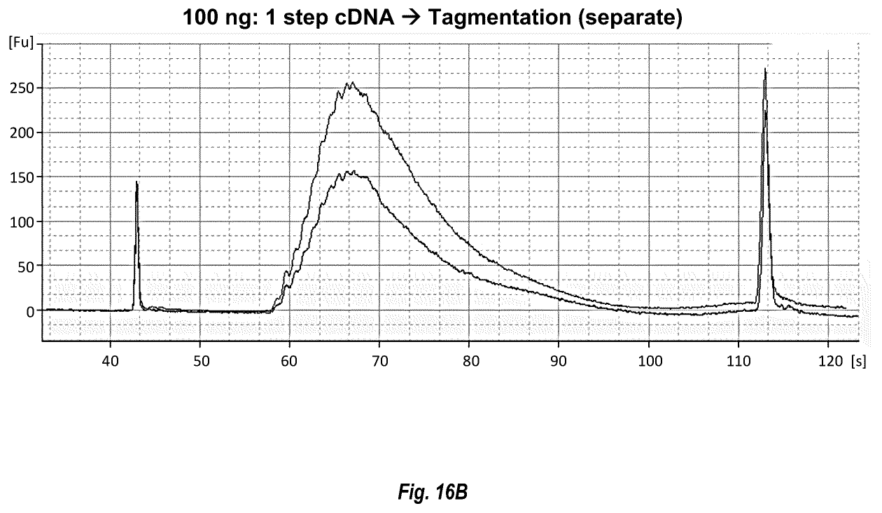

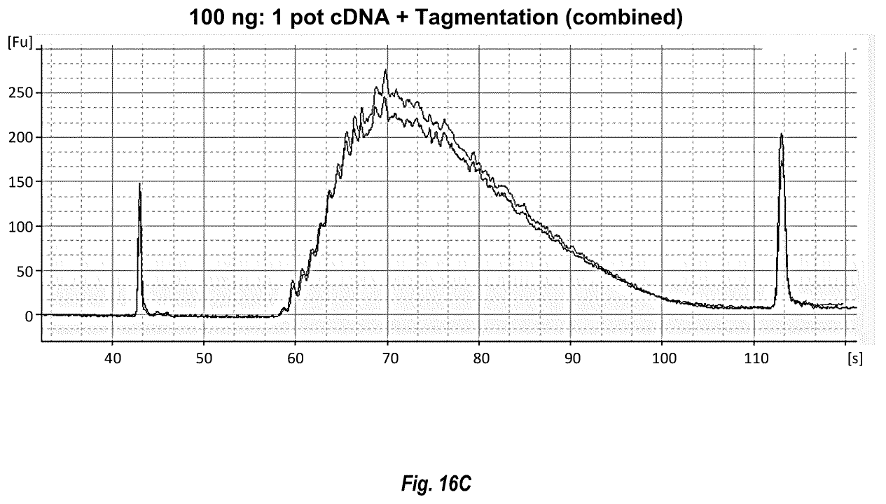

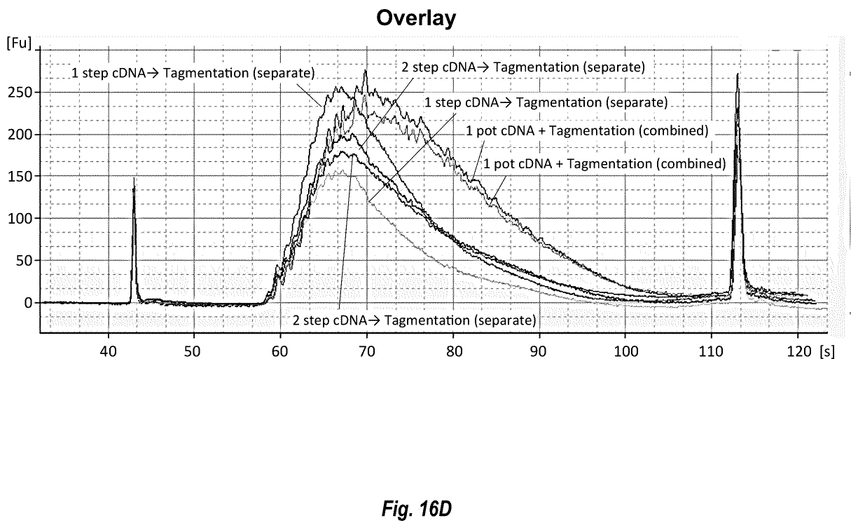

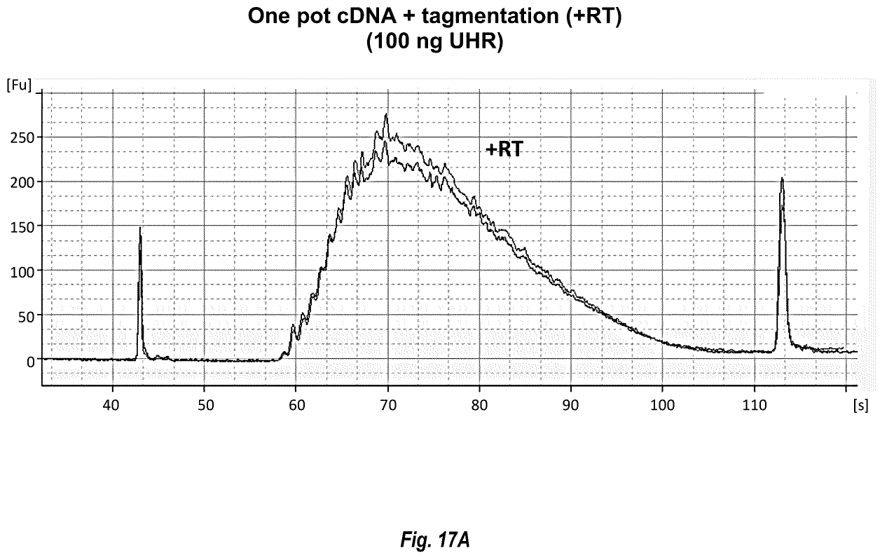

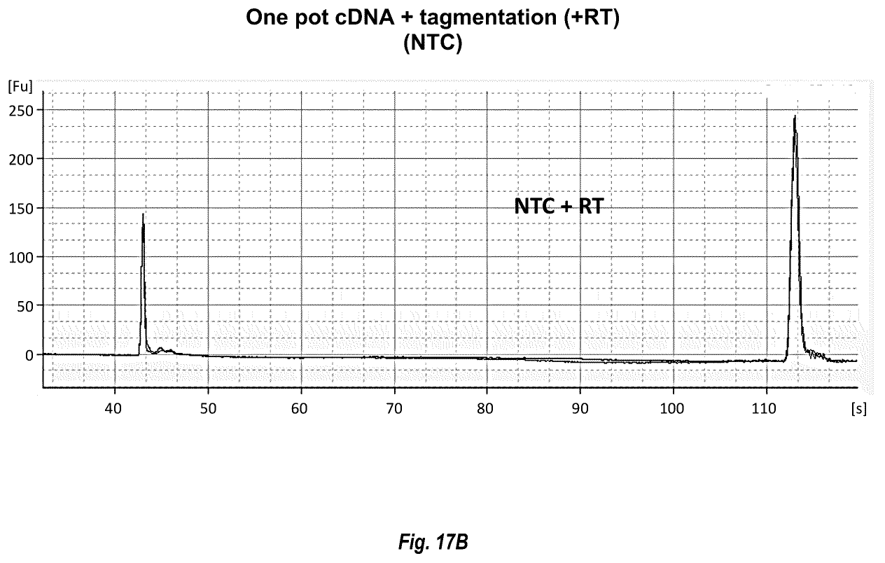

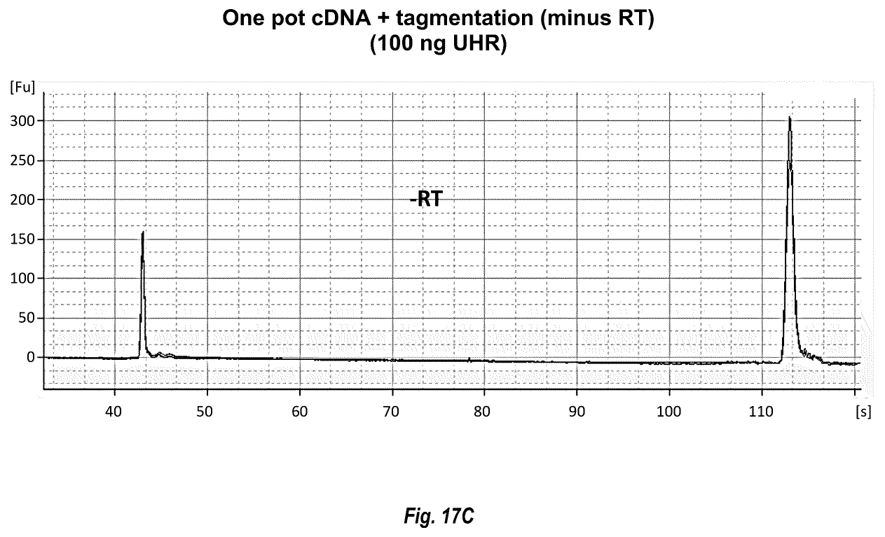

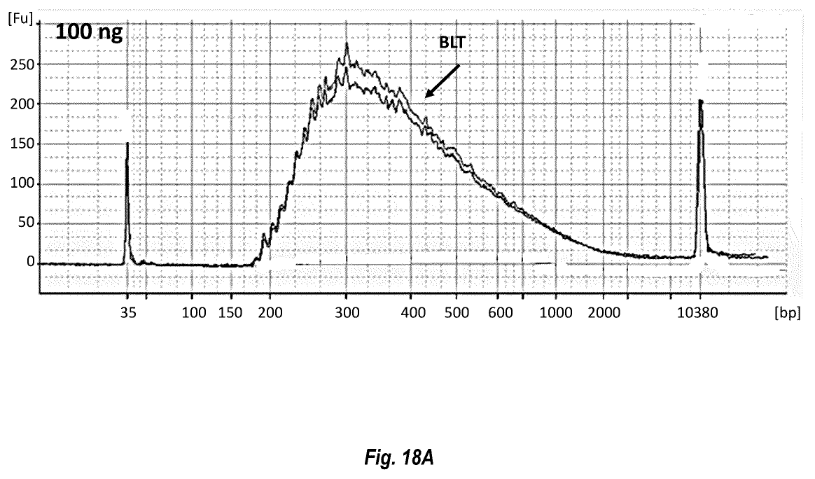

A representative overview of a 1-pot method of library preparation with combined cDNA and library preparation is shown in FIG. 13. As shown in FIGS. 14A and 14B, library preparation with BLTs can be included in methods also comprising mesophilic or thermostable double-stranded cDNA synthesis. In this way, a user can generate a library for sequencing from RNA in a single reaction vessel. The advantages of time with a 1-pot library preparation are shown in FIG. 14C, wherein a 1-pot library preparation can save 1-1.5 hours of time over other preparation methods, as well as avoid multiple hands-on steps for the user. FIG. 16C shows that comparable fragments are generated with a 1-pot (combined cDNA preparation and tagmentation) as compared to a 2-step cDNA preparation (FIG. 16A) or a 1-step cDNA preparation (FIG. 16B) followed by separate tagmentation, as summarized in FIG. 16D. FIGS. 17A-18B and 20A-24 provide additional data on 1-pot tagmentation libraries and comparative data with other library preparations.

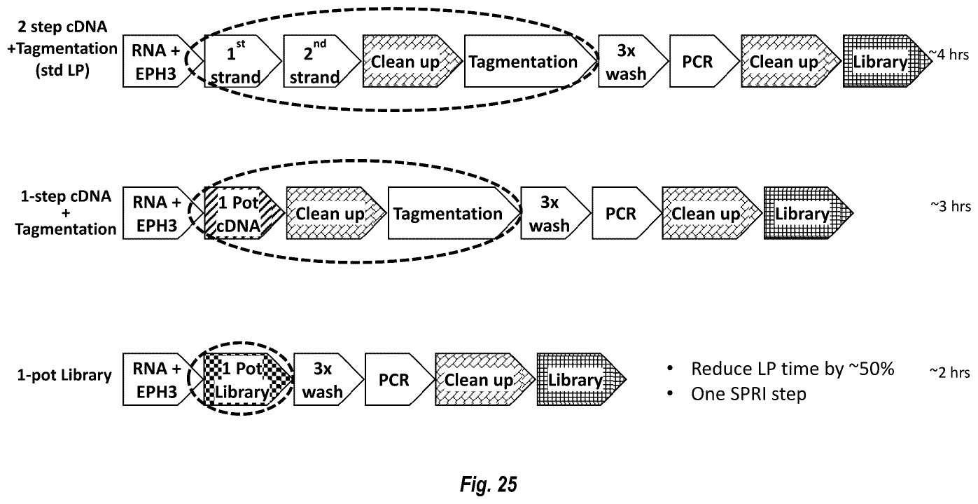

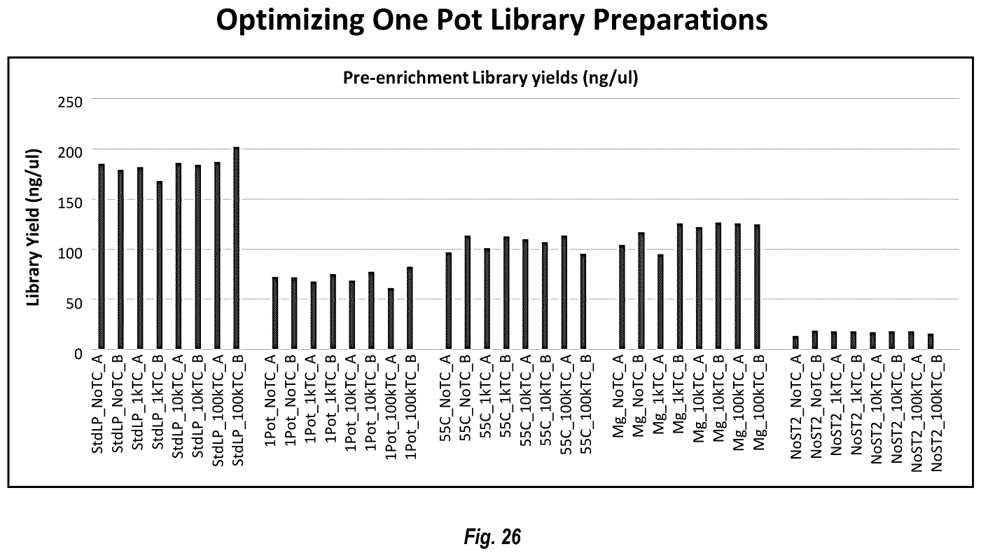

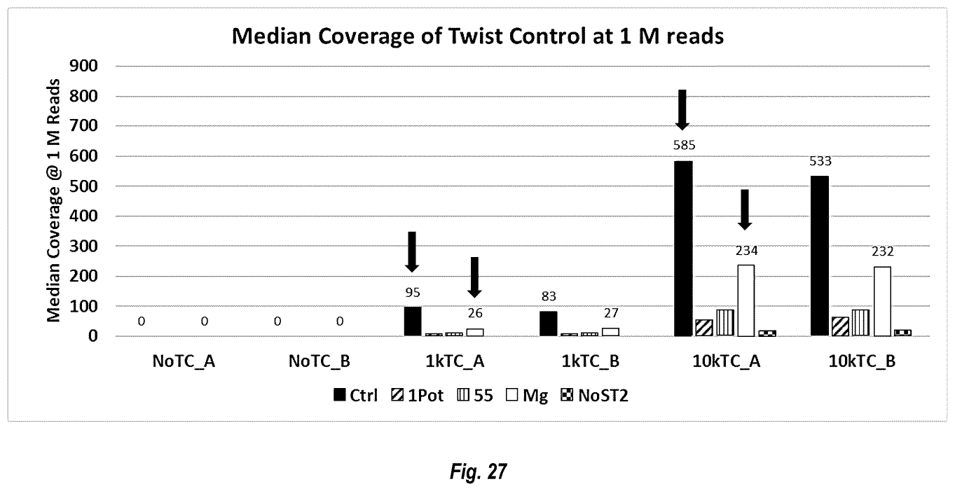

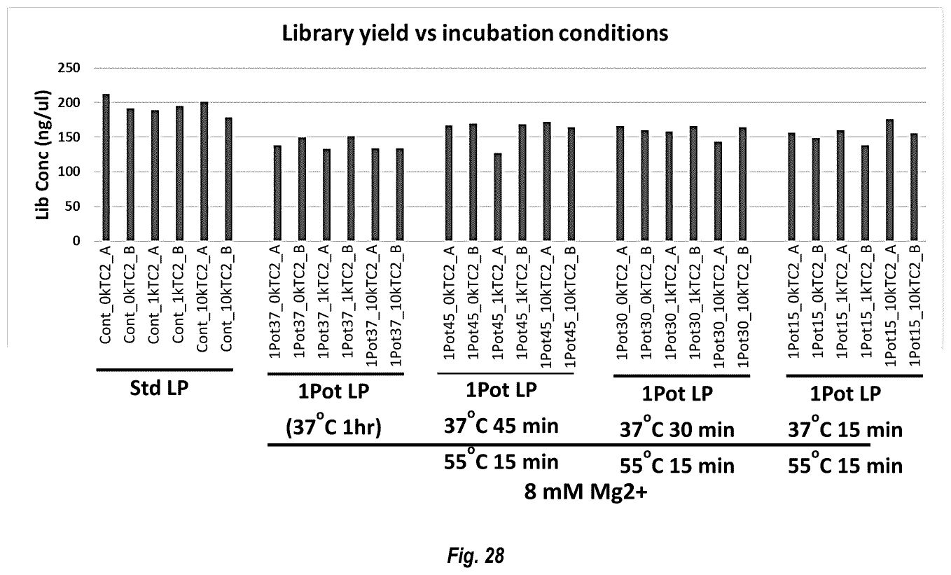

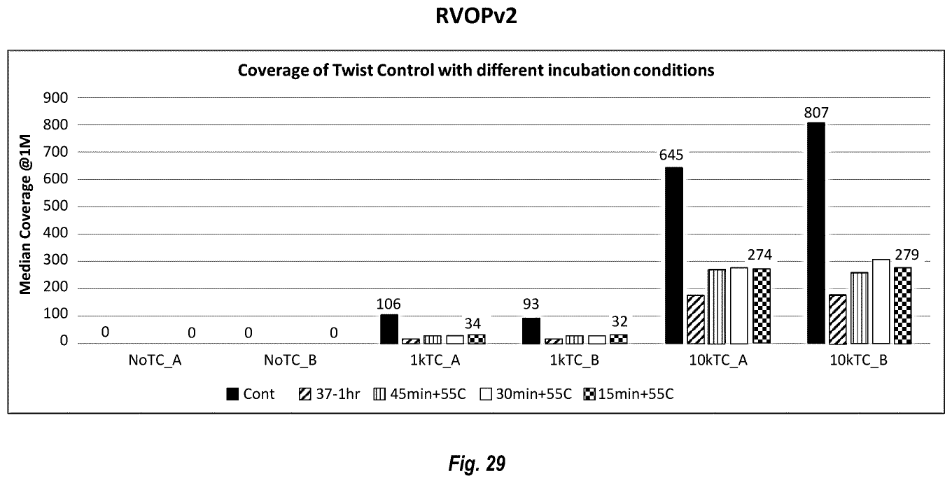

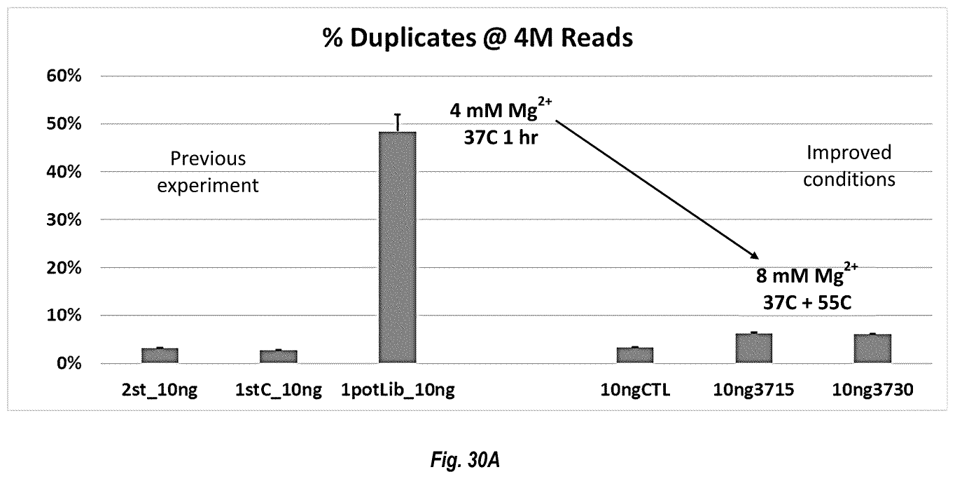

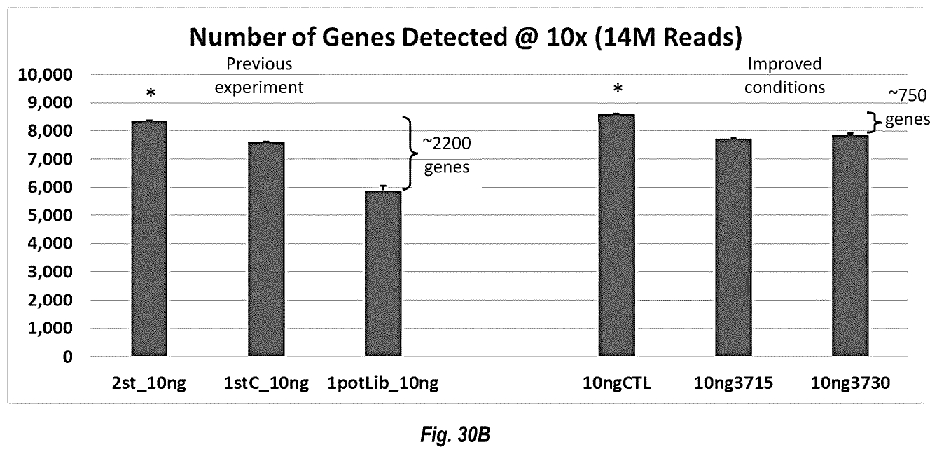

FIG. 25 outlines advantages of the 1-pot library preparation, with a reduction in time to approximately 2 hours from 4 hours with 2-step cDNA preparation followed by separate tagmentation or 3 hours with 1-step cDNA preparation followed by separate tagmentation. In some embodiments, the 1-pot library preparation method only requires a single clean-up step (such as with SPRI beads). In some embodiments, SPRI or other cleanup is not performed between preparing double-stranded cDNA by an isothermal reaction and preparing double-stranded cDNA fragments. Thus, the 1-pot library preparation can save overall time and hands-on time for the user.

The number of steps required to transform a target nucleic acid such as DNA into adaptor-modified templates ready for next generation sequencing can be minimized by tagmentation. Tagmentation results in the simultaneous fragmentation of the target nucleic acid and ligation of the adaptors to the 5′ ends of both strands of duplex nucleic acid fragments. Where the transposome complexes are support-bound (i.e., immobilized to a solid support), the resulting fragments are bound to the solid support following the tagmentation reaction (either directly in the case of the 5′ linked transposome complexes, or via hybridization in the case of the 3′ linked transposome complexes).

In some embodiments, tagmentation is performed after preparation of double-stranded cDNA. In some embodiments, the tagmentation is performed on bead-linked transposomes (BLTs). BLTs will not bind RNA itself, and thus BLTs in the reaction will fragment double-stranded cDNA prepared from the RNA. In some embodiments, methods using BLTs results in a library of cDNA fragments that are immobilized to a bead.

In some embodiments, reactions for preparing cDNA from RNA and for preparing fragments of the cDNA are run simultaneously. In some embodiments, the preparation of library fragments can occur as the cDNA is being prepared, without requiring a change in reaction vessel. In some embodiments, the cDNA may be fragmented as it is being prepared, without purification of the cDNA before the fragmenting. In some embodiments, all steps of a library preparation are performed in a single reaction vessel.

In some embodiments, a method of preparing a library of double-stranded cDNA fragments comprises combining primers with a sample comprising RNA and allowing binding of the primers to an RNA; and combining the sample with a composition described herein and (i) preparing double-stranded cDNA by an isothermal reaction and (ii) preparing double-stranded cDNA fragments.

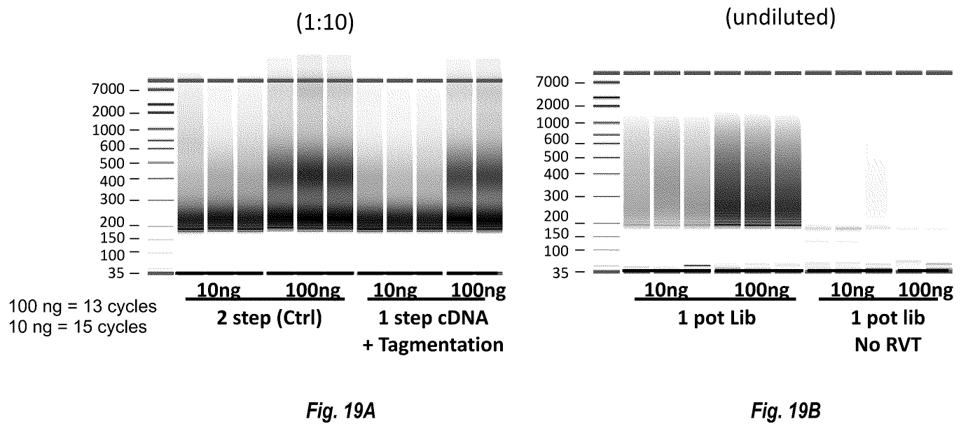

In some embodiments, unwanted RNA is depleted or desired RNA is enriched before library preparation. In some embodiments, the unwanted RNA is rRNA and the desired RNA is mRNA. Representative data with rRNA depletion with 1-pot library preparation are shown in FIGS. 19A-19B.

In some embodiments, the combining primers with a sample and the combining the sample with a composition are performed in the same step.

In some embodiments, (i) preparing double-stranded cDNA and (ii) preparing double-stranded cDNA fragments are both performed by a single isothermal reaction.

In some embodiments, (i) preparing double-stranded cDNA and (ii) preparing double-stranded cDNA fragments are performed at different temperatures.

In some embodiments, the (i) preparing double-stranded cDNA and (ii) preparing double-stranded cDNA fragments are performed in a single reaction vessel.

In some embodiments, the combining primers with a sample comprising RNA comprises mixing the sample comprising RNA with an elution, primer, and fragmentation mix. An exemplary elution, primer, and fragmentation mix would be EPH3 (Illumina®).

In some embodiments, the combining primers with a sample comprising RNA is performed at 55° C. or higher. In some embodiments, the combining primers with a sample comprising RNA is performed at 65° C.

In some embodiments, fragments of a cDNA library are prepared by tagmentation. In some embodiments, the temperature of the reaction is increased after double-stranded cDNA preparation to increase efficiency of tagmentation. In some embodiments, RNA is converted into double-stranded cDNA and then into a library of double-stranded DNA fragments in a single reaction vessel, wherein double-stranded cDNA is prepared via an isothermal reaction and the temperature of the reaction vessel is increased to improve efficiency of tagmentation to prepare fragments. In some embodiments, RNA is converted into double-stranded cDNA and then into a library of double-stranded DNA fragments in a single reaction vessel via a single isothermal reaction.

In some embodiments, the isothermal reaction for preparing double-stranded cDNA is at a temperature of from 30° C.-49° C. In some embodiments, the isothermal reaction for preparing double-stranded cDNA is at a temperature of 37° C. or above. In some embodiments, the isothermal reaction for preparing double-stranded cDNA is at a temperature of 37° C. In some embodiments, the isothermal reaction for preparing double-stranded cDNA is at a temperature of 55° C.

In some embodiments, (i) preparing double-stranded cDNA and (ii) preparing double-stranded cDNA fragments are both performed by a single isothermal reaction at 37° C. In some embodiments, the preparing double-stranded cDNA fragments and/or preparing double-stranded cDNA fragments are performed above 37° C. In some embodiments, preparing double-stranded cDNA fragments is performed at 55° C. In some embodiments, preparing double-stranded cDNA is performed at 37° C. and preparing double-stranded cDNA fragments is performed at 55° C.

In some embodiments, the Mg²⁺ concentration of the composition used for the method is 1 mM to 50 mM, optionally wherein the Mg²⁺ concentration is 5 mM to 20 mM, further optionally wherein the Mg²⁺ concentration is 8 mM. In some embodiments, (1) the reverse transcriptase, the RNA nickase, and/or the DNA polymerase are thermostable enzymes and (2) the Mg²⁺ concentration of the composition used for the method is 1 mM to 50 mM, optionally wherein the Mg²⁺ concentration is 5 mM to 20 mM, further optionally wherein the Mg²⁺ concentration is 8 mM.

In some embodiments, the rate of producing the first strand of cDNA by the reverse transcriptase is greater than the rate of nicking of the RNA by the RNA nickase. In some embodiments, the activity of the reverse transcriptase exceeds the activity of the RNA nickase. In some embodiments, (i) preparing double-stranded cDNA by an isothermal reaction and (ii) preparing double-stranded cDNA fragments are performed with a total incubation of 60 minutes or less or 30 minutes or less.

In some embodiments, gap-fill ligation is performed after preparation of double-stranded DNA fragments. In some embodiments, double-stranded DNA fragments are amplified.

In some embodiments, double-stranded DNA fragments are sequenced. In some embodiments, tagmentation incorporates adapters for sequencing double-stranded DNA fragments. In some embodiments, double-stranded DNA fragments are amplified before sequencing. In some embodiments, double-stranded DNA fragments are not amplified before sequencing.

In some embodiments, the present methods allow for faster and simpler preparation of a library for sequencing from a starting sample comprising RNA. For example, the present methods can allow for enhanced pathogen surveillance of RNA viruses, although sequencing from any type of RNA sample can be enhanced with the present methods.

In some embodiments, depletion of unwanted RNA or enrichment of desired RNA may be performed before library preparation, as described above.

In some embodiments, a stop tagmentation buffer is added after preparing double-stranded cDNA fragments. In some embodiments, the prepared double-stranded cDNA fragments are purified. In some embodiments, the prepared double-stranded cDNA fragments are sequenced. In some embodiments, the prepared double-stranded cDNA fragments are purified and then sequenced.

In some embodiments, methods of library preparation comprise steps to fragment modified transposon ends. In some embodiments, methods of library preparation comprise steps to incorporate UMIs. In some embodiments, methods of library preparation further comprise steps such as gap-fill ligation, amplification, and/or sequencing of fragments.

In some embodiments, the method of library preparation includes targeted enrichment. In some embodiments, the step of preparing double-stranded cDNA fragments includes enrichment for target fragments. In some embodiments, amplification of double-stranded cDNA fragments is performed with target-specific primers that bind to and allow for amplification of target fragments.

A. Optimization of Tagmentation Reaction

In some embodiments, a method of 1-Pot library preparation comprises optimizations to increase the yield of the tagmentation reaction. Results of such optimizations are shown in FIGS. 26-30B.

In some embodiments, a step of incubation above 37° C. is included in a 1-Pot library preparation. It is known in the art that tagmentation preferentially occurs above 37° C., such as at 55° C. In some embodiments, a 1-Pot library method includes an incubation of the reaction at 55° C. In some embodiments, the incubation at 55° C. is 15 minutes or 30 minutes. In some embodiments, the incubation at 55° C. occurs after an incubation at 37° C. (i.e., the reaction temperature is increased after allowing for double-stranded cDNA preparation at 37° C.).

In some embodiments, the entire 1-Pot library preparation is performed by an isothermal reaction at greater than 37° C. In some embodiments, the entire 1-Pot library preparation is performed by an isothermal reaction at 40° C. or greater, 45° C. or greater, 50° C. or greater, or 55° C. or greater. In some embodiments, the entire 1-Pot library preparation is performed at 55° C. Such a library preparation at 55° C. can be performed using thermostable enzymes described herein.

In some embodiments, a 1-Pot library preparation is performed with a relatively high Mg²⁺ concentration. Transposases, such as Tn5, are known to use magnesium ions as a cofactor in tagmentation reactions. Experiments in FIGS. 26-30B showed that increasing the Mg²⁺ helped to improve library yield of 1-Pot library preparations. In some embodiments, the Mg²⁺ concentration is 1 mM to 50 mM. In some embodiments, the Mg²⁺ concentration is 5 mM to 20 mM. In some embodiments, the Mg²⁺ concentration is 8 mM. In some embodiments, a user empirically determines the Mg²⁺ concentration that produces an optimum yield. In some embodiments, the Mg²⁺ concentration is optimized to increase the tagmentation reaction while minimizing potential for RNA degradation.

B. Transposition Reactions for Fragmenting

Transposition is an enzyme-mediated process by which DNA sequences are inserted, deleted, and duplicated within genomes. This process has been adapted for broad uses in fragmented double-stranded nucleic acids (such as double-stranded DNA and DNA:RNA duplexes). Transposition can generate DNA fragments without using the standard fragmentase protocols.

The well-studied E. coli Tn5 transposon mobilizes by a “cut-and-paste” transposition mechanism. First, the Tn5 transposase Tnp (hereafter, referred to as Tn5) recognizes conserved substrate sequences on either side of transposon DNA, which is then excised, or “cut” from the genome. Tn5 then inserts, or “pastes” this transposon DNA into a target DNA.

Tn5 has been leveraged in many library preparation reagents (such as those of Illumina) for its ability to “tagment,” that is, simultaneously “tag” and “fragment” genomic DNA, thus greatly decreasing the time and complexity involved in conventional sonication/ligation-based library preparation protocols. In order to support its use with library preparation, Tn5 is pre-loaded with transposons consisting of the conserved substrate sequence, called a “mosaic end” or “end sequence” appended to adapter sequences (e.g., Illumina's A14 and B15 adapter sequences). Then, this transposome complex, comprising the Tn5 transposase and the adapter-bearing transposon sequence, is mixed with a genomic DNA sample. Resulting library preparation transposons bear only short adapter sequences, thus simultaneously leading to fragmentation of the genomic DNA and tagging with the short adapter sequences.

In some embodiments, transposition with the modified transposon ends described herein gives comparable results as transposition with a wild-type (i.e., transposon end not comprising a mutation described herein). In some embodiments, preparing fragments with a transposome complex described herein leads to preparation of at least 50%, at least 60%, at least 70%, at least 80%, or at least 90% the number of fragments, as compared with preparing fragments with a transposome complex that comprises a first transposon comprising a transposon end sequence comprising a wildtype mosaic end sequence comprising SEQ ID No: 1.

1. Mosaic End Removal

In some embodiments, selective cleavage of a mosaic end using enzymes is a highly attractive mechanism for transforming Tn5 into a fragmentase system (i.e., to generate fragments lacking mosaic ends). As used herein, a “base modification” or “DNA base modification” refers to the position of a modified base (such as those described in Table 3) in a double-stranded nucleic acid that will be recognized by an enzyme (such as (a) an endonuclease or (b) a combination of a DNA glycosylase and heat, basic conditions, or an endonuclease/lyase that recognizes abasic sites), triggering cleavage at this modified base. In some embodiments, an endonuclease or DNA glycosylase is modification-specific.

In some embodiments, a base modification is cleaved using (1) an endonuclease or (2) a combination of a DNA glycosylase and heat, basic conditions, or an endonuclease/lyase that recognizes abasic sites. For example, a DNA glycosylase may produce an abasic site that is then acted upon by heat, basic conditions, or an endonuclease/lyase that recognizes abasic sites. USER reagents are an exemplary enzyme mix comprising a DNA glycosylase and an endonuclease/lyase that recognizes abasic sites. The user may choose how to cleave at an abasic site depending on their preferred workflow. A modification-specific endonuclease can cleave a modified base in a 1-step reaction or a modification-specific glycosylase followed by an AP lyase/endonuclease or heat can cleave a modified base in a 2-step reaction.

Fragments prepared from such a transposition reaction followed by cleavage at a modified base will comprise inserts with 5′ overhangs with 5′ phosphate and 3′-OH, and 0-3 bases of ME sequence, depending on the site of modification at one or more of positions 16-19 of SEQ ID NO: 1.

In some embodiments, cleavage of the modified mosaic end sequence is mediated by (a) an endonuclease or (b) a combination of a DNA glycosylase and heat, basic conditions, or an endonuclease/lyase that recognizes abasic sites. In some embodiments, (a) an endonuclease or (b) a combination of a DNA glycosylase and heat, basic conditions, or an endonuclease/lyase that recognizes abasic sites can mediate cleavage at a uracil, an inosine, a ribose, an 8-oxoguanine, a thymine glycol, a modified purine, and/or a modified pyrimidine.

In some embodiments, the (a) an endonuclease or (b) a combination of a DNA glycosylase and heat, basic conditions, or an endonuclease/lyase that recognizes abasic sites is a USER, endonuclease V, RNAse HII, formamidopyrimidine-DNA glycosylase (FPG), oxoguanine glycosylase (OGG), endonuclease III (Nth), endonuclease VIII, a mixture of human alkyl adenine DNA glycosylase plus endonuclease VIII or endonuclease III, a mixture of and either thymine-DNA glycosylase (TDG) or mammalian DNA glycosylase-methyl-CpG binding domain protein 4 (MBD4) plus endonuclease VIII or endonuclease III, or DNA glycosylase/lyase ROS1 (ROS1). In some embodiments, ROS1 can function as a modification-endonuclease based on its bifunctional glycosylase/lyase activity.

In some embodiments, the modified transposon end sequence comprises a uracil and the mixture is a N-glycosylase and an apurinic or apyrimidinic site (AP) lyase/endonuclease is a uracil-specific excision reagent (USER). In some embodiments, the USER is a mixture of uracil DNA glycosylase and endonuclease VIII or endonuclease III.

In some embodiments, the modified transposon end sequence comprises an inosine and the endonuclease is endonuclease V. In some embodiments, the modified transposon end sequence comprises a ribose and the endonuclease is RNAse HII.

In some embodiments, the modified transposon end sequence comprises a 8-oxoguanine and the endonuclease is formamidopyrimidine-DNA glycosylase (FPG) or oxoguanine glycosylase (OGG).

In some embodiments, the modified transposon end sequence comprises a thymine glycol and the DNA endonuclease is endonuclease III (Nth) or endonuclease VIII.

In some embodiments, the modified transposon end sequence comprises a modified purine and the DNA glycosylase and endonuclease/lyase that recognizes abasic sites is a mixture of human alkyl adenine DNA glycosylase (hAAG) plus endonuclease VIII or endonuclease III.

In some embodiments, the modified transposon end sequence comprises a modified pyrimidine and the DNA glycosylase is TDG or MBD4 and the endonuclease/lyase that recognizes abasic sites is endonuclease VIII or endonuclease III. An alternative modification-specific endonuclease for use with a modified transposon end comprising a modified pyrimidine is ROS1.

In some embodiments, a first transposon comprises a modified transposon end sequence comprising more than one mutation chosen from a uracil, an inosine, a ribose, an 8-oxoguanine, a thymine glycol, a modified purine, and/or a modified pyrimidine and the endonuclease or DNA glycosylase and endonuclease/lyase that recognizes abasic sites are comprised in a mixture. In some embodiments, the endonuclease or DNA glycosylase and endonuclease/lyase that recognizes abasic sites comprises more than enzyme chosen from a USER, endonuclease V, RNAse HII, formamidopyrimidine-DNA glycosylase (FPG), oxoguanine glycosylase (OGG), endonuclease III (Nth), endonuclease VIII, a mixture of hAAG plus endonuclease VIII/endonuclease III, or a mixture of TDG or MBD4 together with endonuclease VIII/endonuclease III, or ROS1. In some embodiments, methods with modified transposon end sequences comprising more than one mutation and an endonuclease and/or a combination of DNA glycosylase and endonuclease/lyase that recognizes abasic sites improves the efficiency of cleavage of the mosaic end sequence as compared to methods with a modified transposon end sequences comprising a single mutation and a single endonuclease or combination of DNA glycosylase and endonuclease/lyase that recognizes abasic sites. For ROS1, a single endonuclease has both glycosylase and lyase function.

In some embodiments, a method of fragmenting a double-stranded nucleic acid comprises combining a sample comprising double-stranded nucleic acid with a transposome complex and preparing fragments.

In some embodiments, a method of preparing double-stranded nucleic acid fragments that lack all or part of the first transposon end comprises combining a sample comprising nucleic acid with transposome complexes and preparing fragments; and combining the sample with (1) an endonuclease or (2) a combination of a DNA glycosylase and heat, basic conditions, or an endonuclease/lyase that recognizes abasic sites and cleaving the first transposon end at the uracil, inosine, ribose, 8-oxoguanine, thymine glycol, a modified purine, and/or a modified pyrimidine within the mosaic sequence to remove all or part of the first transposon end from the fragments. In some embodiments, the modified purine is 3-methyladenine or 7-methylguanine. In some embodiments, the modified pyrimidine is 5-methylcytosine, 5-formylcytosine, or 5-carboxycytosine. In some embodiments, this method cleaves all or part of the first transposon end (the transferred strand) from the fragments.

In some embodiments, cleaving the first transposon end generates a sticky end for ligating an adapter. As used herein, a “sticky end” is an end of a double-stranded fragment wherein one strand is longer than the other (i.e., there is an overhang) and the overhang allows for ligation of an adapter comprising a complementary overhang.

In some embodiments, adapters are added after removing all or part of the first transposon end from fragments. In some embodiments, adapters are added by ligation. In some embodiments, end repair and A-tailing mixes enable ligation of adapters. One skilled in the art would be aware of other means to add adapters, such as PCR amplification or Click chemistry.

2. Ligation of Adapters

In some embodiments, a method of preparing double-stranded nucleic acid fragments comprising adapters comprises combining a sample comprising nucleic acid with the transposome complexes described herein and preparing fragments; combining the sample with (1) an endonuclease or (2) a combination of a DNA glycosylase and heat, basic conditions, or an endonuclease/lyase that recognizes abasic sites and cleaving the first transposon end at the uracil, inosine, ribose, 8-oxoguanine, thymine glycol, modified purine, and/or a modified pyrimidine within the mosaic end sequence to remove all or part of the first transposon end from the fragments; and ligating an adapter onto the 5′ and/or 3′ ends of the fragments.

In some embodiments, adapters comprising sequence sequences are ligated onto library fragments after removal of all or part of the mosaic end sequence. Fragments that been subjected to ligation of an adapter to the 5′ and/or 3′ end of the fragment may be termed “tagged fragments.”

In some embodiments, the ligating is performed with a DNA ligase.

In some embodiments, the adapter comprises a double-stranded adapter.

In some embodiments, adapters are added to the 5′ and 3′ end of fragments. In some embodiments, the adapters added to the 5′ and 3′ end of the fragments are different.

A wide variety of library preparation methods comprising a step of adapter ligation are known in the art, such as TruSeq and TruSight Oncology 500 (See, for example, TruSeq® RNA Sample Preparation v2 Guide, 15026495 Rev. F, Illumina, 2014). Adapters used with other ligation methods may be used in the present method (See, for example, Illumina Adapter Sequences, Illumina, 2021). Adapters for use in the present invention also include those described in WO 2008/093098, WO 2008/096146, WO 2018/208699, and WO 2019/055715, which are each incorporated by reference in their entirety herein.

In some embodiments, adapter ligation may allow for more flexible incorporation of adapters (such as adapters with longer lengths) as compared to methods of tagging fragments via tagmentation (wherein adapter sequences are incorporated into fragments during the transposition reaction). In some methods involving tagmentation, additional adapter sequences may be incorporated by PCR reactions (such as those described in US Patent Publication No. 20180201992A1), and the present methods may obviate the need for an additional PCR step to incorporate additional adapter sequences.

Ligation technology is commonly used to prepare NGS libraries for sequencing. In some embodiments, the ligation step uses an enzyme to connect specialized adapters to both ends of DNA fragments. In some embodiments, an A-base is added to blunt ends of each strand, preparing them for ligation to the sequencing adapters. In some embodiments, each adapter contains a T-base overhang, providing a complementary overhang for ligating the adapter to the A-tailed fragmented DNA.

Adapter ligation protocols are known to have advantages over other methods. For example, adapter ligation can be used to generate the full complement of sequencing primer hybridization sites for single, paired-end, and indexed reads. In some embodiments, adapter ligation eliminates a need for additional PCR steps to add the index tag and index primer sites.

In some embodiments, the adapter comprises a unique molecular identifier (UMI), primer sequence, anchor sequence, universal sequence, spacer region, index sequence, capture sequence, barcode sequence, cleavage sequence, sequencing-related sequence, and combinations thereof. As used herein, a “barcode sequence” refers to a sequence that may be used to differentiate samples. As used herein, a sequencing-related sequence may be any sequence related to a later sequencing step. A sequencing-related sequence may work to simplify downstream sequencing steps. For example, a sequencing-related sequence may be a sequence that would otherwise be incorporated via a step of ligating an adapter to nucleic acid fragments. In some embodiments, the adapter sequence comprises a P5 or P7 sequence (or their complement) to facilitate binding to a flow cell in certain sequencing methods.

In some embodiments, the adapter comprises a UMI. In some embodiments, an adapter comprising a UMI is ligated to both the 3′ and 5′ end of fragments.

In some embodiments, the adapter may be a forked adapter. As used herein, a “forked adapter” refers to an adapter comprising two strands of nucleic acid, wherein the two strands each comprise a region that is complementary to the other strand and a region that is not complementary to the other strand. In some embodiments, the two strands of nucleic acid in the forked adapter are annealed together before ligation, with the annealing based on complementary regions. In some embodiments, the complementary regions each comprise 12 nucleotides. In some embodiments, a forked adapter is ligated to both strands at the end of a double-stranded DNA fragment. In some embodiments, a forked adapter is ligated to one end of a double-stranded DNA fragment. In some embodiments, a forked adapter is ligated to both ends of a double-stranded DNA fragment. In some embodiments, the forked adapters on opposite ends of a fragment are different. In some embodiments, one strand of the forked adapter is phosphorylated at its 5′ end to promote ligation to fragments. In some embodiments, one strand of the forked adapter has a phosphorothioate bond directly before a 3′ T. In some embodiments, the 3′ T is an overhang (i.e., not paired with a nucleotide in the other strand of the forked adapter). In some embodiments, the 3′ T overhang can basepair with an A-tail present on a library fragment. In some embodiments, the phosphorothioate bond blocks exonuclease digestion of the 3′ T overhang. In some embodiments, PCR with partially complementary primers is used after adapter ligation to extend ends and resolve the forks.

In some embodiments, an adapter may comprise a tag. The terms “tag” as used herein refers to a portion or domain of a polynucleotide that exhibits a sequence for a desired intended purpose or application. Tag domains can comprise any sequence provided for any desired purpose. For example, in some embodiments, a tag domain comprises one or more restriction endonuclease recognition sites. In some embodiments, a tag domain comprises one or more regions suitable for hybridization with a primer for a cluster amplification reaction. In some embodiments, a tag domain comprises one or more regions suitable for hybridization with a primer for a sequencing reaction. It will be appreciated that any other suitable feature can be incorporated into a tag domain. In some embodiments, the tag domain comprises a sequence having a length from 5 bp to 200 bp. In some embodiments, the tag domain comprises a sequence having a length from 10 bp to 100 bp. In some embodiments, the tag domain comprises a sequence having a length from 20 bp to 50 bp. In some embodiments, the tag domain comprises a sequence having a length of 5, 6, 7, 8, 9, 10, 20, 30, 40, 50, 60, 70, 80, 90, 100, 150 or 200 bp.

The tag can include one or more functional sequences or components (e.g., primer sequences, anchor sequences, universal sequences, spacer regions, or index tag sequences) as needed or desired.

In some embodiments, the tag comprises a region for cluster amplification. In some embodiments, the tag comprises a region for priming a sequencing reaction.

In some embodiments, the method further comprises amplifying the fragments on the solid support by reacting a polymerase and an amplification primer corresponding to a portion of a tag. In some embodiments, a portion of the adapter ligated onto fragments after removal of all or part of the mosaic end sequence comprises an amplification primer. In some embodiments, the tag of the first transposon comprises an amplification primer.

In some embodiments a tag comprises an A14 primer sequence. In some embodiments, a tag comprises a B15 primer sequence.

In some embodiments, transposomes on an individual bead carry a unique index, and if a multitude of such indexed beads are employed, phased transcripts will result.

Adapters that are ligated onto library fragments can have advantages over adapters that are incorporated during tagmentation. For example, unique molecular identifiers (UMIs) can be used to enable high-sensitivity variant detection by labeling single fragments with unique sequence tags prior to PCR (See Jesse J. Salk, et al., Nature Reviews Genetics 19(5): 269-85 (2018)). Some library preparation products, such as TSO 500 (Illumina), include a ligation-based UMI offering in which the UMI sequence is incorporated adjacent to the library insert, enabling simultaneous sequencing as a part of the insert read. Therefore, development of fBLTs enables existing ligation-based products to be leveraged (such as use of existing adapters and protocols), while simultaneously enabling compatibility with existing enrichment workflows and onboard sequencing primers.

C. Gap-Fill Ligation

In some embodiments, gaps in the DNA sequence left after the transposition event can also be filled in using a strand displacement extension reaction, such one comprising a Bst DNA polymerase and dNTP mix. In some embodiments, a gap-fill ligation is performed using an extension-ligation mix buffer.

The library of double-stranded DNA fragments can then optionally be amplified (such as with cluster amplification) and sequenced with a sequencing primer.

D. Amplification

The present disclosure further relates to amplification of the DNA fragments (i.e., cDNA fragments) produced according to the methods provided herein. In some embodiments, immobilized DNA fragments produced by surface bound transposome mediated tagmentation can be amplified according to any suitable amplification methodology known in the art. In some embodiments, the immobilized DNA fragments are amplified on a solid support. In some embodiments, the solid support is the same solid support upon which the surface bound tagmentation occurs. In such embodiments, the methods and compositions provided herein allow sample preparation to proceed on the same solid support from the initial sample introduction step through amplification and optionally through a sequencing step.

For example, in some embodiments, the immobilized DNA fragments are amplified using cluster amplification methodologies as exemplified by the disclosures of U.S. Pat. Nos. 7,985,565 and 7,115,400, the contents of each of which is incorporated herein by reference in its entirety. The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules. Each cluster or colony on such an array is formed from a plurality of identical immobilized polynucleotide strands and a plurality of identical immobilized complementary polynucleotide strands. The arrays so-formed are generally referred to herein as “clustered arrays”. The products of solid-phase amplification reactions such as those described in U.S. Pat. Nos. 7,985,565 and 7,115,400 are so-called “bridged” structures formed by annealing of pairs of immobilized polynucleotide strands and immobilized complementary strands, both strands being immobilized on the solid support at the 5′ end, in some embodiments via a covalent attachment. Cluster amplification methodologies are examples of methods wherein an immobilized nucleic acid template is used to produce immobilized amplicons. Other suitable methodologies can also be used to produce immobilized amplicons from immobilized DNA fragments produced according to the methods provided herein. For example, one or more clusters or colonies can be formed via solid-phase PCR whether one or both primers of each pair of amplification primers are immobilized.

In other embodiments, DNA fragments are amplified in solution. For example, in some embodiments, DNA fragments are cleaved or otherwise liberated from a solid support and amplification primers are then hybridized in solution to the liberated molecules. In other embodiments, amplification primers are hybridized to immobilized DNA fragments for one or more initial amplification steps, followed by subsequent amplification steps in solution. Thus, in some embodiments an immobilized nucleic acid template can be used to produce solution-phase amplicons.

It will be appreciated that any of the amplification methodologies described herein or generally known in the art can be utilized with universal or target-specific primers to amplify immobilized DNA fragments. Suitable methods for amplification include, but are not limited to, the polymerase chain reaction (PCR), strand displacement amplification (SDA), transcription mediated amplification (TMA) and nucleic acid sequence based amplification (NASBA), as described in U.S. Pat. No. 8,003,354, which is incorporated herein by reference in its entirety. The above amplification methods can be employed to amplify one or more nucleic acids of interest. For example, PCR, including multiplex PCR, SDA, TMA, NASBA and the like can be utilized to amplify immobilized DNA fragments. In some embodiments, primers directed specifically to the nucleic acid of interest are included in the amplification reaction.

Other suitable methods for amplification of nucleic acids can include oligonucleotide extension and ligation, rolling circle amplification (RCA) (Lizardi et al., Nat. Genet. 19:225-232 (1998), which is incorporated herein by reference) and oligonucleotide ligation assay (OLA) (See generally U.S. Pat. Nos. 7,582,420, 5,185,243, 5,679,524 and 5,573,907; EP 0 320 308 B1; EP 0 336 731 B1; EP 0 439 182 B1; WO 90/01069; WO 89/12696; and WO 89/09835, all of which are incorporated by reference) technologies. It will be appreciated that these amplification methodologies can be designed to amplify immobilized DNA fragments. For example, in some embodiments, the amplification method can include ligation probe amplification or oligonucleotide ligation assay (OLA) reactions that contain primers directed specifically to the nucleic acid of interest. In some embodiments, the amplification method can include a primer extension-ligation reaction that contains primers directed specifically to the nucleic acid of interest. As a non-limiting example of primer extension and ligation primers that can be specifically designed to amplify a nucleic acid of interest, the amplification can include primers used for the GoldenGate assay (Illumina, Inc., San Diego, CA) as exemplified by U.S. Pat. Nos. 7,582,420 and 7,611,869, each of which is incorporated herein by reference in its entirety.

Exemplary isothermal amplification methods that can be used in a method of the present disclosure include, but are not limited to, Multiple Displacement Amplification (MDA) as exemplified by, for example Dean et al., Proc. Natl. Acad. Sci. USA 99:5261-66 (2002) or isothermal strand displacement nucleic acid amplification exemplified by, for example U.S. Pat. No. 6,214,587, each of which is incorporated herein by reference in its entirety. Other non-PCR-based methods that can be used in the present disclosure include, for example, strand displacement amplification (SDA) which is described in, for example Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyperbranched strand displacement amplification which is described in, for example Lage et al., Genome Research 13:294-307 (2003), each of which is incorporated herein by reference in its entirety. Isothermal amplification methods can be used with the strand-displacing Phi 29 polymerase or Bst DNA polymerase large fragment, 5′->3′ exo- for random primer amplification of genomic DNA. The use of these polymerases takes advantage of their high processivity and strand displacing activity. High processivity allows the polymerases to produce fragments that are 10-20 kb in length. As set forth above, smaller fragments can be produced under isothermal conditions using polymerases having low processivity and strand-displacing activity such as Klenow polymerase. Additional description of amplification reactions, conditions and components are set forth in detail in the disclosure of U.S. Pat. No. 7,670,810, which is incorporated herein by reference in its entirety.

Another nucleic acid amplification method that is useful in the present disclosure is Tagged PCR which uses a population of two-domain primers having a constant 5′ region followed by a random 3′ region as described, for example, in Grothues et al. Nucleic Acids Res. 21(5):1321-2 (1993), incorporated herein by reference in its entirety. The first rounds of amplification are carried out to allow a multitude of initiations on heat denatured DNA based on individual hybridization from the randomly synthesized 3′ region. Due to the nature of the 3′ region, the sites of initiation are contemplated to be random throughout the genome. Thereafter, the unbound primers can be removed and further replication can take place using primers complementary to the constant 5′ region.

In some embodiments, the amplifying serves to add one or more secondary adaptor sequences to the fully duplexed 5′ tagged target fragments to form sequencing fragments. The amplifying is accomplished by incubating a fully duplexed 5′ tagged target fragment comprising a primer sequence at each end with a secondary adaptor carrier, single nucleotides, and a polymerase under conditions sufficient to amplify the target fragments and incorporate the secondary adaptor carrier (or complement thereof), wherein the secondary adaptor carrier comprises the complement to the primer sequence and a secondary adaptor sequence.

In some embodiments, the secondary adaptor carrier comprises a primer sequence, an index sequence, a barcode sequence, a purification tag, or a combination thereof. In some embodiments, the secondary adaptor carrier comprises a primer sequence. In some embodiments, the secondary adaptor carrier comprises an index sequence. In some embodiments, the secondary adaptor carrier comprises an index sequence and a primer sequence.

In some embodiments, the fully duplexed 5′ tagged target fragments comprise a different primer sequence at each end. In such embodiments, each secondary adaptor carrier comprises the complement to one of the two primer sequences. In some embodiments, a two primer sequences are an A14 primer sequence and a B15 primer sequence.

In some embodiments, a plurality of secondary adaptors are added by amplification. In some embodiments, the secondary adaptor carriers each comprise one of two primer sequences. In some embodiments, the secondary adaptor carriers each comprise one of a plurality of index sequences. In some embodiments, the secondary adaptor carriers comprise secondary adaptors with a P5 primer sequence (SEQ ID NO: 13) and secondary adaptors with a P7 primer sequence (SEQ ID NO: 14), or their complements.

In some embodiments, the sequencing fragments are deposited on a flow cell. In some embodiments, the sequencing fragments are hybridized to complementary primers grafted to the flow cell or surface. In some embodiments, the sequences of the sequencing fragments are detected by array sequencing or next-generation sequencing methods, such as sequencing-by-synthesis.

The P5 and P7 primers are used on the surface of commercial flow cells sold by Illumina, Inc., for sequencing on various Illumina platforms. Such primer sequences are described in U.S. Patent Publication No. 2011/0059865 A1, which is incorporated herein by reference in its entirety. While the P5 and P7 primers are given as examples, it is to be understood that any suitable amplification primers can be used in the examples presented herein.

In some embodiments, the amplifying step of the method comprises PCR or isothermal amplification. In some embodiments, the amplifying step of the method comprises PCR.

In some embodiments, sequencing is performed after amplifying. In some embodiments, amplification is not performed before sequencing. A number of different sequencing are known to those skilled in the art, such as those described in U.S. Pat. Nos. 9,683,230 and 10,920,219, each of which is incorporated by reference herein in its entirety.

In some embodiments, the method comprises amplifying double-stranded cDNA fragments to prepare amplicons thereof. In some embodiments, amplicons are subjected to solid-phase reversible immobilization purification.

In some embodiments, the total reaction time from combining primers with a sample comprising RNA until purification of amplicons is 2 hours or less, 2.5 hours or less, or 3 hours or less.

As described below, amplification also may be performed to enrich for library fragments of interest as a type of target enrichment.

E. Target Enrichment

In some embodiments, library preparation is performed with one or more steps to enrich for fragments comprising targets of interest (i.e., target library fragments). Such methods can reduce the number of library fragments of low interest within the library. In this way, the user can reduce the waste of time and cost associated with sequencing of library fragments that are not of interest.

For example, a user may wish to prepare and sequence library fragments from a patient sample, wherein the library fragments of interest are those generated from nucleic acids of an agent that causes one or more infectious diseases. In such embodiments, the patient sample would generate many library fragments from nucleic acids of patient themselves (i.e., library fragments from the host), which are not of interest to the user. By enriching for sequences from one or more infectious diseases, the user could reduce sequencing of library fragments (or amplicons thereof) comprising patient-specific sequences to allow for greater depth of sequencing of library fragments (or amplicons thereof) comprising sequences from one or more infectious diseases. For example, a user may want to determine COVID-19-related sequences from a patient (in order to determine the presence or not of COVID-19 and/or to evaluate the particular COVID-19 variant) and may have less interest in sequences from the patient (i.e., the host).

In some embodiments, the infectious disease is caused by a virus, bacteria, parasite, or fungus.

Target enrichment may occur at various different steps within a library preparation and downstream steps. In some embodiments, target enrichment is performed simultaneously or just after preparation of double-stranded cDNA fragments in the library preparation method. In some embodiments, target enrichment is performed during amplification after the preparation of double-stranded cDNA fragments.

In some embodiments, double-stranded cDNA fragments are prepared with enrichment. In some embodiments, enrichment is performed during or just after tagmentation in a library preparation. In some embodiments, enrichment is performed with hybrid capture. The advantages and applications of hybrid capture to human infectious diseases are well-known (see, for example, Gaudin and Desnues, Frontiers in Microbiology, 9: Article 2924 (2018)).

In some embodiments, the hybrid capture is performed with target-specific biotinylated probes. In some embodiments, the target-specific biotinylated probes bind to sequences from one or more infectious diseases. In some embodiments, the one or more infectious diseases comprises one or more respiratory viruses. Such enrichment workflows have been described for respiratory viruses, such as COVID-19. In some embodiments, the method incorporates a viral targeting panel, as described in Enrichment workflow for detecting coronavirus using Illumina NGS systems, Illumina Document 1270-2020-002-A (2020). In some embodiments, the viral targeting panel is a Respiratory Virus Oligo Panel (RVOP, Illumina). Multiple versions of the RVOP are known in the literature. FIGS. 26-30B present data on experiments performed with RVOP enrichment (either Version 1 or Version 2).

Target enrichment may also be performed during amplification of double-stranded cDNA fragments. In some embodiments, a method of library preparation further comprises amplifying the double-stranded cDNA fragments to prepare amplicons. In some embodiments, the amplifying is performed with target-specific primers. In some embodiments, the target-specific primers bind sequences from one or more infectious diseases. In some embodiments, the one or more infectious diseases comprise one or more respiratory viruses.

### EXAMPLES

### Example 1. Overview of Isothermal Method of Ds-cDNA Preparation

An isothermal method can be used to prepare ds-cDNA from RNA in a single isothermal reaction. A number of different ways of performing this reaction will be presented, wherein a similar method is performed after binding of primers to RNA (such as random hexamers). After primer binding, a reaction mix (which may be termed a “master mix”) can be added comprising the following components:
1. A reverse transcriptase to form a first strand of cDNA;
2. RNAse H to nick RNA and generate priming sites for second strand cDNA synthesis;
3. A DNA polymerase, with either strand displacement activity or 5′-3′ exonuclease activity, to generate a second strand cDNA; and
4. dNTPs, which are shared between the reverse transcriptase and the DNA polymerase to generate first and second strands.

Such a method may be termed a “single-step” or “1-step” method (i.e., the present method), as first and second strand cDNA occur concomitantly in a single reaction vessel. An appropriate formulation to enable single-step double-stranded cDNA synthesis may be needed. The hypothesized mechanism of this reaction is shown in FIG. 3.

### Example 2. Method of Mesophilic Ds-cDNA Preparation

A method of mesophilic ds-cDNA preparation was evaluated in the context of the Illumina RNA Prep with Enrichment (L) product (See RNA Prep with Enrichment (L) Tagmentation Reference Guide, Document #1000000124435 v02, Illumina, 2020 (“Document 1000000124435”). In basic experiments, 10-12 ng of Universal Human Reference RNA (UHR, Agilent PN 740000) enriching with the Illumina TruSight® RNA Pan-Cancer Panel kit. In some cases, ˜3.5 kb genomic RNA derived from bacteriophage MS2 (MS2, Roche PN 10165948001, GenBank accession NC 001417.2) was used as control material to evaluate library preparation performance. Libraries were prepared with ˜10-12 ng total RNA input, using either pure UHR, MS2, or a mixture of UHR and MS2 RNA (80% UHR/20% MS2 by mass) as noted below.

Equal volumes of sample RNA and EPH3 (Illumina) buffer (8.5 μl each) were mixed, heated to 65° C. for 5 minutes, and then cooled to 4° C. in a thermal cycler. This step allowed for hybridization of primers comprised in the EPH3 buffer to the RNA.

Following primer binding incubation, 33 μl of the ˜1.5× “single-step” cDNA synthesis buffer (i.e., a cDNA synthesis master mix) was added directly to the 17 μl of the RNA+EPH3 mix. The single step formulation was incubated for either 10 min, 20 min, 30 min, 45 min, or 60 minutes to determine how incubation time affects performance. The single-step cDNA synthesis reaction may be termed the “present method” in Figures.

cDNA was generated from 12 ng of 80% UHR/20% MS2 RNA using the single-step procedure, followed by tagmentation with eBLT and enrichment with the TruSight® RNA Pan-Cancer Panel kit panel as described in the Illumina RNA Prep with Enrichment (Document 1000000124435 (2020)). Sequenced libraries were compared to similar libraries generated from 10 ng or 100 ng UHR using Illumina RNA Prep with Enrichment with enrichment by RNA Pan-Cancer Oligos, included as part of TruSight® RNA Pan-Cancer Panel kit (See TruSight® RNA Pan-Cancer Panel Reference Guide, Illumina Document #1000000001632-v01 (2016)). Data was analyzed using the Illumina BaseSpace Sequence Hub (BSSH) RNA-Seq Alignment App v. 1.1.1.

A. Analysis of Percentage of Duplicate Reads

The percentage of duplicate reads is a measure of the conversion of sample into library. A lower percentage of duplicate reads is preferred. In terms of duplicates, results suggest the single-step method has equivalent to better performance than standard procedures (FIG. 4). In the single-step workflow, incubation times between 10 minutes and 60 minutes had little impact on performance.

B. Analysis of Insert Size

As expected according to the model (FIG. 3), the present single-step method produces library fragments that are shorter than the standard procedure with Illumina RNA Prep with Enrichment (FIG. 5). Insert size means of ˜150-170 bp generated with the present single-step method are acceptable for most RNA-Seq applications. Incubation time (down to 10 minutes) had little performance impact in the single-step workflow.

C. Median CV of Coverage

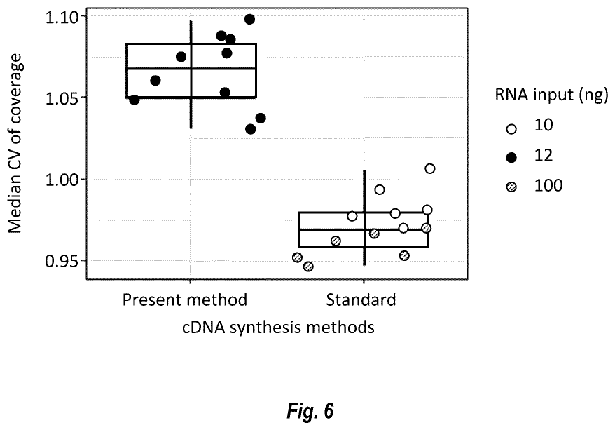

The median coefficient of variance (CV) of coverage is reflective of library quality, wherein more uniform conversion of sample into library results in a lower CV. Results are shown in FIG. 6. Incubation times had little impact on the single-step workflow, as measured at 10-, 20-, 30-, 45-, and 60-minutes incubation.

D. Gene Expression Correlation

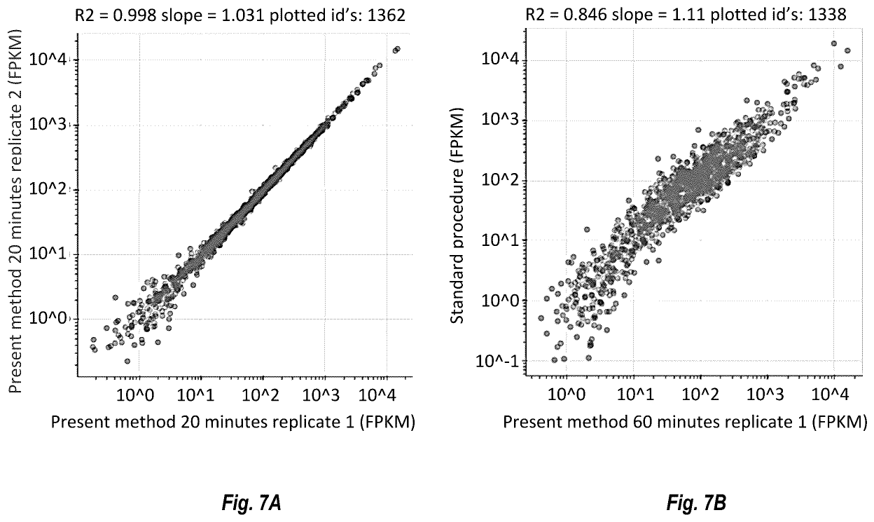

An important feature of RNA-Seq products is high reproducibility of gene expression estimation between technical replicates. Gene expression reproducibility is high for technical replicates of the single-step method with only a 20-minute enzymatic incubation (FIG. 7A, wherein shown exemplary data have an R² value of 0.998). Similar results were achieved with a 10-minute incubation (not shown).

As a quantitative assay, RNA-Seq is highly sensitive to changes in protocol and reagents. Unsurprisingly, the single-step method shows less correlation to the standard Illumina RNA Prep with Enrichment protocol as displayed in an example comparison (FIG. 7B). While this cross-procedure comparison displays a lower R² of fragments per kilobase per million mapped reads (FPKM, a measure to normalize for sequencing depth and gene length) value (0.846), the Spearman p (rank order) is high (p=0.934), indicating that the single-step method retains quantitative information.

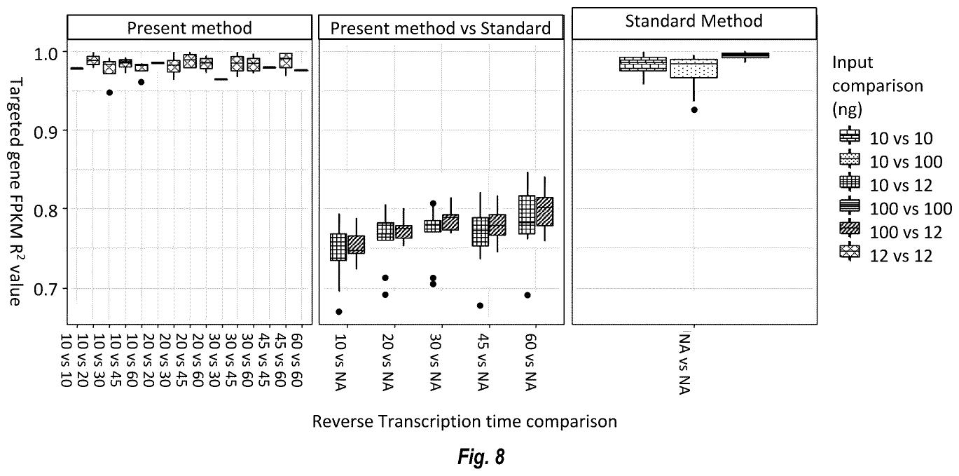

Boxplots comparing the R² values of FPKM comparisons across multiple library preparations using both standard Illumina RNA Prep with Enrichment cDNA procedures and the single-step cDNA method with different incubation times indicate high reproducibility within each method and, as expected, lower concordance between the two methods (FIG. 8).

E. Coverage Characteristics of MS2 Control RNA

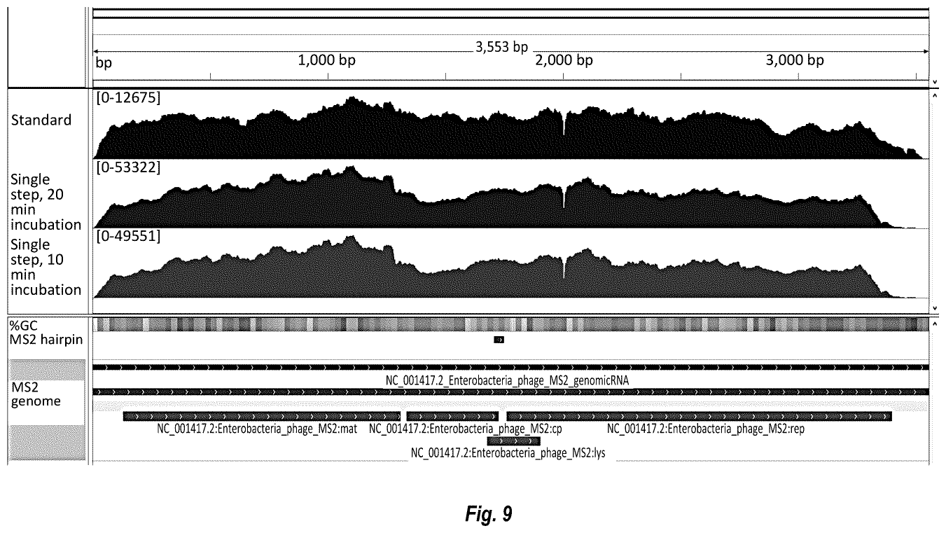

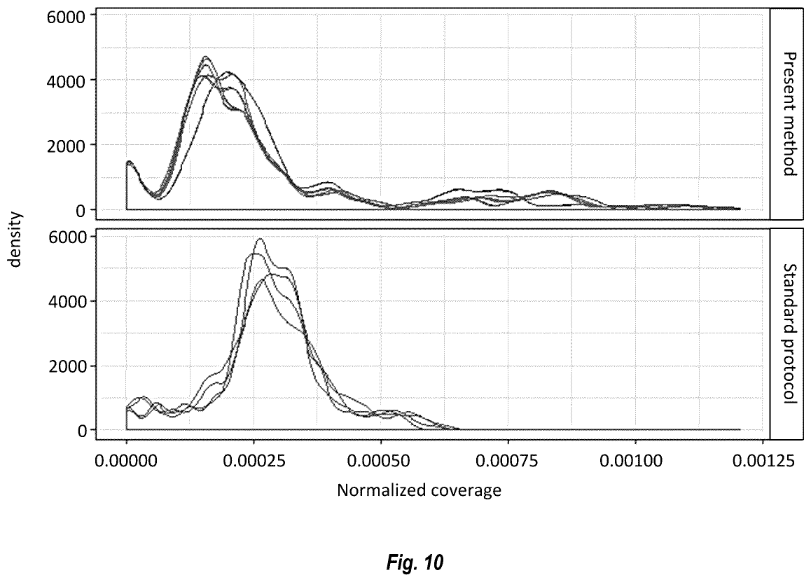

MS2 genomic RNA is a useful template when exploring alternative library preparations because it is easy to visualize and analyze assay performance without the complexity of human transcriptomics and enrichment analyses. Here, bases within the MS2 genome were evaluated for their raw read coverage, allowing calculation of a CV of coverage across the MS2 genome and a read-depth normalized value of coverage for comparison across experiments. Visually, read coverage of MS2 is similar between the standard and single-step protocols (FIG. 9), though data suggests that the single-step protocol has a slightly higher CV of coverage (FIG. 10 and Table 6).

TABLE 6
CV of coverage for the new single-step protocol and comparative
Illumina RNA Prep with Enrichment protocol

| Protocol | Number of replicates | Mean CV of coverage |
|---|---|---|
| Single-step | 10 * | 1.02 |
| Illumina RNA Prep with | 4 ** | 0.40 |
| Enrichment | | |

* All replicates were of 80% UHR, 20% MS2 mixtures. 12 ng input.
** 2 libraries were 100% MS2, 2 libraries were 80% UHR/20% MS2 mixture. Inputs varied from 10 ng to 100 ng.

Accordingly, the present single step protocol provided sufficient cDNA quality with a faster preparation time.

### Example 3. Method of Thermostable Ds-cDNA Preparation

Coordinated synthesis of double-stranded cDNA can also occur using an alternative formulation composed of thermostable enzymes, rather than their mesophilic counterparts (Table 2). Such a formulation thermostable enzymes may be referred to as a thermostable master mix.

Equal volumes of sample RNA and EPH3 (Illumina) buffer (8.5 μl each) were mixed, heated to 65° C. for 5 minutes, and then cooled to 4° C. in a thermal cycler. This step allowed for hybridization of primers comprised in the EPH3 buffer to the RNA. Then 15 μl of the reaction with hybridized primers was added to thermostable enzyme and buffer components for single-step cDNA preparation.

The thermostable formulation was performed isothermally at ˜50° C. Tagmentation of the resulting ds-cDNA results and enrichment with the TruSight® RNA Fusion Panel (as described in TruSight RNA® Fusion Panel Protocol Guide, Illumina Document #1000000009155 v00(2016)) results in quality libraries (FIG. 11). As shown in FIG. 11, the thermostable formulation produced insert sizes of approximately 250 bp, which is similar to the insert size produced with standard Illumina RNA Prep with Enrichment methodologies (for example, approximately 210 bp as shown in FIG. 5). Libraries are quantitative with both reasonable replicate-to-replicate technical reproducibility and a reasonable concordance with methods using Illumina RNA Prep with Enrichment (FIGS. 12A and 12B). A thermostable formulation may be preferable in cases where RNA secondary structure is of a concern and may otherwise inhibit first strand cDNA synthesis occurring at 37° C.

In this experiment, incubation was performed for 60 minutes, though much shorter incubation times are possible. Furthermore, replacement of MMLV reverse transcriptase with alternative thermostable reverse transcriptase derived from retrotransposons may improve performance and assay speed.

### Example 4. Summary of 2-Step Versus 1-Pot cDNA Preparations

FIG. 13 provides an overview of a representative 1-pot tagmentation protocol. Representative 2-step and 1-step cDNA preparation protocols are shown in FIG. 14A, with option to add BLTs to either protocol for preparing a library of cDNA fragments. FIG. 14B highlights outline how the 1-step cDNA preparation protocol (also referred to herein as the present method) shortens the reaction time by approximately an hour and also avoids multiple temperature changes. While ST2 (stop tagmentation buffer) is not required if a user only wants to prepare cDNA, it is included as it can be used to stop tagmentation if BLTs are included in the protocols outlined in FIG. 14B for library preparation.

FIG. 14C shows protocols for a current method of 2-step cDNA preparation followed by separate tagmentation, along with shorter protocols for 1-step cDNA (i.e., present method) followed by separate tagmentation or for a 1-pot library preparation with combined cDNA and library preparation. The 1-pot library can save over an hour compared to current methods.

To compare cDNA yields, cDNA prepared with 1-pot or 2-step cDNA protocols was purified with solid-phase reversible immobilization (SPRI) beads and eluted. As shown in FIG. 4, both the present (1-step) and 2-step (Standard) protocols yielded cDNA.

### Example 5. Comparison of 1-Pot Library Preparation Versus Methods with Separate Tagmentation

A variety of different library preparation protocols summarized in FIG. 14C were evaluated.

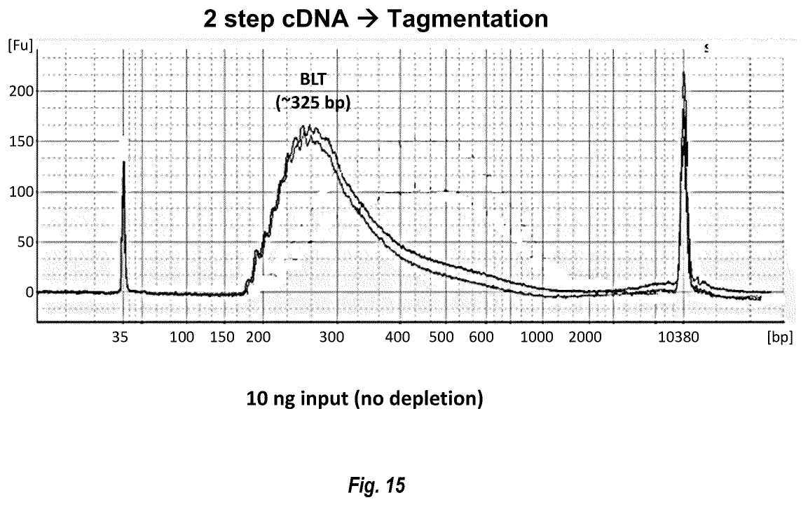

Experiments evaluated fragments generated with BLTs after a 2-step cDNA preparation with 10 ng input RNA (without rRNA depletion). These results showed that BLTs successfully generated fragments with a size of approximately 325 base pairs after a 2-step cDNA preparation (FIG. 15).

The 1-pot library preparation allowed for preparation of a library of double-stranded DNA fragments from a starting sample comprising RNA. In this protocol, a sample comprising RNA was mixed with a mix comprising primers (EPH3). This reaction mix (with a total volume of 17 μl) was:
7.5 μl nuclease-free water
1 μl of 10 ng/μl UHR total RNA
8.5 μl EPH3

The reaction mix was placed in a thermocycler with a heated lid, and the reaction was heated to 65° C. for 5 minutes, followed by a hold at 4° C.

The plate was removed from the thermocycler and 33 μl of a 1-pot master mix was added to generate a total volume of 50 μl. The 1-pot master mix comprised a balance of enzyme units in the range discussed in Section I.C, along with BLTs. The enzymes in the 1-pot master mix used were E coli DNA polymerase I (within range of 0.04 U/μl to 0.37 U/μl), RNAse H (within range of 0.004 U/μl to 0.04 U/μl), and Protoscript II reverse transcriptase (within range of 0.32 U/μl to 4.8 U/μl). The reaction was mixed well.

A total of 10 μl of BLTs (Illumina Catalog #20024594) was aliquoted into well of a plate. The plate was spun down to collect BLTs at the bottom of the wells. The plate was then placed on a magnet for approximately 2 minutes. Then approximately 10 μl of supernatant was removed. This step removed buffer supplied with the BLTs.

BLTs were then resuspended with the 50 μl sample together with 1-pot master mix. This plate was incubated at 37° C. for 1 hour in a thermocycler with heated lid set to 45° C. Then, 10 μl ST2 buffer was added to stop the tagmentation reaction. The plate was allowed to stand at room temperature for approximately 5 minutes. The plate was then placed on a magnet for 2 minutes, after which supernatant was removed and discarded. Beads were resuspended with 100 μl TWB wash buffer. The washing with TWB was repeated for a total of 3 washes and then excess TWB was removed.

PCR was then performed. To each sample, 20 μl enhanced PCR mix (EPM, Illumina) buffer and 20 μl nuclease-free water was added. Then, 10 μl unique dual index PCR primers were added (for total volume of 50 μl). The plate was placed in a thermocycler and amplified for 15 cycles of 98° C. for 10 seconds, 60° C. for 30 seconds, and 72° C. for 30 seconds.

The samples were purified with 81 μl Illumina Purification Beads (IPB). The DNA was eluted from the beads with 30 μl resuspension buffer (RSB, Illumina). Library yield was quantified using Qubit (Thermo Fisher Scientific).

Results from different protocol conditions are shown in FIGS. 16A-16D. The 1-pot library preparation (1-pot combined cDNA and tagmentation reaction, FIG. 16C) produced similar results to the 2-step cDNA preparation with separate tagmentation (FIG. 16A) and 1-pot cDNA preparation with separate tagmentation (FIG. 16B). The comparison of results with different protocols is summarized in FIG. 16D. In summary, the 1-pot library preparation produced similar library yield with a significantly faster and simpler protocol as compared to other tested protocols.

FIG. 17A shows the results with 100 ng UHR and the 1-pot library preparation (comprising cDNA preparation and tagmentation in a single reaction). In comparison, FIG. 17B shows the results of a no template control (NTC, with no starting RNA), and FIG. 17C shows the results of a control lacking reverse transcriptase (RT). These results indicate that the 1-pot library results are based on successful preparation of double-stranded cDNA and tagmentation of this cDNA.

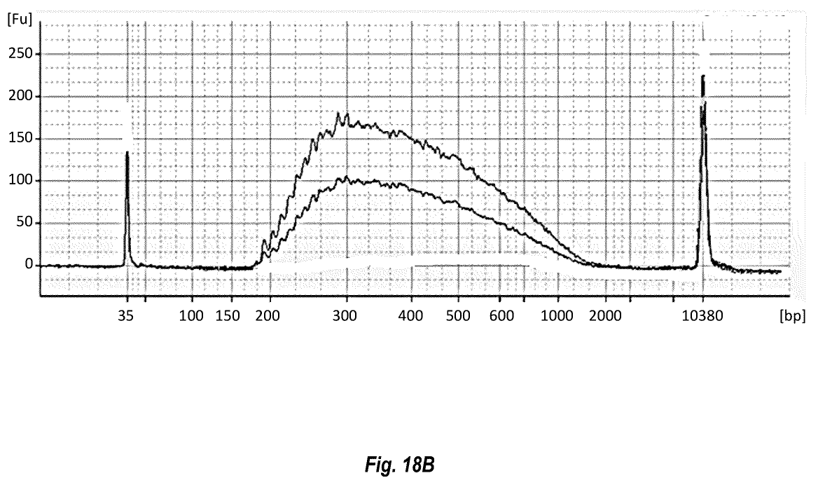

Both for starting samples comprising 100 ng of RNA (FIG. 18A) and comprising 10 ng of RNA (FIG. 18B), BLTs successfully prepared library fragments.

### Example 6. Evaluation of Library Preparations with rRNA Depletion

Experiments evaluated different library preparations using BLTs with rRNA depletion using standard conditions for Illumina® Ribo-Zero Plus rRNA Depletion Kit before primer binding. For 100 ng samples, 13 cycles of PCR were used. For 10 ng samples, 15 cycles of PCR were used.

Results of experiments with rRNA depletion are shown in FIGS. 19A-19B. The yield with the 1-pot library preparation were lower than with other preparations, which may be likely due to the relatively low amount of starting RNA after the rRNA depletion. Control experiments without reverse transcriptase (No RVT) did not show library yield.

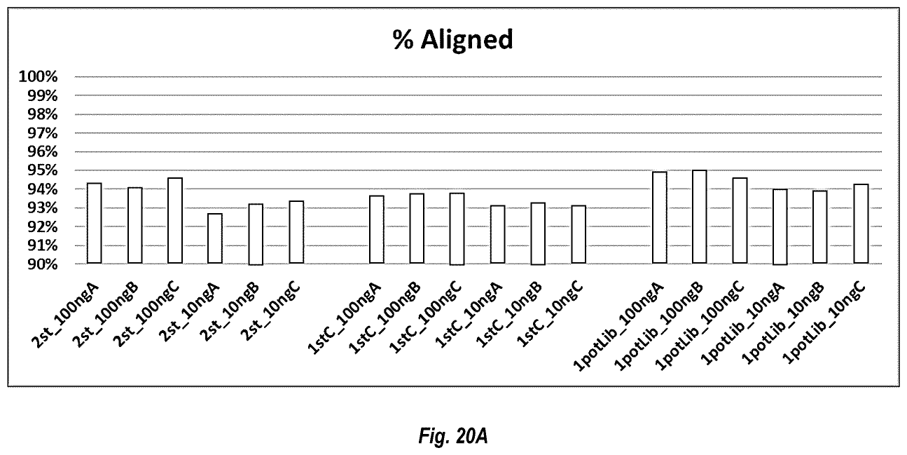

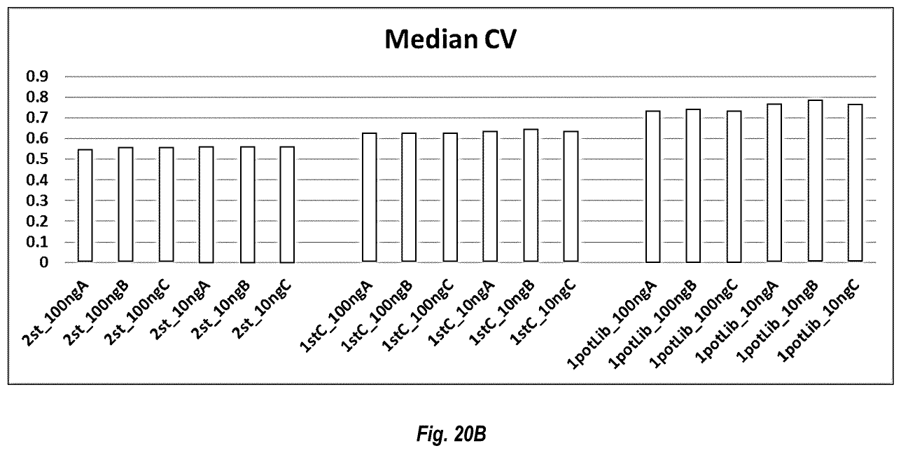

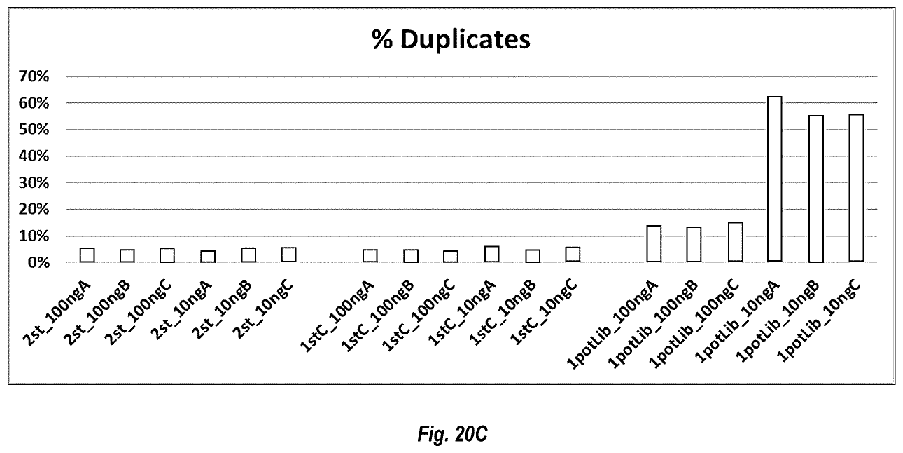

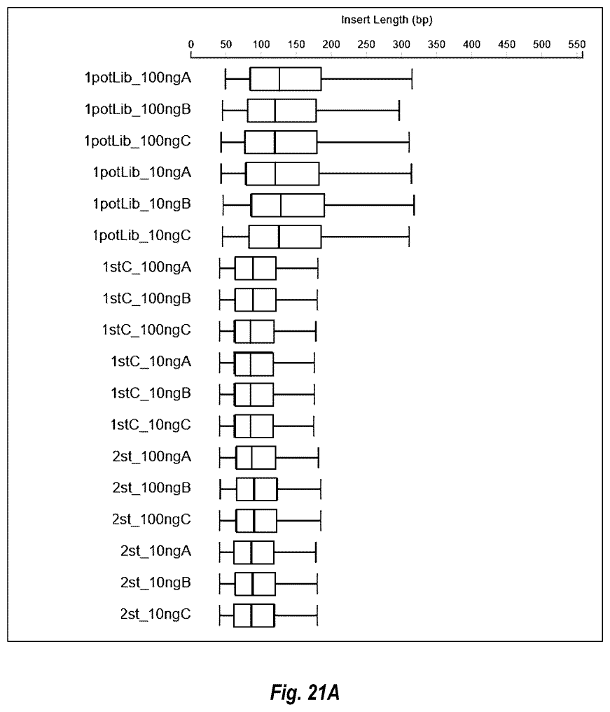

The alignment and general performance metrics are shown in FIG. 20A-20C. The percentage alignments were all approximately 94-95% (FIG. 20A). The median coefficient of variation of coverage (median CV) and the percentage duplicates were higher for the 1-pot library preparations as compared to protocols with separate tagmentation reactions (FIGS. 20B and 20C). These results may indicate relatively lower efficiency of tagmentation with the 1-pot library preparation as compared to other conditions. The percentage abundance was approximately 1-2% for all preparations (data not shown).

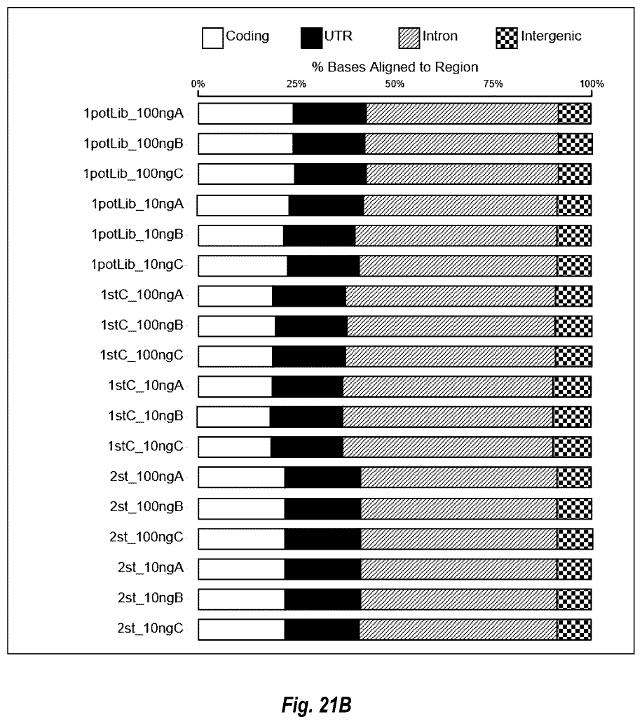

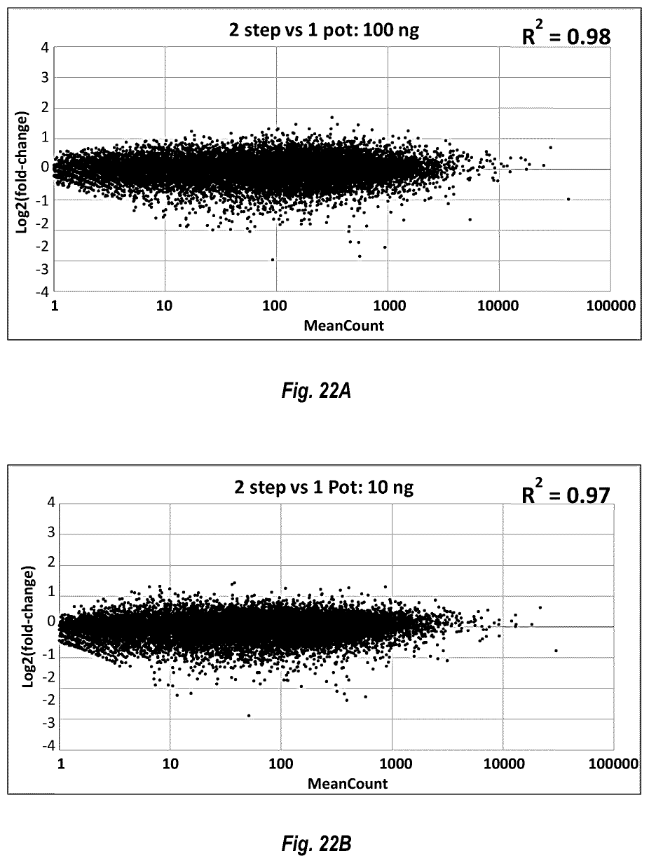

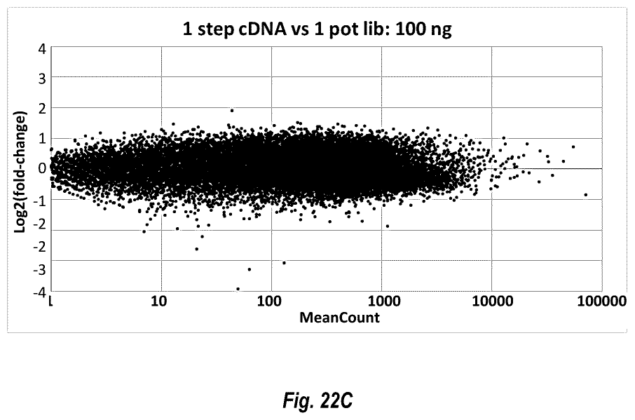

Insert length and alignment distribution were similar for all library preparations (FIGS. 21A-21B). Further, gene expression correlations were good between control library preparation (i.e., 2-step cDNA preparation with separate tagmentation) and the 1-pot library preparation with both 100 ng (FIG. 22A) and 10 ng (FIG. 22B) starting material. Gene expression correlations were also good when comparing the 1-pot library preparation to a 1-pot cDNA protocol with separate tagmentation (FIG. 22C).

Some outlier genes with relatively larger differences in expression with the 1-pot library preparation versus a 2-step cDNA preparation with separate tagmentation are listed in Table 7; however, these genes represent only a small amount of the total library.

TABLE 7
Gene expression correlations for 1-step cDNA and separate tagmentation
reaction versus 1-pot library reaction (100 ng starting RNA)

| | | | log2 (Fold |
|---|---|---|---|
| Gene | Status | Mean Count | Change) |
| HBA2 | OK | 49.8 | −3.91 |
| HBA1 | OK | 62.9 | −3.28 |
| HBB | Outlier | 130 | −3.08 |
| RNU12 | OK | 21 | −2.62 |
| SCARNA7 | OK | 23.5 | −2.22 |
| HBG2 | Low | 6.98 | −2.05 |
| SNORD45C | OK | 14 | −1.95 |
| SNORA32 | OK | 21.7 | −1.88 |
| PTMA | OK | 1,130 | −1.87 |

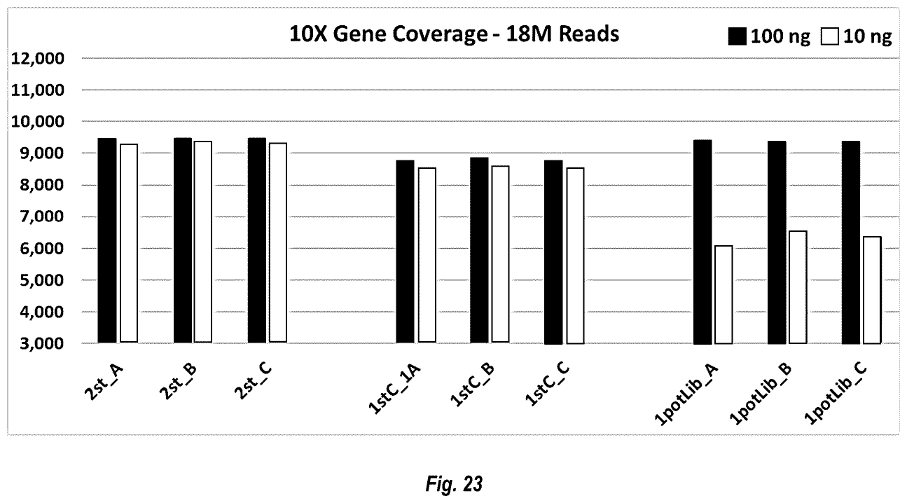

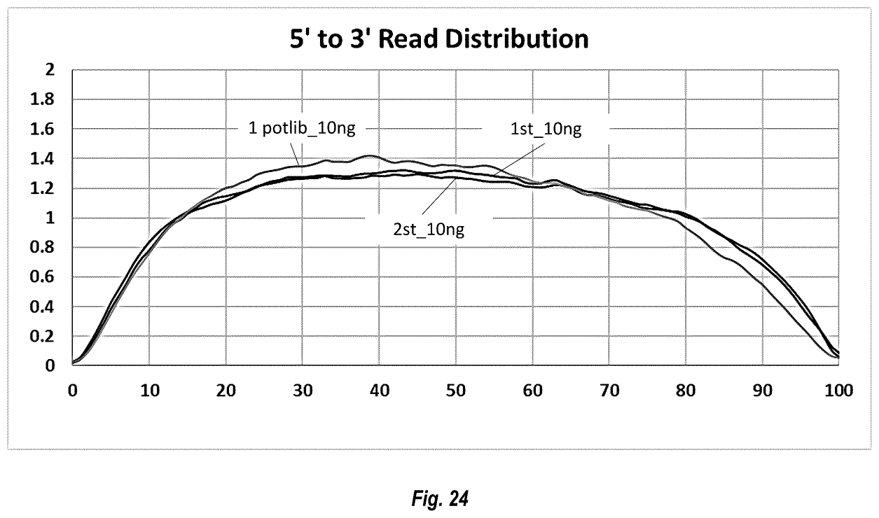

The number of genes detected at 10× coverage (18 million reads) was lower for the 1-pot library preparation protocol with 10 ng starting RNA, but not different with 100 ng starting RNA (FIG. 23). These results indicate that the 1-pot library preparation may result in relatively inefficient tagmentation at lower input ranges. The read distribution was normal for all conditions, suggesting no 5′ or 3′ bias in the library preparations (FIG. 24).

These results indicate that tagmentation efficiency may be a limiting factor in determining the yield of 1-pot library preparations.

To test the effects of improving tagmentation efficiency, reactions were performed with RNA Prep with Enrichment and Ribo-Zero Plus rRNA depletion kit (Illumina), using conditions with nearly double the amount of MgCl₂ (8.25 mM MgCl₂ versus standard 4.3 mM MgCl₂), and these conditions improved yields (data not shown). These results indicate that 1-pot library preparation compositions with higher magnesium concentrations can increase yield of library fragments, likely by increasing transposase efficiency.

Similarly, increasing temperature at the end of the reaction incubation for a 1-pot library preparation may produce higher library yields. Higher reaction temperatures may increase transposase activity, as 55° C. is known to be an optimum temperature for Tn5.

### Example 7. Optimization of 1-Pot Library Preparation Conditions

Results (such as described in Example 5) showed that the 1-pot library preparation with simultaneous cDNA synthesis and tagmentation could robustly produce robust libraries. A variety of different conditions were next assessed to potentially improve library yield, focusing on protocol modifications to attempt to increase tagmentation efficiency with 1-pot library preparation, given its ability to decrease library preparation time and hands-on step (as shown in FIG. 25). It should be noted, however, that for many applications (such as library preparations with enrichment) any potential reduced library preparation efficiency with a 1-pot protocol will likely not impact downstream steps such as sequencing.

Enrichment experiments were performed with a variety of different protocols, using 10 ng UHR as “host RNA” with synthetic controls. The synthetic controls (“Twist”) were 0, 1k, 10, or 100k Twist Control Respiratory Virus Controls (Twist Bioscience) that were spiked into the UHR sample. The enrichment was performed using the Respiratory Virus Oligos Panel V1 (RVOP, Illumina). Protocols for data shown in FIG. 26 included:
Standard library preparation (LP) with enrichment (i.e., 2-step cDNA preparation followed by tagmentation as control, StdLP)
1-pot LP: 37° C. for 1 hr (4 mM Mg²⁺)—conditions outlined in Example 5 (1Pot)
1-pot LP: 37° C. for 45 minutes, followed by 55° C. for 15 min (55C)
1-pot LP: 37° C. for 1 hour, increase Me to 8 mM final (Mg)
1-pot LP: 37° C. for 1 hr, skip addition ST2 (stop tagmentation solution comprising SDS) and washes (NoST2).

Results in FIG. 26 show that the standard LP (StdLP group) had the best yield. The addition of an incubation at 55° C. (55C group) or extra Mg2+ (Mg) improved yield over the original 1-pot LP protocol (1Pot). Skipping SDS and washes (NoST2) was detrimental to yield.

Coverage was also assessed by determining the median coverage of the Twist control at 1 million reads with Respiratory Virus Oligos Panel Version 1 (Illumina) (FIG. 27). While 1-pot library preparation protocols did not generally perform as well as a standard library preparation protocol, the addition of extra Mg²⁺ (to a final concentration of 8 mM) gave a significant boost to the performance of the 1-pot library preparations. The largest increase in activity seemed to be due to the increase in Mg²⁺ concentration, and little coverage when seen after skipping SDS and washes (NoST2 samples in FIG. 27). Generally, when target enrichment is performed during library preparation, a user may not need high coverage of the desired target sequences as for an unenriched library. Accordingly, coverage as shown in FIG. 27 may be sufficient for many types of sequencing analysis.

Experiments were next done to combine conditions that may boost the performance of 1-pot library preparations (FIG. 28). The tested groups included some combined protocols that had incubations at 55° C. and a 8 mM Mg²⁺ concentration, and the combined protocols had higher library yield than the 1-pot LP at 37° C. with 4 mM Mg²⁺.

Better coverage was seen with enrichment with Respiratory Virus Oligos Panel V2 (Illumina) (FIG. 29) as compared to Respiratory Virus Oligos Panel Version 1 (Illumina) (FIG. 27). While methods including a 15-minute incubation at 55° C. generally had better results than incubation at 37° C., there was little difference between results for different times of incubation when all reactions had a 8 mM Mg²⁺ concentration (FIG. 29). These data indicate that library preparations could be performed in as little as 30 minutes (e.g., 15 minutes at 37° C. and 15 minutes at 55° C.).

Library preparation conditions were also investigated for ribosomal RNA (rRNA) depleted samples for samples with 10 ng UHR. The different library preparation conditions were:
37° C. for 1 hour
55° C. for 15 minutes or 30 minutes after 37° C. incubation
Extra Mg²⁺ conditions (8 mM)

FIG. 30A shows that adding incubations of 15 minutes (3715) or 30 minutes (3730) at 55° C. at 8 mM Mg²⁺ dramatically reduced the percentage of duplicates as compared to the standard 1-pot library preparation at 37° C. for 1 hour with 4 mM Mg²⁺. For example, the duplicates dropped from approximately 50% for 1-pot library preparation in 4 mM Mg²⁺ at 37° C. for 1 hour to approximately 6% for 1-pot library preparation with the entire reaction run in 8 mM Mg²⁺ for 1 hour at 37° C. followed by a 55° C. incubation for 15 (3715) or 30 minutes (3730).

The number of genes detected after different library preparation was also evaluated (FIG. 30B). Under standard 1-pot library preparation conditions (4 mM Mg²⁺ for 1 hour at 37° C.) approximately 2200 fewer genes were detected as compared to a standard 2-step cDNA preparation followed by tagmentation (2st). With the 3715 and 3730 protocols, however, only approximately 750 fewer genes were detected as compared to the 2st protocol. Thus, protocols with 55° C. incubations and higher Mg²⁺ concentrations showed the highest number of genes detected for the 1-pot protocols, in comparison to the standard 2-step cDNA preparation followed by tagmentation protocol.

Accordingly, for measures of duplicates and number of genes detected, 1-pot protocols were improved by incorporating a 55° C. incubation and a higher Mg²⁺ concentration (such as 8 mM). These improvements were seen with these conditions for both rRNA depleted samples and for enriched samples. These results may indicate that tagmentation efficiency is the limiting step for library yields for 1-pot protocols.

When a user does not require a maximum yield for library preparation of a given sample, 1-pot protocols may be preferred for their shorter times, down to 30 minutes total for the library preparation. Further, the user may also prefer a 1-pot protocol to reduce the number of hands-on steps. For example, a 1-pot protocol can omit a cleanup step with SPRI beads between cDNA synthesis and tagmentation (as shown in FIG. 25). 1-pot protocols can also eliminate sample loss that is inherent in steps that require sample pipetting.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A composition for preparing double-stranded cDNA from RNA by an isothermal reaction comprising:
a. a reverse transcriptase;
b. an RNA nickase;
c. a DNA polymerase with strand displacement activity or 5′-3′ exonuclease activity; and
d. dNTPs.

2. The composition of claim 1, wherein the activity of the reverse transcriptase is greater than the activity of the RNA nickase.

3. The composition of claim 1, wherein the reverse transcriptase and the RNA nickase are comprised in a single enzyme.

4. The composition of claim 1, wherein the reverse transcriptase and the DNA polymerase are comprised in a single enzyme with both RNA-dependent and DNA-dependent polymerase activity.

5. The composition of claim 4, wherein the single enzyme reduces competition between the reverse transcriptase and the DNA polymerase.

6. The composition of claim 1, wherein the DNA polymerase has strand displacement activity or the DNA polymerase has 5′-3′ exonuclease activity.

7. (canceled)

8. The composition of claim 1, wherein:
(a) the reverse transcriptase is a polymerase with RNA-dependent DNA polymerase activity, optionally wherein the reverse transcriptase is Moloney Murine Leukemia Virus (MMLV) reverse transcriptase, a reverse transcriptase derived from a retrotransposon, or a Group II intron reverse transcriptases;
(b) the RNA nickase is RNAse H;
(c) the RNAse H is from Thermus thermophilus; and/or
(d) the DNA polymerase is E. coli DNA polymerase I or Bst DNA polymerase.

9.-11. (canceled)

12. The composition of claim 1, wherein the reverse transcriptase, the RNA nickase, and/or the DNA polymerase are mesophilic enzymes.

13. The composition of claim 12, wherein:
(a) the mesophilic enzymes have activity at 37° C.-49° C., optionally wherein the mesophilic enzymes have activity at 37° C.;
(b) the mesophilic reverse transcriptase is MMLV reverse transcriptase;
(c) the mesophilic RNA nickase is E. coli RNAse H; and/or
(d) the mesophilic polymerase is E. coli DNA polymerase I.

14.-17. (canceled)

18. The composition of claim 1, wherein the reverse transcriptase, the RNA nickase, and/or the DNA polymerase are thermostable enzymes.

19. The composition of claim 18, wherein:
(a) the thermostable enzymes have activity at 50° C.-72° C., optionally wherein the thermostable enzymes have activity at 50° C.;
(b) the thermostable reverse transcriptase is a thermostable variant of MMLV reverse transcriptase or a thermostable reverse transcriptase derived from a retrotransposon or a Group II intron reverse transcriptase;
(c) the thermostable RNA nickase is RNAse H from Thermus thermophilus; and/or:
(d) the thermostable DNA polymerase is Bst DNA polymerase.

20.-23. (canceled)

24. The composition of claim 1, wherein:
(a) the RNA is bound to primers before preparing the double-stranded cDNA;
(b) the composition further comprises one or more additives chosen from DTT, BSA, Tris pH 7.5, KCl, and/or MgCl₂; and/or
(c) the composition has a lower units/μl of the RNA nickase as compared to the units/μl of the reverse transcriptase and/or DNA polymerase.

25. (canceled)

26. (canceled)

27. The composition of claim 1, wherein the composition further comprises an RNA nickase inhibitor, optionally wherein the RNA nickase inhibitor lowers the activity of the RNA nickase.

28. (canceled)

29. The composition of claim 1, wherein the units/μl of the RNA nickase and the DNA polymerase in the composition overlap.

30. The composition of claim 1, wherein the activity of the DNA polymerase in the composition is 2-fold to 100-fold higher than the activity of the RNA nickase in the composition.

31. The composition of claim 1, wherein the activity of the of the reverse transcriptase in the composition is 10-fold to 1,000-fold higher than the activity of the RNA nickase in the composition.

32. The composition of claim 1, wherein the reverse transcriptase activity in the composition is 0.32 U/μl to 4.8 U/μl.

33. The composition of claim 1, wherein the DNA polymerase activity in the composition is 0.04 U/μl to 0.37 U/μl.

34. The composition of claim 1, wherein the RNA nickase activity in the composition is 0.004 U/μl to 0.04 U/μl, greater than 0.04 U/μl, or 0.05 U/μl to 0.3 U/μl.

35. (canceled)

36. (canceled)

37. A method of preparing double-stranded cDNA comprising:
a. combining primers with a sample comprising RNA and allowing binding of the primers to an RNA; and
b. combining the sample with a composition comprising:
i. a reverse transcriptase;
ii. an RNA nickase;
iii. a DNA polymerase with strand displacement activity or 5′-3′ exonuclease activity; and
iv. dNTPs; and
c. preparing double-stranded cDNA by an isothermal reaction.

38.-65. (canceled)

66. A composition for preparing a library of double-stranded cDNA fragments from RNA comprising:
a. a reverse transcriptase;
b. an RNA nickase;
c. a DNA polymerase with strand displacement activity or 5′-3′ exonuclease activity;
d. dNTPs; and
e. a transposome complex, wherein the transposome complex comprises:
i. a transposase;
ii. a first transposon comprising a transposon end sequence; and
iii. a second transposon comprising a sequence fully or partially complementary to the transposon end sequence.

67.-108. (canceled)

109. A method of preparing a library of double-stranded cDNA fragments comprising:
a. combining primers with a sample comprising RNA and allowing binding of the primers to an RNA; and
b. combining the sample with the composition of claim 66 and (i) preparing double-stranded cDNA by an isothermal reaction and (ii) preparing double-stranded cDNA fragments.

110.-201. (canceled)


SEQUENCE LISTING (24 sequences)

SEQ ID NO: 1 - DNA, 19 bp, synthetic construct
  source 1..19: mol_type = other DNA
agatgtgtataagagacag

SEQ ID NO: 2 - DNA, 19 bp, synthetic construct
  source 1..19: mol_type = other DNA
ctgactcttatacacaagt

SEQ ID NO: 3 - DNA, 19 bp, synthetic construct
  source 1..19: mol_type = other DNA
ctgtctcttgatcagatct

SEQ ID NO: 4 - DNA, 19 bp, synthetic construct
  source 1..19: mol_type = other DNA
ctgtctcttatacacatct

SEQ ID NO: 5 - DNA, 19 bp, synthetic construct
  misc_feature 16: note = mod_base - uracil
  source 1..19: mol_type = other DNA
agatgtgtataagagtcag

SEQ ID NO: 6 - DNA, 19 bp, synthetic construct
  source 1..19: mol_type = other DNA
ctgactcttatacacatct

SEQ ID NO: 7 - DNA, 19 bp, synthetic construct
  misc_feature 17: note = mod_base - uracil
  source 1..19: mol_type = other DNA
agatgtgtataagagatag

SEQ ID NO: 8 - DNA, 19 bp, synthetic construct
  source 1..19: mol_type = other DNA
ctatctcttatacacatct

SEQ ID NO: 9 - DNA, 19 bp, synthetic construct
  misc_feature 18: note = mod_base - uracil
  source 1..19: mol_type = other DNA
agatgtgtataagagactg

SEQ ID NO: 10 - DNA, 19 bp, synthetic construct
  source 1..19: mol_type = other DNA
cagtctcttatacacatct

SEQ ID NO: 11 - DNA, 14 bp, synthetic construct
  source 1..14: mol_type = other DNA
tcgtcggcagcgtc

SEQ ID NO: 12 - DNA, 15 bp, synthetic construct
  source 1..15: mol_type = other DNA
gtctcgtgggctcgg

SEQ ID NO: 13 - DNA, 29 bp, synthetic construct
  misc_feature 23: note = mod_base - uracil
  source 1..29: mol_type = other DNA
aatgatacggcgaccaccgagatctacac

SEQ ID NO: 14 - DNA, 24 bp, synthetic construct
  source 1..24: mol_type = other DNA
caagcagaagacggcatacgagat

SEQ ID NO: 15 - DNA, 19 bp, synthetic construct
  modified_base 1: mod_base = OTHER; note = 5' phosphate
  modified_base 19: mod_base = OTHER; note = 3' Biotin
  source 1..19: mol_type = other DNA
ctgtctcttatacacatct

SEQ ID NO: 16 - DNA, 19 bp, synthetic construct
  modified_base 19: mod_base = i
  source 1..19: mol_type = other DNA
agatgtgtataagagacan

SEQ ID NO: 17 - DNA, 19 bp, synthetic construct
  misc_feature 19: note = mod_base - uracil
  source 1..19: mol_type = other DNA
agatgtgtataagagacat

SEQ ID NO: 18 - DNA, 19 bp, synthetic construct
  modified_base 16: mod_base = OTHER; note = 8-oxo-guanine
  source 1..19: mol_type = other DNA
agatgtgtataagaggcag

SEQ ID NO: 19 - DNA, 19 bp, synthetic construct
  modified_base 17: mod_base = OTHER; note = 8-oxo-guanine
  source 1..19: mol_type = other DNA
agatgtgtataagagagag

SEQ ID NO: 20 - DNA, 19 bp, synthetic construct
  modified_base 18: mod_base = OTHER; note = 8-oxo-guanine
  source 1..19: mol_type = other DNA
agatgtgtataagagacgg

SEQ ID NO: 21 - DNA, 19 bp, synthetic construct
  modified_base 19: mod_base = OTHER; note = 8-oxo-guanine
  source 1..19: mol_type = other DNA
agatgtgtataagagacag

SEQ ID NO: 22 - DNA, 19 bp, synthetic construct
  modified_base 16: mod_base = i
  source 1..19: mol_type = other DNA
agatgtgtataagagncag

SEQ ID NO: 23 - DNA, 19 bp, synthetic construct
  modified_base 17: mod_base = i
  source 1..19: mol_type = other DNA
agatgtgtataagaganag

SEQ ID NO: 24 - DNA, 19 bp, synthetic construct
  modified_base 18: mod_base = i
  source 1..19: mol_type = other DNA
agatgtgtataagagacng